(19) United States
(12) Patent Application Publication
(10) Pub. No.: US 2023/0272460 A1
(43) Pub. Date: Aug. 31, 2023

(54) Method for Targeted Nucleic Acid Cleavage

(71) Applicant: Cambridge Enterprise Limited, Cambridge (GB)

(72) Inventors: Goncalo Bernardes, Cambridge (GB); Sigitas Mikutis, Cambridge (GB); Konstantinos Tzelepis, Cambridge (GB); Madoka Hazemi, Cambridge (GB)

(21) Appl. No.: 18/020,484

(22) PCT Filed: Aug. 12, 2021
(86) PCT No.: PCT/EP2021/072517

§ 371 Date: Feb. 9, 2023

(30) Foreign Application Priority Data
Aug. 12, 2020 (EP) 20386040.8

(51) Int. Cl. C12Q 1/6841; C12Q 1/6823; C12Q 1/6886

(52) U.S. Cl. CPC C12Q 1/6841; C12Q 1/6823; C12Q 1/6886; C12Q 1/6851

(57) ABSTRACT

The present invention provides methods for the non-enzymatic cleavage of target nucleic acids, for example for use in epigenomic and epitranscriptomic mapping and therapy. The method comprises contacting a target nucleic acid molecule with a bifunctional probe comprising a cleavage group and a covalent binding group such that the bifunctional probe covalently binds to the target nucleic acid molecule and cleaves the 5 target nucleic acid molecule bound thereto. Also provided is a method of selectively cleaving a target nucleic acid in a cell, a method for determining the modification of nucleic acid molecules by a nucleic acid modification enzyme in a cell, and a bifunctional probe for use in the methods.

### FIELD

The present invention relates to methods for the non-enzymatic cleavage of target nucleic acids, for example for use in epigenomic and epitranscriptomic mapping and therapy.

### BACKGROUND

Advances in nucleic acid manipulation and editing technologies have revolutionised the way biological research is conducted. RNA interference and shRNA expression systems have proven invaluable for target validation as well as elucidation of the role particular genes play in molecular diseases (Zamore, Phillip D., et al. Cell 101.1 (2000): 25-33). More recently, CRISPR-based technologies have enhanced the ability to manipulate DNA (Gasiunas, Giedrius, et al. PNAS USA 109.39 (2012): E2579-E2586; Jinek, Martin, et al. Science 337.6096 (2012): 816-821) and RNA (Cox, David BT, et al. Science 358.6366 (2017): 1019-1027) even further, empowering simpler systems such as gene knock-out cells and enabling large and elaborate CRISPR-Cas9-based genetic screening approaches (Tzelepis, Konstantinos, et al. Cell Reports 17.4 (2016): 1193-1205). However, the most commonly used nucleic acid manipulation technologies are genetic and it is challenging if not outright impossible to apply them to more complex biological systems such as rare populations, animal models and whole tissues, and yet even harder to develop therapeutics based on these technologies (Bobbin, Maggie L. et al Annual Review of Pharmacology and Toxicology 56 (2016): 103-122). Therefore, there is a pressing need to develop new small molecule-based technologies, which would enable manipulation of nucleic acids in yet unexplored contexts.

Conversely, the human cell has evolved a number of mechanisms to manipulate nucleic acids, including mechanisms which involve small molecules-cofactors. For example, RNA methylases METTL3 (Bokar, J. A., et al. RNA 3.11 (1997): 1233-1247) and METTL16 (Pendleton, Kathryn E., et al. Cell 169.5 (2017): 824-835) utilise cofactor SAM to deposit methyl groups on the N6 position on select adenosines across many different species of RNA. The resulting N6-methyladenosine mark regulates many aspects of the RNA lifecycle and activity, with sets of writer, eraser and reader enzymes to translate this functionality into biological function. Interest in this functionality has led to development of several m⁶A mapping methods, with m6A-seq (Dominissini, Dan, et al. Nature 485.7397 (2012): 201-206), MeRIP-seq (Meyer, Kate D., et al. Cell 149.7 (2012): 1635-1646.) and miCLIP (Linder, Bastian, et al. Nature methods 12.8 (2015): 767-772) being the most common. All of these mapping methods are antibody-based and hence suffer from several intrinsic caveats; high-input requirements, antibody binding biases as well as batch-to-batch variations and the inability to map the enzymatic activity were the key limiters of these methods. Furthermore, these techniques rely on m6A antibodies and hence they cannot be translated for use with other RNA modifications if an antibody is not available.

### SUMMARY

The present inventors have discovered that nucleic acids can be cleaved non-enzymatically using a bifunctional probe. This may be useful in a range of applications, including epigenetic and epitranscriptomic mapping and therapy.

Aspects of the invention provide a method for cleaving a target nucleic acid molecule comprising:
contacting the target nucleic acid molecule with a bifunctional probe having the formula:

C-L-B
where C is a cleavage moiety, L is a linker and B is a binding moiety; such that the bifunctional probe covalently binds to the target nucleic acid molecule, and;
allowing the bifunctional probe to cleave the target nucleic acid molecule covalently bound thereto.

Preferably, the cleavage moiety is substituted or unsubstituted imidazole.

Preferably, the target nucleic acid molecule is contacted with the bifunctional probe within a cell.

The bifunctional probe covalently binds to the target nucleic acid molecule. For example, the target nucleic acid molecule may be tagged with a partner moiety (P) to facilitate covalent binding of the bifunctional probe.

For example, a target nucleic acid molecule tagged with a partner moiety may be contacted with a bifunctional probe having the formula (1):

C-L-B_{C} (1)
where C is a cleavage moiety, L is a linker and B_{C} is a binding moiety comprising a reactive group that reacts with the partner moiety to covalently bind the bifunctional probe to the target nucleic acid molecule. The bifunctional probe may be allowed to cleave the nucleic acid molecule covalently bound thereto.

In preferred embodiments, the cleavage moiety is substituted or unsubstituted imidazole. Accordingly, the bifunctional probe has the formula (2):

I-L-B_{C} (2)
where I is a substituted or unsubstituted imidazole, L is a linker and B_{C} is a binding moiety comprising a reactive group that reacts with the partner moiety; such that the bifunctional probe covalently binds to the target nucleic acid molecule.

The target nucleic acid molecule may be tagged with the partner moiety by a nucleic acid modification enzyme. A method may comprise contacting the target nucleic acid molecule with a nucleic acid modification enzyme and a cofactor analogue comprising the partner moiety, such that the target nucleic acid is tagged with the partner moiety by the nucleic acid modification enzyme.

In some embodiments, the cofactor analogue may be introduced into a cell that comprises the target nucleic acid molecule and the nucleic acid modification enzyme.

In other embodiments, the cofactor analogue may be generated intracellularly from a cofactor analogue precursor. For example, a method may comprise introducing the cofactor analogue precursor into a cell comprising the target nucleic acid molecule and the nucleic acid modification enzyme, such that the cofactor analogue precursor is converted into the cofactor analogue within the cell.

In some preferred embodiments, the target nucleic acid may be an RNA molecule and the nucleic acid modification enzyme may be an RNA methyltransferase, preferably an S-adenosyl-L-methionine (SAM) dependent RNA methyltransferase. For example, a method for cleaving a target RNA molecule in a cell may comprise;
introducing a methionine analogue comprising a partner moiety into the cell,
   such that the methionine analogue is adenosylated in the cell to generate an S-adenosylmethionine analogue and;
   the S-adenosylmethionine analogue acts in combination with an RNA methyltransferase to tag the target RNA molecule with the partner moiety,
introducing into the cell a bifunctional probe having the formula (1):

C-L-B_{C} (1)
where C is a cleavage moiety, L is a linker and B_{C} is a binding moiety comprising a reactive group that reacts with the partner moiety; such that the bifunctional probe covalently binds to the target nucleic acid molecule, and;
allowing the bifunctional probe to cleave the RNA molecule bound thereto.

In some preferred embodiments, the methionine analogue is PropSeMet, the S-adenosylmethionine analogue is SeAdoYn, the partner moiety is a propargyl tag and the bifunctional probe has the formula (3):

C-L-N₃ (3)
where C is a cleavage moiety and L is a linker.

In some preferred embodiments, the bifunctional probe has the formula (5):

I-[PEG]ₙ—N₃ (5)
where I is a substituted or unsubstituted imidazole group, PEG is polyethylene glycol unit and n is from 2 to 10.

In some preferred embodiments, the target nucleic acid may be an RNA molecule and the nucleic acid modification enzyme may be an RNA acetyltransferase, preferably an acetyl-coenzyme A dependent RNA acetyltransferase. For example, a method for cleaving a target RNA molecule in a cell may comprise;
introducing an acetate analogue comprising a partner moiety into the cell,
   such that the acetate analogue is thioesterified with coenzyme A in the cell to generate an acetyl-CoA analogue; and,
   the acetyl-CoA analogue acts in combination with an RNA acetyltransferase to tag the target RNA molecule with the partner moiety,
introducing into the cell a bifunctional probe having the formula (1):

C-L-B_{C} (1)
where C is a cleavage moiety, L is a linker and B_{C} is a binding moiety comprising a reactive group that reacts with the partner moiety; such that the bifunctional probe covalently binds to the target nucleic acid molecule, and;
allowing the bifunctional probe to cleave the RNA molecule bound thereto.

In some preferred embodiments, the acetate analogue is a C₁₋₆ alkyl 3-butynoate ester, such as ethyl-3-butynoate, the acetyl-coenzyme A analogue is 3-butynoyl-coenzyme A, the partner moiety is a an alkynyl tag and the bifunctional probe has the formula (3):

C-L-N₃ (3)
where C is a cleavage moiety and L is a linker.

In some preferred embodiments, the acetate analogue is a C₁₋₆ alkyl 4-pentynoate ester, such as ethyl-4-pentynoate, the acetyl-coenzyme A analogue is 4-pentynoyl-coenzyme A, the partner moiety is an alkynyl tag and the bifunctional probe has the formula (3):

C-L-N₃ (3)
where C is a cleavage moiety and L is a linker.

In some preferred embodiments, the bifunctional probe has the formula (5):

I—[PEG]ₙ—N₃ (5)
where I is a substituted or unsubstituted imidazole group, PEG is polyethylene glycol unit and n is from 2 to 10.

In some preferred embodiments, the target nucleic acid may be an RNA molecule and the nucleic acid modification enzyme may an RNA glycosyltransferase. For example, a method for cleaving a target RNA molecule in a cell may comprise;
introducing an acetylated monosaccharide analogue comprising a partner moiety into the cell,
   such that the acetylated monosaccharide analogue is deacetylated in the cell to generate a monosaccharide analogue, and optionally the monosaccharide analogue is incorporated in a glycan; and,
   the monosaccharide analogue, or the glycan comprising the monosaccharide analogue, acts in combination with an RNA glycosyltransferase to tag the target RNA molecule with the partner moiety,
introducing into the cell a bifunctional probe having the formula (1):

C-L-B_{C} (1)
where C is a cleavage moiety, L is a linker and B_{C} is a binding moiety comprising a reactive group that reacts with the partner moiety; such that the bifunctional probe covalently binds to the target nucleic acid molecule, and;
allowing the bifunctional probe to cleave the RNA molecule bound thereto.

In some preferred embodiments, the acetylated monosaccharide analogue is an acylated mannose analogue, such as N-azidoacetylmannosamine-tetraacetylated (Ac₄ManNAz), the monosaccharide analogue is a neuraminic acid analogue, such as N-azidoacetylneuraminic acid (Neu₅Az), or a glycan comprising the neuraminic acid analogue, the partner moiety is an azide tag, and the bifunctional probe has the formula (3A):

C-L-B_{C≡C} (3A)
where C is a cleavage moiety and L is a linker, and B_{C≡C} is a binding moiety comprising an alkyne group that reacts with the partner moiety; such that the bifunctional probe covalently binds to the target nucleic acid molecule.

In some preferred embodiments, the acetylated monosaccharide analogue is an acetylated glucose analogue, such as 1,3,4,6-tetra-O-acetyl-azidoacetylglucosamine (Ac₄GlcNAz), the monosaccharide analogue is a glucose analogue, such as azidoacetylglucosamine (GlcNAz), or a glycan comprising the glucose analogue, the partner moiety is an azide tag, and the bifunctional probe has the formula (3A):

C-L-B_{C≡C} (3A)
where C is a cleavage moiety and L is a linker, and B_{C≡C} is a binding moiety comprising an alkyne group that reacts with the partner moiety; such that the bifunctional probe covalently binds to the target nucleic acid molecule.

In some preferred embodiments, the acetylated monosaccharide analogue is an acetylated fucose analogue, such as 6-azidofucose-tetraactylated (Ac₄FucAz), the monosaccharide analogue is a fucose analogue, such as 6-azidofucose (FucAz), or a glycan comprising the fucose analogue, the partner moiety is an azide tag, and the bifunctional probe has the formula (3A):

C-L-B_{C≡C} (3A)
where C is a cleavage moiety and L is a linker, and B_{C≡C} is a binding moiety comprising an alkyne group that reacts with the partner moiety; such that the bifunctional probe covalently binds to the target nucleic acid molecule.

In some preferred embodiments, the bifunctional probe has the formula (3B):

C-L-DBCO
where C is a cleavage moiety, L is a linker, and DBCO is a dibenzocyclooctyne group or a DBCO derivatives such as DBCO-amine or DBCO-carbamate.

In some preferred embodiments, the bifunctional probe has the formula (5A):

I—[PEG]ₙ-DBCO (5A)
where I is a substituted or unsubstituted imidazole group, PEG is polyethylene glycol unit and n is from 2 to 10, and DBCO is a dibenzocyclooctyne group or a DBCO derivatives such as DBCO-amine or DBCO-carbamate.

Other aspects and embodiments of the invention are described in more detail below.

### BRIEF DESCRIPTION OF THE FIGURES

FIG. 1 shows a schematic of a methylated RNA editing platform (referred to as Surrogate-Click-Degradation-Sequencing or Slick-Seq). (a) The proposed mechanism of action of Slick-Seq. (b) Conversion of PropSeMet into SeAdoYn and subsequent introduction of propargyl groups into RNA. (c) Functionalization of propargylated RNA with the click-degrader. (d) Proposed mechanism of the general base RNA degradation pathway. (e) Copper-mediated RNA degradation pathway.

FIG. 2 shows a study of the chemical mechanism of click-degraders. (a) Time-dependent degradation of Click-degrader 1 functionalized RNA 11-mer using at 37° C., n=2. (b) Extent of RNA degradation in 14 h at 37° C., pH 7.5, n=2. (c) Extent of RNA degradation in 14 h at 37° C., pH 3.0, n=2. (d) Extent of RNA degradation at neutral and acidic conditions, n=2. (e) Extent of RNA degradation in 14 h at 37° C., pH 7.5 using PEG linkers of differing lengths. Click-degraders 1, 2 and 3 have linkers with 6, 4 and 2 PEG subunits, respectively, n=2. Error bars represent SD.

FIG. 3 shows the elucidation of the relationship between m6A writers and methylation in mRNAs and IncRNAs using Slick-Seq. (a) Slick-Seq workflow. (b) Western blots demonstrate the extent of METTL3 and METTL16 depletion in conditional knock-down MOLM-13 cells treated with PropSeMet. Application of click degrader does not significantly alter the levels of these MTases. (c) Heatmap showing decrease of methylated mRNA levels upon clicking and rescue of METTL3-dependent transcripts upon METTL3 depletion. (d) Overlap of m6A-containing mRNAs determined via m6A miCLIP and METTL3 mRNA substrates determined via Slick-Seq. (e) RT-qPCR-based Slick-Seq validation of a panel of genes, n=3. (f) RT-qPCR-based Slick-Seq validation of a panel of genes in METTL3 depleted cells, n=3. (g) Genome browser snapshot of NEAT1. Applying click machinery diminishes the WT but not METTL3 or METTL16 levels. p values determined with one-tailed t-test. ns=not significant. Error bars represent SE.

FIG. 4 shows widespread m⁶A mark in introns and intergenic regions revealed by Slick-Seq. (a) Intronic peaks in the first intron of FLI1. (b) Intronic peaks in the first intron of CADM1 in three isogenic cell models. Intronic peaks are abolished specifically in METTL16-KD cells. (c) Overlap between METTL3- and METTL16-dependent intronic peaks. (d) Distribution of METTL3-dependent peaks in intronic and intergenic regions. (e) Distribution of METTL16-dependent peaks in intronic and intergenic regions. (f) Validation of dependence of intronic peaks on RNA methylases METTL3 and METTL16, n=3. (g) RT-qPCR-based validation of a panel intronic peaks, n=3. (h) Results of m⁶A-RIP in cells with methylated introns removed via dual gRNA system, n=3. (i) Results of m⁶A-RIP in cells with depleted METTL3, n=3. (j) Results of m⁶A-RIP in cells with depleted METTL16. RASA3 peak 2 is not affected by the knock-down illustrating that the observed effect is enzyme-specific, n=3. ns=not significant (p≥0.05). Error bars represent SD.

FIG. 5 shows LCMS traces supporting the functionalisation of RNA with click-degrader 1 and degradation of functionalised RNA on incubation. (a) Chromatogram of the propargylated RNA oligo (top), after functionalisation with click-degrader 1 (middle) and 14 h incubation at 37° C. (bottom). (b) Chromatogram of the non-propargylated RNA oligo (top), after functionalisation with click-degrader 1 (middle) and 14 h incubation at 37° C. (bottom). (c) Chromatogram of the propargylated RNA oligo (top), after functionalisation with click-degrader 1 via treatment with milder CuAAC conditions (100 μM CuSO4, 300 μM THPTA, 400 μM click-degrader, 5 mM NaAsc, 10 min), equivalent to cellular CuAAC conditions. (d) Logarithmic (first order) fit of time-dependent RNA degradation. n=2. (e) Calibration curve of click-degrader 1-functionalised RNA oligomer (iii). (f) Calibration curve of non-functionalised RNA (i). (g) Calibration curve of click-degrader 2-functionalised RNA oligomer (iv). (h) Calibration curve of click-degrader 3-functionalised RNA oligomer (v). Error bars represent SD.

FIG. 6 shows the relationship between m6A writers and methylation in mRNAs and IncRNAs using Slick-Seq. (a) Heatmap showing downregulation of methylated mRNAs and rescue of METTL16-dependent transcripts upon METTL16 depletion. (b) Overlap of m6A-containing mRNAs determined via miCLIP and METTL16 mRNA substrates determined via Slick-Seq. (c) RT-qPCR-based Slick-Seq validation of a panel of genes in METTL16 deficient cells, n=3. (d) Overlap between METTL3 and METTL16 mRNA substrates determined via Slick-Seq. (e) Overlap of m6A-containing IncRNAs determined via miCLIP and METTL3 IncRNA substrates determined via Slick-Seq. (f) Overlap of m6A-containing IncRNAs determined via miCLIP and METTL16 IncRNA substrates determined via Slick-Seq. (g) Overlap between METTL3 and METTL16 IncRNA substrates determined via Slick-Seq. (h) RT-qPCR-based Slick-Seq validation of a panel of IncRNAs in three isogenic cell lines, column order (left to right): Scramble CTRL, Scramble Click, shMETT3 CTRL, shMETTL3 Click, shMETTL16 CTRL, shMETTL16 Click (right), n=3. Error bars represent SD.

FIG. 7 shows examples of intronic peak snapshots. (a) Intronic peaks in FL/1, METTL16 dependent. (b) Intronic peaks in RASA3, METTL16 dependent. (c) Intronic peaks in DCP1B, METTL3 and METTL16 dependent. (d) Intronic peaks in an intergenic region in chromosome 4, METTL16 dependent.

FIG. 8 shows motif analysis for variations of the DRACH and TACAG motifs. (a) Consensus sequences found in METTL3-dependent peaks. (b) Consensus sequence found in METTL16-dependent peaks. (c) Overlap between m6A sites determined via miCLIP and METTL3-dependent intronic peaks determined via Slick-Seq, by considering miCLIP peaks exact, extended to both directions by 2000 and 5000 base pairs. Vertical lines at right indicate experimentally determined overlaps, curves at left indicate distributions of 100 simulations of randomly generated m6A sites. (d) Overlap of m6A sites determined via miCLIP and METTL16-dependent intronic peaks determined via Slick-Seq, similar to (c). (e) Distribution of Slick-Seq-determined METT3-dependent peaks around m6A sites found via miCLIP. (f) Distribution of Slick-Seq-determined METT16-dependent peaks around m6A sites found via miCLIP.

FIG. 9 shows the connection between METTL16 and intronic polyadenylation sites. (a) Overlap of intron polyadenylation (IPA) sites determined via 3′-seq in primary CLL cell and METTL16-dependent intronic peaks determined via Slick-Seq. Red lines indicate experimentally determined overlaps, black curves indicate distributions of 100 simulations of randomly generated IPA sites. (b) Overlap of IPA sites determined via 3′-seq in primary CLL cells and METTL3-dependent intronic peaks determined via Slick-Seq. (c) Distribution of distances between IPA sites and METTL16- or METTL3-dependent intronic peaks.

FIG. 10 provides a schematic overview of the chemical synthesis of PropSeMet (2), click-degraders (7, 8, and 9) and orthogonally-protected N⁶-propargyladenosine (16).

FIG. 11 shows (a) Chromatogram illustrates specific RNA degradation of propargylated RNA when the degradation is carried out on a mixture of propargylated and non-propargylated RNA oligo. (b) Quantification of bottom chromatogram on FIG. 11A. (c) The effect of 1 mM Cu(II), Fe(II) and Zn(II) on the activity of click-degrader 1 over 14 hours.

FIG. 12 provides a schematic overview of the chemical synthesis of ethyl-3-butynoate (17), click-degrader (7), click-degrader (20) and Ac₄FucAz (21).

FIG. 13 shows a schematic diagram of an acetylated RNA editing platform (referred to as acetylated Click-Sequencing or acCLICK-seq). (a) Proposed mechanism of enzymatic labelling of acetylated RNA with ethyl-3-butynoate followed by click-degradation action. (b) Conversion of ethyl-3-butynoate into 3-butynoyl-CoA for subsequent hijacking of RNA acetyltransferase to introduce alkyne group onto RNA. Copper mediated click reaction (CuAAC) by azide bearing click-degrader would then promote immediate RNA degradation. (c) Proposed mechanisms of RNA degradation via general base 2-0′ proton subtraction approach (top) and copper-mediated Cu(I) complex formation approach (bottom).

FIG. 14 shows a validation study on modified RNA transcripts performed using acCLICK-seq. (a) The workflow of acCLICK-Seq in cells. (b) RT-qPCR-based acCLICK-Seq validation of 18S and BRD4 genes in WT cells; Relative expression in treated ‘click’ cells is compared to the control (ethyl-3-butynoate probed and non-clicked) cells, n=3; *=p≤0.05, *** =p≤0.001, ns=not significant (p≥0.05). Error bars represent SD. (c) RT-qPCR-based acCLICK-Seq validation of 18S and BRD4 genes in NAT10 knocked-out cells; Relative expression in treated ‘click’ cells is compared to the control (ethyl-3-butynoate probed and non-clicked, n=3, Error bars represent SD.

FIG. 15 shows a schematic diagram of an glycosylated RNA editing platform (referred to as glycosylated Click-Sequencing or glycoCLICK-seq). (a) Proposed mechanism of enzymatic labelling of glycoRNA with azide-labelled sugar analog followed by click-degradation action. (b) Conversion of Ac₄ManAz into Neu₅Az (N-Acetylneuraminic acid azide) and subsequent introduction into glycan then attachment to RNA. Copper-free click reaction (SPAAC) by DBCO-based click degrader would then promote immediate base-promoted RNA degradation.

FIG. 16 shows (a) schematic diagram of a reaction between ManNAz or ManNAc with a click degrader to form a click product, and (b) HPLC chromatogram for DBCO-based click-degrader only (top) and after click functionalization with ManNAz under DMEM cell media +4% DMSO at 37° C. in 30 min (bottom). (c) HPLC chromatogram for DBCO-based click-degrader only (top) and after click functionalization with control ManNAc under DMEM cell media +4% DMSO at 37° C. in 30 min (bottom).

FIG. 17 shows a validation study on reported glycoRNA transcripts performed using glycoCLICK-seq. (a) GlycoCLICK-Seq workflow (Top). Selection of the sugar analogues for probing and click-degrader for facilitating GlycoCLICK-Seq (Bottom). (b) RT-qPCR-based validation; Relative expression in treated ‘click’ cells is compared to the control non-treated cells across a panel glycoRNA transcripts from GlycoCLICK-Seq platform using Ac₄ManNAz (top left), Ac₄FucAz (top right), Ac₄GIcNAz (bottom middle) n=3, *=p≤0.05, ** =p≤0.01, *** =p≤0.001, ns=not significant (p>0.05). Error bars represent SD.

### DETAILED DESCRIPTION

This invention relates to the finding that a bifunctional probe can be used as a catalytic agent to non-enzymatically cleave a target nucleic acid molecule. The selective cleavage of target nucleic acid molecules using a bifunctional probe as described herein may be useful in epigenetic and epitranscriptomic analysis bifunctional mapping, as well as therapy for example anti-viral therapy.

The invention provides a method for cleaving a target nucleic acid molecule. The method comprises contacting the target nucleic acid molecule with a bifunctional probe such that the bifunctional probe covalently binds to the target nucleic acid molecule, and allowing the bifunctional probe to cleave the target nucleic acid molecule bound thereto.

Binding of the bifunctional probe to the target nucleic acid may proceed via an intermediate species. That is, a target nucleic acid molecule may be cleaved as described herein by a method that comprises binding the target nucleic acid molecule to a bifunctional probe to produce an intermediate having the formula (6):

C-L-B_{A}˜NA (6)
where C is a cleavage moiety, L is a linker, B_{A} is a binding moiety that is bound to the target nucleic acid, ˜ is a covalent bond and NA is the target nucleic acid; and
allowing the bifunctional probe to cleave the target nucleic acid molecule.

In some embodiments, the target nucleic acid molecule may be contacted with the bifunctional probe in solution.

More preferably, the target nucleic acid molecule may be contacted with the bifunctional probe within a cell (i.e. intracellularly). The cell may be in vitro and may be an isolated cell, for example an isolated cell line or cell isolated from an individual (from a tissue sample, such as a biopsy).

Suitable cells may include mammalian, preferably human cells. Cells may include somatic and germ-line cells and may be at any stage of development, including fully or partially differentiated cells or non-differentiated or pluripotent cells, including stem cells, such as adult or somatic stem cells, foetal stem cells or embryonic stem cells. For example, cells may include neural cells, including neurons and glial cells, contractile muscle cells, smooth muscle cells, liver cells, hormone synthesising cells, sebaceous cells, pancreatic islet cells, adrenal cortex cells, fibroblasts, keratinocytes, endothelial and urothelial cells, osteocytes, and chondrocytes. In some embodiments, cells may be associated with a disease condition, for example cancer cells, such as carcinoma, sarcoma, lymphoma, blastoma or germ-line tumour cells, and cells with the genotype of a genetic disorder, such as Huntington's disease, cystic fibrosis, sickle cell disease, phenylketonuria, Down syndrome or Marfan syndrome.

The target nucleic acid molecule may be an endogenous nucleic acid that is present in the cell. The bifunctional probe may be an exogenous molecule. A method may comprise introducing the bifunctional probe into the cell and allowing it to covalently bind to the target nucleic acid molecule.

The target nucleic acid molecule may be a DNA or RNA molecule. Suitable target RNA molecules may include mRNA and long non-coding RNA (IncRNA). The RNA molecule may comprise intronic and intergenic regions.

The bifunctional probe has the formula:

C-L-B
where C is a cleavage moiety, L is a linker and B is a binding moiety.

The cleavage moiety of the bifunctional probe comprises a reactive group capable of reacting with a target nucleic acid molecule to cleave the target nucleic acid molecule. Typically, the cleavage moiety is capable of abstracting a proton from the hydroxyl group at the 2′ position of a ribose sugar. Optionally, the cleavage moiety is capable of binding copper to induce copper-mediated RNA degradation (Li, Zhong-Rui, et al. Nat Chem 11.10 (2019): 880-889; Wong, K, et al. Can J Biochem 52.11 (1974): 950-958; Subramaniam, Siddharth, et al. F1000Research 4 (2015)).

The cleavage moiety may comprise a basic group. That is, the cleavage moiety may comprise a group capable of accepting a hydrogen cation (H⁺). Typically, the basic group may be capable of donating an electron pair.

The basicity of a group may be quantitatively assessed using the pKa of the associated conjugate acid. That is, the basicity of basic group [B] may be assessed using the pKa of the conjugate acid [BH]⁺. The pKa of the conjugate acid may be known or it may be determined using standard techniques, such as acid-base titration. The cleavage moiety may comprise a basic group having a conjugate acid with a pKa of 6.0 or greater, for example 6.5 or greater or 7.0 or greater. Without wishing to be bound by theory, the inventors believe the basic residues having a pKa value above this threshold are capable of deprotonating the hydroxyl group at the 2′ position of a ribose sugar in order to permit cleavage of the phosphodiester backbone within a target nucleic acid.

Preferably, the cleavage moiety may comprise a nitrogen atom having a lone electron pair. Cleavage groups comprise a nitrogen atom having a lone electron pair are typically capable of coordinating copper.

The cleavage moiety may be or comprise a heteroaryl group comprising a nitrogen atom having a lone electron pair. A heteroaryl group is an aryl group comprising an aromatic ring in which one or more ring atoms are heteroatoms, for example N, O and S. The heteroaryl group may be a C₅₋₁₅ heteroaryl group. In this context, the prefix (e.g. C₁₋₁₅) denotes the number or range of ring atoms, whether carbon atoms or heteroatoms. The heteroaryl group may be monocyclic, or it may comprise two or more rings.

Examples of monocyclic C₅₋₁₅ heteroaryl groups comprising a suitable nitrogen atom include those derived from imidazole (1,3-diazole), triazole, tetrazole, pyridine (azine), and pyrazine (1,4-diazine).

The heteroaryl group may be part of a fused ring system. In a fused ring system the heteroaryl group comprises two or more rings, wherein at least one of the rings is an aromatic ring in which one or more ring atoms are heteroatoms, and wherein each ring shares two adjacent ring atoms with each neighbouring (fused) ring. Thus, the bridgehead atoms are directly bonded.

Examples of C₅₋₁₅ heteroaryl groups comprising a suitable nitrogen atom and a fused ring include those derived from (C₉) indole, indoline, isoindoline, purine, benzimidazole, azaindole, benzotriazole; (C₁₀) quinoline, isoquinoline, quinoxaline, phthalazine, quinazoline, naphthyridine, pyridopyrimidine, pyridopyrazine, pteridine; (C₁₁) benzodiazepine; (C₁₃) perimidine, pyridoindole; and (C₁₄) phenanthroline.

The heteroaryl group may be unsubstituted, or it may be substituted one, two or three C₁₋₆ alkyl groups, which may be the same or different.

An alkyl group is a monovalent saturated hydrocarbon group. The alkyl group may be a C₁₋₆ alkyl group, for example C₁₋₄alkyl group. In this context, the prefix (e.g. C₁₋₆) denotes the number of carbon atoms in the hydrocarbon backbone. The alkyl group may be linear or branched.

Examples of C₁₋₆ linear alkyl groups include methyl (-Me), ethyl (-Et), n-propyl (-nPr), n-butyl (-nBu), n-pentyl (-Amyl) and n-hexyl. Examples of C₁₋₆ branched alkyl groups include iso-propyl (-iPr), iso-butyl (-iBu), sec-butyl (-sBu), tert-butyl (-tBu), iso-pentyl, neo-pentyl, iso-hexyl and neo-hexyl.

Preferably, the cleavage moiety is selected from substituted or unsubstituted imidazole (1,3-diazole), triazole, benzimidazole and azaindole.

More preferably, the cleavage moiety is selected from substituted or unsubstituted imidazole (1,3-diazole). In such cases, the bifunctional probe may be referred to as an imidazole probe. Such an imidazole probe may be represented by the formula:

I-L-B
where I is a substituted or unsubstituted imidazole group, L is a linker and B is a binding moiety.

Imidazole has a pKa close to 7. Imidazole, in the form of histidine, is the active group in many ribonucleases. Imidazole is known to chelate copper. Some nucleic acid-cleaving natural products, such as Bleomycin A5, have an imidazole group for transition metal binding.

In some preferred embodiments, the cleavage moiety is selected from the groups represented by formula (1) to (III):

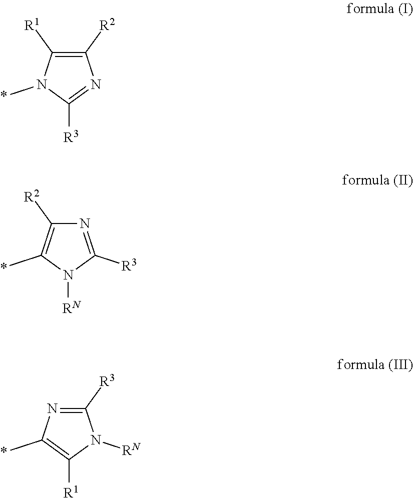

where: R¹, R² and R³ each independently represent a hydrogen atom or a C₁₋₆ alkyl group,
R^{N} represents a hydrogen atom or a C₁₋₆ alkyl group, and
* represents the attachment position with the remainder of the probe (typically the linker unit L).

In such cases, the bifunctional probe may be referred to as an imidazole probe.

Preferably, the cleavage moiety is a group represented by formula (1).

More preferably, R¹, R², R³ and R^{N} each independently represent a hydrogen atom or a C₁₋₄ alkyl group.

Even more preferably, R¹, R², R³ and R^{N} each independently represent hydrogen. In such case, the cleavage moiety is an unsubstituted imidazole group. That is, the cleavage moiety is represented by formula (IV):

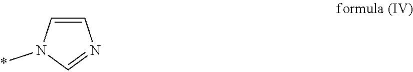

where * represents the attachment position with the remainder of the probe (typically the linker unit L).

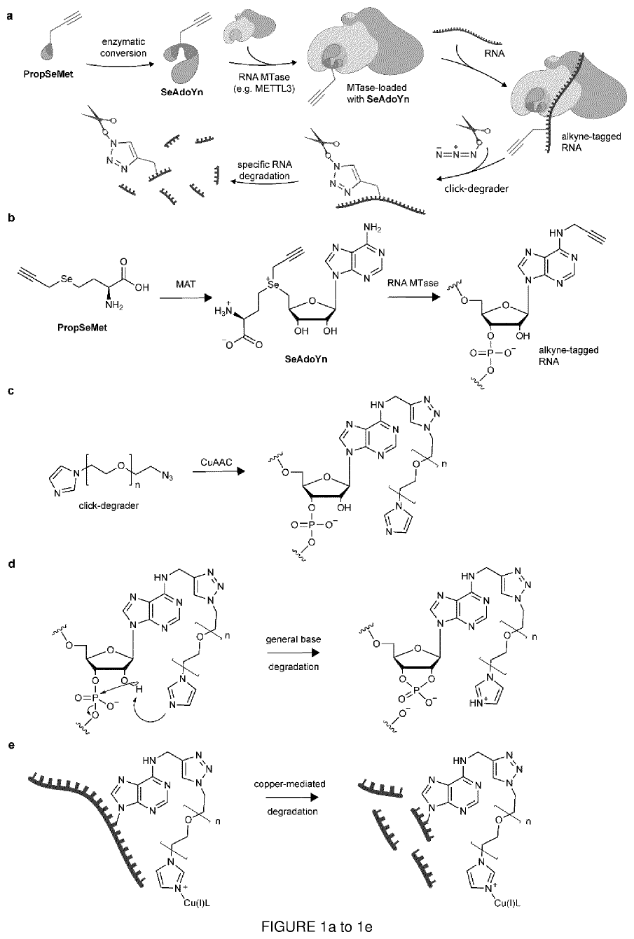

When covalently bound to the target nucleic acid molecule through the linker and binding moiety, the imidazole group reacts with the target nucleic acid molecule to cleave one or more phosphodiester bonds, thereby causing degradation of the target nucleic acid molecule. For example, the imidazole group of the bound probe may abstract a proton from the 2′O position on the nucleic acid molecule leading to cleavage of a phosphodiester bond in the target nucleic acid molecule. A possible mechanism is shown in FIG. 1d. In addition, the imidazole group may form a copper complex which cleaves a phosphodiester bond in the target nucleic acid molecule. A possible mechanism is shown in FIG. 1e.

The linker L of the bifunctional probe comprises a group for connection (i.e. covalent connection) of the cleavage moiety (C) to the binding moiety (B). Suitable linkers are well known in the art.

Typically, the linker comprises a divalent group in which one of the free valencies forms part of a single bond to the cleavage moiety (C) and the remaining free valency forms part of a single bond to the binding moiety (B).

Preferably, the linker is a stable linker. That is, the linker comprises a group that is not substantially cleaved or degraded in vivo. A stable linker is typically unreactive at physiological pH, and not substantially degraded by enzymatic action in vivo.

Typically, the linker is a flexible linker. That is, the linker permits the cleavage moiety (C) and binding moiety (B) to move relative to each other with a large degree of freedom.

Typical linkers comprise groups selected from alkylene, heteroalkylene, cycloalkylene, heterocycloalkylene, arylene and heteroarylene. Mixed linkers comprising different groups in covalent connection, such as alkylene-arylene (aralkylene) and heteroalkylene-arylene, may be permitted.

An alkylene (alkanediyl) group is a divalent saturated hydrocarbon group in which the two free valencies each form part of a single bond to an adjacent atom. The alkylene group may be a C₁₋₆ alkylene group, for example, a C₁₋₄, C₁₋₃ or a C₁₋₂ alkylene group. In this context, the prefix (e.g. C₁₋₆) denotes the number of atoms in the hydrocarbon backbone. The alkylene group may be linear or branched. Examples of linear alkylene groups include methanediyl (methylene bridge), ethane-1,2-diyl (ethylene bridge), propane-1,3-diyl, butan-1,4-diyl, pentan-1,5-diyl and hexan-1,6-diyl. Examples of branched alkylene groups include ethane-1,1-diyl and propane-1,2-diyl.

A heteroalkylene group is an alkylene group in which one or more carbon atoms is replaced with a heteroatom, for example N, O and S. The heteroalkylene group may be a C₁₋₆ heteroalkylene group, for example, a C₁₋₄, C₁₋₃ or a C₁₋₂ heteroalkylene group. In this context, the prefix (e.g. C₁₋₆) denotes the number of atoms in the heteroalkylene backbone, whether carbon atoms or heteroatoms. The heteroalkylene group may be linear or branched. Examples of linear heteroalkylene groups include those derived from oxymethylene (e.g. polyoxymethylene, POM), ethylene glycol (e.g. polyethylene glycol, PEG), ethylenimine (e.g. linear polyethylenimine, PEI; polyaziridine) and tetramethylene glycol (e.g. polytetramethylene glycol, PTMEG; polytetrahydrofuran). Examples of branched heteroalkylene groups include those derived from propylene glycol (e.g. polypropylene glycol PPG). Where a nitrogen atom is present in a heteroalkylene group, that nitrogen atom may be unsubstituted (NH) or optionally substituted with an alkyl group. Where a sulfur atom is present in a heteroalkyl group, that sulfur atom may be S, S(O) or S(O)₂.

A cycloalkylene group is a divalent saturated hydrocarbon group which comprises a ring in which all of the ring atoms are carbon atoms, and in which the two free valencies each form part of a single bond to an adjacent atom. The cycloalkylene group may be a C₅₋₆ cycloalkylene group. In this context, the prefix (e.g. C₅₋₆) denotes the number or range of ring atoms. The cycloalkylene group may be monocyclic. Examples of monocylic cycloalkylene groups include 1,3-cyclopentylene and 1,4-cyclohexylene.

A heterocycloalkylene (heterocyclene) group is a cycloalkylene group in which one or more carbon atoms is replaced with a heteroatom, for example N, O and S, or in which one or more carbon atoms has an oxo substituent (═O). The heterocycloalkylene group may be a C₅₋₆ heterocycloalkylene group. In this context, the prefix (e.g. C₅₋₆) denotes the number or range of ring atoms, whether carbon atoms or heteroatoms. The heterocycloalkylene group may be monocyclic. Where a nitrogen atom is present in a heteroalkylene group, that nitrogen atom may be unsubstituted (NH) or optionally substituted with an alkyl group. Where a sulfur atom is present in a heteroalkyl group, that sulfur atom may be S, S(O) or S(O)₂.

An arylene group is a divalent hydrocarbon group comprises an aromatic ring in which all of the ring atoms are carbon atoms, and in which the two free valencies each form part of a single bond to an adjacent atom. The arylene group may be a C₆₋₁₀ arylene group. In this context, the prefix (e.g. C₆₋₁₀) denotes the number or range of ring atoms. The arylene group may be monocyclic, or it may comprise two or more rings. Examples of monocyclic arylene groups include 1,4-phenylene. Examples of bicyclic arylene groups include 2,6-naphthylene.

A heteroarylene group is an arylene group comprising an aromatic ring in which one or more ring atoms are heteroatoms, for example N, O and S, or in which one or more carbon atoms has an oxo substituent (═O). The heteroarylene group may be a C₆₋₁₀ heteroarylene group. In this context, the prefix (e.g. C₆₋₁₀) denotes the number or range of ring atoms, whether carbon or heteroatom. The heteroarylene group may be monocyclic, or it may comprise two or more rings. Examples of monocyclic heteroarylene groups include pyrrolylene and pyridylene.

Preferred linkers comprise groups selected from alkylene and heteroalkylene. More preferred linkers comprise heteroalkylene groups. Even more preferred linkers comprise polyalkylene glycol groups. Most preferred linkers comprise polyethylene glycol (PEG) or polypropylene glycol (PPG) groups.

The linker may vary in length. Typically, the linker contains two or more repeated units. Typically, the linker contains at most ten repeat units. That is, the linker may be represented as:

—Lₙ—
where n is 2 to 10.

Preferably, n is between 2 and 8. More preferably, n is between 4 and 8. Even more preferably n is 6.

Particularly preferred linkers (L) are derived from polyethylene glycol groups [PEG], which comprise ethylene oxide units (—CH₂CH₂O—). Attachment of a PEG linker to the binding group (or cleavage group) typically displaces the terminal oxygen atom, to leave a terminal ethylene group (—CH₂CH₂—).

A particularly preferred linker is derived from a polyethylene glycol comprising six ethylene oxide units [PEG]₆. A linker derived from [PEG]₆ has the formula —(CH₂CH₂O)₅—CH₂CH₂—.

The binding moiety of the bifunctional probe comprises a group capable of covalently binding to the target nucleic acid molecule.

The bifunctional probe covalently binds to the target nucleic acid. For example, a method for cleaving a target nucleic acid molecule as described herein may comprise reacting the target nucleic acid molecule with a bifunctional probe to produce an intermediate having formula (6):

C-L-B_{A}-NA (6)
where C is a cleavage moiety, L is a linker, B_{A} is a binding moiety that is covalently bound to the target nucleic acid and NA is the target nucleic acid; and,
allowing the bifunctional probe to cleave the target nucleic acid molecule.

In some preferred embodiments, the target nucleic acid may be tagged with a partner moiety P. The partner moiety of the target nucleic acid may react with the binding moiety of the bifunctional probe to covalently bind the bifunctional probe to the target nucleic acid molecule. For example, a method may comprise contacting a target nucleic acid molecule tagged with a partner moiety with a bifunctional probe having the formula (1):

C-L-B_{C} (1)
where C is a cleavage moiety, L is a linker and B_{C} is a binding moiety comprising a reactive group that reacts with the partner moiety to covalently bind the bifunctional probe to the target nucleic acid molecule; and,
allowing the bifunctional probe to cleave the target nucleic acid molecule covalently bound thereto.

Typically, the binding moiety B_{C} reacts with the partner moiety P to form the group BA which covalently binds the probe to the target nucleic acid. The covalent binding moiety (B_{C}) may comprise any reactive group that is capable of forming a covalent bond with a partner moiety.

Covalent linkage of the binding moiety to the partner moiety may be achieved through any convenient chemical coupling procedure. Preferably, a bioorthogonal chemical reaction is used. That is, a chemical reaction that can occur inside a living system (e.g. a cell) without interfering with native biochemical processes within the system. Preferably, click chemistry is used. In such cases, the binding moiety and the partner moiety represent any two groups capable of reacting in a click reaction.

The covalent binding moiety may be or comprise a group selected from azido (—N₃), nitrone (R′C═N⁺R″O—, where R″ is not H), nitrile oxide (—C≡N⁺—O⁻) or tetrazine.

Preferably, the covalent binding moiety is or comprises an azido group (—N₃).

The partner moiety may comprise any reactive group that is capable of forming a covalent bond with a binding moiety.

The partner moiety may be or comprise a group selected from alkynyl, alkenyl or isocyanide (-N+=C—).

Alternatively, the binding moiety and partner moieties may be reversed. Here, the covalent binding moiety may be or comprise a group selected from alkynyl, alkenyl or isocyanide (—N⁺≡C⁻); and the partner moiety may be or comprise a group selected from azido (—N₃), nitrone (R′C═N⁺R″O⁻, where R″ is not H), nitrile oxide (—C≡N⁺—O⁻) or tetrazine.

In some embodiments, the covalent binding moiety is or comprises an alkyne, and the partner moiety is or comprises an azido (—N₃) group.

In some embodiments, the covalent binding moiety is or comprises a nitrone (—R′C═N⁺R″O⁻, where R″ is not H), and the partner moiety is or comprises an alkynyl group.

In some embodiments, the covalent binding moiety is or comprises a nitrone (—R′C═N⁺R″O⁻, where R″ is not H), and the partner moiety is or comprises an alkenyl group.

In some embodiments, the covalent binding moiety is or comprises tetrazine and the partner moiety is or comprises an alkynyl group.

In some embodiments, the covalent binding moiety is or comprises tetrazine and the partner moiety is or comprises an isocyanide (—N⁺≡C⁻) group.

In some embodiments, the covalent binding moiety is or comprises a nitrile oxide (—C≡N⁺—O⁻) and the partner moiety is or comprises an alkenyl group.

In some embodiments, the partner moiety is or comprises an alkyne, and the covalent binding moiety is or comprises an azido (—N₃) group.

In some embodiments, the partner moiety is or comprises a nitrone (—R′C═N⁺R″O⁻, where R″ is not H), and the covalent binding moiety is or comprises an alkynyl group.

In some embodiments, the partner moiety is or comprises a nitrone (—R′C═N⁺R″O⁻, where R″ is not H), and the covalent binding moiety is or comprises an alkenyl group.

In some embodiments, the partner moiety is or comprises tetrazine and the covalent binding moiety is or comprises an alkynyl group.

In some embodiments, the partner moiety is or comprises tetrazine and the covalent binding moiety is or comprises an isocyanide (—N⁺≡C⁻) group.

In some embodiments, the partner moiety is or comprises a nitrile oxide (—C≡N⁺—O⁻) and the covalent binding moiety is or comprises an alkenyl group.

An alkynyl (alkyne) group is a monovalent unsaturated hydrocarbon group containing one or more carbon-carbon triple bonds. The alkenyl group may be a C₂₋₂₀ alkenyl group, for example a C₂₋₁₀, C₂₋₆ or a C₂₋₄ alkenyl group. The alkenyl group may be linear or branched. The alkenyl group may be incorporated into a ring system. Incorporation into a ring system permits the use of a copper-free, strain-promoted click reaction.

Examples of linear alkynyl groups include ethynyl and 2-propynyl (propargyl). Examples of alkynyl groups incorporated into a ring system include cyclooctyne (OCT), aryl-less octyne (ALO), monofluorinated cyclooctyne (MOFO), difluorocyclooctyne (DIFO), dimethoxyazacyclooctyne (DIMAC), dibenzocyclooctyne (DIBO), dibenzoazacyclooctyne (DIBAC), biarylazacyclooctynone (BARAC), bicyclononyne (BCN), 2,3,6,7-tetramethoxydibenzocyclooctyne (TMDIBO), sulfonylated dibenzocyclooctyne (S-DIBO), carboxymethylmonobenzocyclooctyne (COMBO) and pyrrolocyclooctyne (PYRROC).

Examples of alkynyl groups incorporated into ring systems include strained alkynyl group, such as a dibenzocyclooctynyl (DBCO) group, biarylazacyclooctynonyl (BARAC) group, or difluorocyclooctyne (DIFO) group. Derivatives of these groups may be used. An example of a suitable derivative is a dibenzocyclooctyne-amino group (DBCO-amine) or dibenzocyclooctyne-carbamate group (DBCO-carbamate):

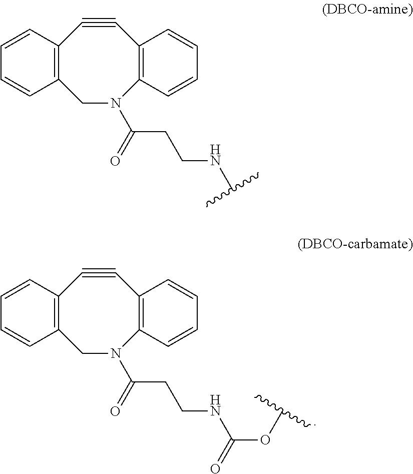 [SMILES: *NCCC(=O)N1Cc2ccccc2C#Cc2ccccc21 ; *OC(=O)NCCC(=O)N1Cc2ccccc2C#Cc2ccccc21]

An alkenyl group is a monovalent unsaturated hydrocarbon group containing one or more carbon-carbon double bonds. The alkenyl group may be a C₂₋₂₀ alkenyl group, for example a C₂₋₁₀, C₂₋₆ or a C₂₋₄ alkenyl group. The alkenyl group may be incorporated into a ring system. Incorporation into a ring system permits the use of a copper-free, strain-promoted click reaction.

Examples of alkenyl groups incorporated into a ring system include norbornene, oxanorborandiene and trans-cycloctene.

Preferably, the partner moiety is or comprises an alkynyl group. More preferably, the partner moiety is or comprises a propargyl group.

Where the covalent binding moiety comprises an azido group (—N₃), the binding moiety may react with a partner moiety comprising an alkyne (C≡C) through an azide-alkyne cycloaddition (AAC), for example a copper (I)-catalyzed azide-alkyne cycloaddition (CuAAC) or a strain-promoted azide-alkyne cycloaddition (SPAAC). In such cases, the product of the reaction between the binding moiety and the partner moiety is a 1,2,3-triazole moiety. That is, reaction may proceed through the intermediate:

C-L-B_{A}-NA
where C is a cleavage moiety, L is a linker, BA is a 1,2,3-triazole moiety and NA is the target nucleic acid.

Where the covalent binding moiety comprises a nitrone group (—R′C═N⁺R″O⁻, where R″ is not H, such as where R′ is H or C₁₋₄ alkyl and R″ is C₁₋₄ alkyl), the binding moiety may react with a partner moiety comprising an alkyne through a 1,3-dipolar cycloaddition. In such cases, the product of the reaction between the binding moiety and the partner moiety is an isoxazoline moiety. That is, reaction may proceed through the intermediate:

C-L-B_{A}-NA
where C is a cleavage moiety, L is a linker, B_{A} is a isoxazoline moiety and NA is the target nucleic acid.

Where the covalent binding moiety is a nitrile oxide group (—C≡N⁺—O⁻), the binding moiety may react with a partner moiety comprising an alkene (C═C) such as norbornene through a 1,3-dipolar cycloaddition. In such cases, the product of the reaction between the binding moiety and the partner moiety is an isoxazole moiety.

That is, reaction may proceed through the intermediate:

C-L-B_{A}-NA
where C is a cleavage moiety, L is a linker, B_{A} is an isoxazole moiety and NA is the target nucleic acid.

Where the covalent binding moiety is a tetrazine group, the binding moiety may react with a partner moiety comprising an alkene (C═C) such as trans-cyclooctene through an inverse-electron demand Diels Alder reaction followed by a retro-Diels Alder reaction. In such cases, the product of the reaction between the binding moiety and the partner moiety is a dihydropyridazine moiety. That is, reaction may proceed through the intermediate:

C-L-B_{A}-NA
where C is a cleavage moiety, L is a linker, BA is a dihydropyridazine moiety and NA is the target nucleic acid.

Alternatively, a tetrazine covalent binding moiety may reaction with a partner moiety comprising an isocyanide moiety (—N⁺≡C—) through a [4+1] cycloaddition followed by a retro-Diels Alder reaction. In such cases, the product of the reaction between the binding moiety and the partner moiety is a pyrazole moiety. That is, reaction may proceed through the intermediate:

C-L-B_{A}-NA
where C is a cleavage moiety, L is a linker, BA is a pyrazole moiety and NA is the target nucleic acid.

Where the covalent binding moiety comprises an alkynyl group, the binding moiety may react with a partner moiety comprising an azido group (—N₃) through an azide-alkyne cycloaddition (AAC), for example a copper (I)-catalyzed azide-alkyne cycloaddition (CuAAC) or a strain-promoted azide-alkyne cycloaddition (SPAAC). Preferably, when the binding moiety comprises an alkynyl group, the binding moiety reacts with the partner moiety through a strain-promoted azide-alkyne cycloaddition (SPAAC). In such cases, the product of the reaction between the binding moiety and the partner moiety is a 1,2,3-triazole moiety. That is, the reaction may proceed through the intermediate:

C-L-B_{A}-NA
where C is a cleavage moiety, L is a linker, BA is a 1,2,3-triazole moiety and NA is the target nucleic acid.

In a preferred embodiment, the partner moiety is an alkynyl group, such as a propargyl group, and the covalent binding moiety is an azido group. In such cases, the method may comprise reacting a target nucleic acid molecule tagged with an alkynyl group with a bifunctional probe having the formula (3):

C-L-N₃ (3)
where C is a cleavage moiety and L is a linker, such that the azido group reacts with the alkynyl group to covalently bind the bifunctional probe to the target nucleic acid molecule; and,
allowing the bifunctional probe to cleave the target nucleic acid molecule covalently bound thereto.

In a further preferred embodiment, the method may comprise reacting a target nucleic acid molecule tagged with an alkynyl group with a bifunctional probe having the formula (5):

I—[PEG]ₙ—N₃ (5)
where I is a substituted or unsubstituted imidazole group, PEG is polyethylene glycol unit and n is from 2 to 10, such that the azido group reacts with the alkynyl group to covalently bind the bifunctional probe to the target nucleic acid molecule; and,
allowing the bifunctional probe to cleave the target nucleic acid molecule covalently bound thereto.

In other preferred embodiment, the partner moiety may be or comprise an azido (—N₃) group, and the covalent binding moiety is or comprises an alkynyl group (—C≡C—). In such cases, the method may comprise reacting a target nucleic acid molecule tagged with an azido group with a bifunctional probe having the formula (3A):

C-L-B_{C≡C} (3A)
where C is a cleavage moiety and L is a linker, and B_{C≡C} is a binding moiety comprising an alkyne group, such that alkyne group reacts with the azido group to covalently bind the bifunctional probe to the target nucleic acid molecule; and,
allowing the bifunctional probe to cleave the RNA molecule bound thereto.

In another preferred embodiment, the method may comprise reacting a target nucleic acid molecule tagged with an azido group with a bifunctional probe having the formula (3B):

C-L-DBCO (3B)
where C is a cleavage moiety, L is a linker, and DBCO is a dibenzocyclooctyne group, or a dibenzocyclooctyne derivatives such as DBCO-amine or DBCO-carbamate, such that the dibenzocyclooctyne group or derivative reacts with the azido group to covalently bind the bifunctional probe to the target nucleic acid molecule; and,
allowing the bifunctional probe to cleave the RNA molecule bound thereto.

In another preferred embodiment, the method may comprise reacting a target nucleic acid molecule tagged with an azido group with a bifunctional probe having the formula 5A):

I—[PEG]ₙ-DBCO (5A)
where I is a substituted or unsubstituted imidazole group, PEG is polyethylene glycol unit and n is from 2 to 10, and DBCO is a dibenzocyclooctyne group, or a dibenzocyclooctyne derivatives such as DBCO-amine or DBCO-carbamate, such that the dibenzocyclooctyne group or derivative reacts with the azido group to covalently bind the bifunctional probe to the target nucleic acid molecule; and,
allowing the bifunctional probe to cleave the RNA molecule bound thereto.

Optionally, the method may comprise reacting the target nucleic acid with the bifunctional probe and copper, such as a copper (I) salt. Suitable copper (I) salts may be use directly. Examples of copper (I) salts that may be used directly include cuprous bromide (CuBr) and cuprous iodide (Cul). Alternatively, suitable copper (1) salts may be generated in situ by reduction of copper (II) salts. Example copper (II) salts include copper sulfate (CuSO4) or copper acetate (Cu(OAc)₂). Example reducing agents include sodium ascorbate. Optimally, copper-binding ligands such as THPTA (Tris((1-hydroxy-propyl-1 H-1,2,3-triazol-4-yl)methyl) amine) may be used.

The use of copper may additionally benefit the cleavage reaction by promoting copper-mediated RNA degradation.

Optionally, the method may comprise reacting the target nucleic acid with the bifunctional probe and zinc, such as a zinc (II) salt. Examples of zinc (II) salts that may be used include zinc bromide (ZnBr₂), zinc chloride (ZnCl₂) and zinc iodide (Znl₂).

The use of zinc may benefit the cleavage reaction by promoting RNA degradation.

The target nucleic acid molecule may be tagged with the partner moiety by a nucleic acid modification enzyme. Suitable nucleic acid modification enzymes include RNA methyl transferases, such as METTL3 and METTL16; DNA methyl transferases, such as DNMT1, DNMT3a, DNMT3b, METTL4 and N6AMT1; DNA hydroxyl-methylation enzymes, such as TET1 and TET2; RNA acetyl transferases, such as N-acetyltransferase 10 (NAT10); and RNA glycosyltransferases, such as oligosaccharyltransferase (OST).

The target nucleic acid may comprise a site that is modified by the nucleic modification enzyme (i.e. a site of nucleic modification). For example, the site may be tagged by the nucleic modification enzyme with a chemical modifying group, such as a methyl, acetyl or glycosyl group. The nucleic acid modification enzyme may tag the target nucleic acid with the partner moiety at the site of nucleic modification. For example, an RNA methyl transferase may methylate the N6 position of an adenosine residue in a nucleic acid. In the methods described herein, the RNA methyl transferase may tag the target nucleic acid with the partner moiety at the N6 position of an adenosine residue in the target nucleic acid.

A method of selectively cleaving a target nucleic acid in a cell may comprise;
contacting in the cell a target nucleic acid molecule, a nucleic acid modification enzyme and a cofactor analogue comprising the partner moiety, such that the modification enzyme tags the target nucleic acid with the partner moiety;
introducing into the cell a bifunctional probe having the formula (1):

C-L-B_{C} (1)
where C is a cleavage moiety, L is a linker and B_{C} is a binding moiety comprising a reactive group that reacts with the partner moiety to covalently bind the bifunctional probe to the target nucleic acid molecule; and,
allowing the bifunctional probe to cleave the target nucleic acid molecule covalently bound thereto.

A cofactor is a non-protein compound that is required in order for the nucleic acid modification enzyme to be catalytically active. A cofactor may comprise a chemical modifying group, such as a methyl, acetyl or glycosyl group. The cofactor may donate the chemical modifying group, such that the nucleic acid modification enzyme attaches it to the target nucleic acid at the site of modification, for example to methylate, acetylate or glycosylate the target nucleic acid.

A cofactor analogue is a non-protein compound that acts as a cofactor for a nucleic acid modification enzyme i.e. the nucleic acid modification enzyme is catalytically active in the presence of the cofactor analogue. In some embodiments, the cofactor analogue may comprise a partner moiety. The co-factor analogue may donate the partner moiety, such that the nucleic acid modification enzyme attaches the partner moiety to the target nucleic acid at the site of modification.

Preferably, the target nucleic acid molecule is contacted with the nucleic acid modification enzyme and the cofactor analogue in a cell. The nucleic acid modification enzyme and the target nucleic acid molecule may be endogenous to the cell i.e. the nucleic acid modification enzyme and the target nucleic acid molecule may occur naturally in the cell.

In a preferred aspect of the invention, the target nucleic acid may be an RNA molecule and the nucleic acid modification enzyme may be an RNA methyltransferase. Suitable cofactor analogues for use with an RNA methyltransferase may include S-adenosylmethionine analogues, such as propargylic SeAdoYn (propargylic Se-adenosyl-L-selenomethionine) and propargylic SAdoYn (propargylic S-adenosyl-L-methionine):

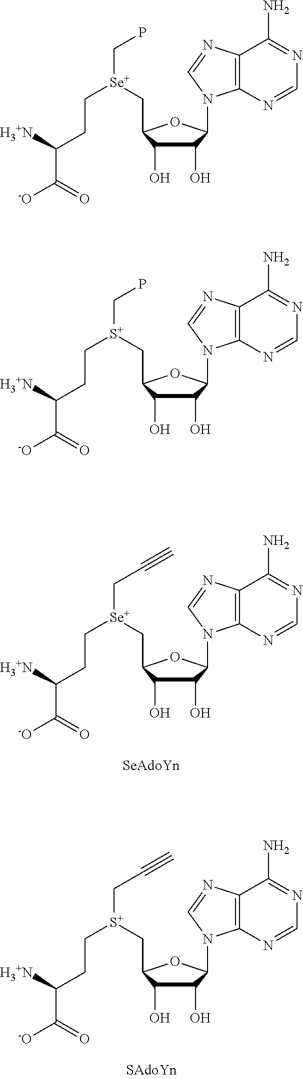

where P is a partner moiety comprising a reactive group that reacts with the binding moiety to covalently bind the bifunctional probe to the target nucleic acid molecule.

In some preferred embodiments, a method for selectively cleaving a target RNA molecule in a cell may comprise;
contacting in a cell the target RNA molecule, an RNA methyltransferase and an RNA methyltransferase co-factor analogue comprising a partner moiety, such that the RNA methyltransferase covalently tags the target RNA molecule with the partner moiety;
introducing into the cell a bifunctional probe having the formula (1):

C-L-B_{C} (1)
where C is a cleavage moiety, L is a linker and B_{C} is a binding moiety comprising a reactive group that reacts with the partner moiety to covalently bind the bifunctional probe to the target RNA molecule; and,
allowing the bifunctional probe to cleave the RNA molecule covalently bound thereto.

Suitable cofactor analogues may include S-adenosylmethionine (SAM) analogues, such as SeAdoYn. For example, a suitable method may comprise:
contacting in a cell the target RNA molecule, an RNA methyltransferase and a SAM analogue comprising an alkynyl group, for example SeAdoYn, such that the RNA methyltransferase covalently tags the target RNA molecule with the alkynyl group;
introducing into the cell a bifunctional probe having the formula (3):

C-L-N₃ (3)
where C is a cleavage moiety and L is a linker, such that the bifunctional probe reacts with the alkynyl group to covalently bind to the target RNA molecule, and;
allowing the bifunctional probe to cleave the RNA molecule.

In a preferred embodiment, the method may comprise:
contacting in a cell the target RNA molecule, an RNA methyltransferase and a SAM analogue comprising an alkynyl group, for example SeAdoYn, such that the RNA methyltransferase covalently tags the target RNA molecule with the alkynyl group;
introducing into the cell a bifunctional probe having the formula (5):

I—[PEG]ₙ—N₃ (5)
where I is a substituted or unsubstituted imidazole group, PEG is polyethylene glycol unit and n is from 2 to 10, such that the bifunctional probe reacts with the alkynyl group to covalently bind to the target RNA molecule, and;
allowing the bifunctional probe to cleave the RNA molecule.

In some embodiments, the cofactor analogue may be exogenous and may be introduced to the cell. For example, a method may comprise introducing the cofactor analogue to a cell.

In other embodiments, the cofactor analogue may be generated in the cell from an exogenous cofactor analogue precursor. For example, a method may comprise introducing a cofactor analogue precursor into the cell, such that the cofactor analogue precursor is converted in the cell into the cofactor analogue. Generating the cofactor analogue from an exogenous cofactor analogue precursor may be advantageous as the precursor may have a longer half-life (be a more stable compound).

A cofactor analogue precursor is a molecule that is metabolised by a cell to produce a cofactor analogue. For example, a cofactor analogue precursor, such as a methionine analogue, may be adenosylated in the cell by an endogenous adenosyltransferase, such as a methionine adenosyltransferase (MAT), to generate a cofactor analogue, such as an S-adenosylmethionine analogue.

In some aspects of the invention, the target nucleic acid may be an RNA molecule and the nucleic acid modification enzyme may be an RNA methyltransferase. Suitable cofactor analogues may include S-adenosylmethionine (SAM) analogues, such as SeAdoYn. Suitable cofactor analogue precursors for generating a SAM analogue in a cell may include methionine analogues, such as PropSeMet (propargylic-selenomethionine) and PropSMet (propargylic methionine):

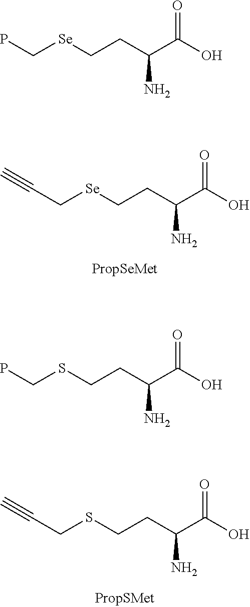

where P is a partner moiety comprising a reactive group that reacts with the binding moiety to covalently bind the bifunctional probe to the target nucleic acid molecule.

Accordingly, a method for selectively cleaving a target RNA molecule in a cell may comprise:
introducing a methionine analogue comprising a partner moiety into the cell,
   such that the cell adenosylates the methionine analogue to generate an S-adenosylmethionine analogue and;
   the S-adenosylmethionine analogue forms a cofactor for a RNA methyltransferase that tags the target RNA molecule with the partner moiety,
introducing into the cell a bifunctional probe having the formula (1):

C-L-B_{C} (1)
where C is a cleavage moiety, L is a linker and B_{C} is a binding moiety comprising a reactive group that reacts with the partner moiety to covalently bind the bifunctional probe to the target RNA molecule; and,
allowing the bifunctional probe to cleave the RNA molecule covalently bound thereto.

For example, a method for selectively cleaving a target RNA molecule in a cell may comprise:
introducing PropSeMet into the cell,
   such that the cell adenosylates the PropSeMet to generate SeAdoYn and;
   the SeAdoYn reacts with RNA methyltransferase to tag the 6 position of an adenosine in a target RNA molecule with a propargyl group,
introducing into the cell a bifunctional probe having the formula (3):

C-L-N₃ (3)
where C is a cleavage moiety and L is a linker; such that the bifunctional probe reacts with the propargyl tag to covalently bind to the target RNA molecule, and;
   allowing the bifunctional probe to cleave the RNA molecule covalently bound thereto.

In some preferred embodiments, the bifunctional probe has the formula (5):

I—[PEG]ₙ—N₃ (5)
where I is a substituted or unsubstituted imidazole group, PEG is polyethylene glycol unit and n is from 2 to 10.

In other aspects of the invention, the target nucleic acid may be a DNA molecule and the nucleic acid modification enzyme may be a DNA methyltransferase. The DNA methyltransferase may be a SAM-dependent DNA methyl transferase. Suitable cofactor analogues and cofactor analogue precursors for use with DNA methyltransferases are described above.

In other aspects of the invention, the target nucleic acid may be an RNA molecule and the nucleic acid modification enzyme may be an RNA acetyltransferase. Suitable cofactor analogues may include acetyl-CoA analogues, such as propargylic acetyl-CoA and 1-butynyl acetyl-CoA:

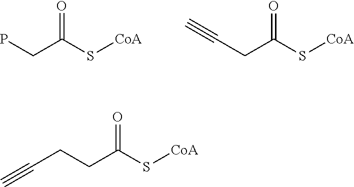

where P is a partner moiety comprising a reactive group that reacts with the binding moiety to covalently bind the bifunctional probe to the target nucleic acid molecule.

In these aspects, a method for selectively cleaving a target RNA molecule in a cell may comprise;
contacting in a cell the target RNA molecule, an RNA acetyltransferase and an RNA acetyltransferase co-factor analogue comprising a partner moiety, such that the RNA acetyltransferase covalently tags the target RNA molecule with the partner moiety;
introducing into the cell a bifunctional probe having the formula (1):

C-L-B_{C} (1)
where C is a cleavage moiety, L is a linker and B_{C} is a binding moiety comprising a reactive group that reacts with the partner moiety to covalently bind the bifunctional probe to the target RNA molecule; and,
allowing the bifunctional probe to cleave the RNA molecule covalently bound thereto.

Suitable RNA acetyltransferase cofactor analogues may include acetyl-coenzyme A (acetyl-CoA) analogues, such as 3-butynoyl-coenzyme A (3-butynoyl-CoA). For example, a suitable method may comprise:
contacting in a cell the target RNA molecule, an RNA acetyl transferase and an acetyl-CoA analogue comprising an alkynyl group, for example 3-butynoyl-CoA, such that the RNA acetyltransferase covalently tags the target RNA molecule with the alkynyl group;
introducing into the cell a bifunctional probe having the formula (3):

C-L-N₃ (3)
where C is a cleavage moiety and L is a linker, such that the bifunctional probe reacts with the alkynyl group to covalently bind to the target RNA molecule, and;
allowing the bifunctional probe to cleave the RNA molecule.

In a preferred embodiment, the method may comprise:
contacting in a cell the target RNA molecule, an RNA acetyltransferase and an acetyl-CoA analogue comprising an alkynyl group, for example 3-butynoyl-CoA, such that the RNA acetyltransferase covalently tags the target RNA molecule with the alkynyl group;
introducing into the cell a bifunctional probe having the formula (5):

I—[PEG]ₙ—N₃ (5)
where I is a substituted or unsubstituted imidazole group, PEG is polyethylene glycol unit and n is from 2 to 10, such that the bifunctional probe reacts with the alkynyl group to covalently bind to the target RNA molecule, and;
allowing the bifunctional probe to cleave the RNA molecule.

The cofactor analogue may be exogenous and may be introduced to the cell, or the cofactor analogue may be generated in the cell from an exogenous cofactor analogue precursor. The cofactor analogue precursor may be an acetate analogue, which may be thioesterified in the cell with an endogenous enzyme comprising a thiol group, such as coenzyme A, to generate a cofactor analogue, such as an acetyl-CoA analogue.

In some aspects of the invention, the target nucleic acid may be an RNA molecule and the nucleic acid modification enzyme may be an RNA acetyltransferase. Suitable cofactor analogue precursors for generating an acetyl-CoA analogue in a cell may include acetate analogues such as carboxylic and pyruvic acid and ester such as:

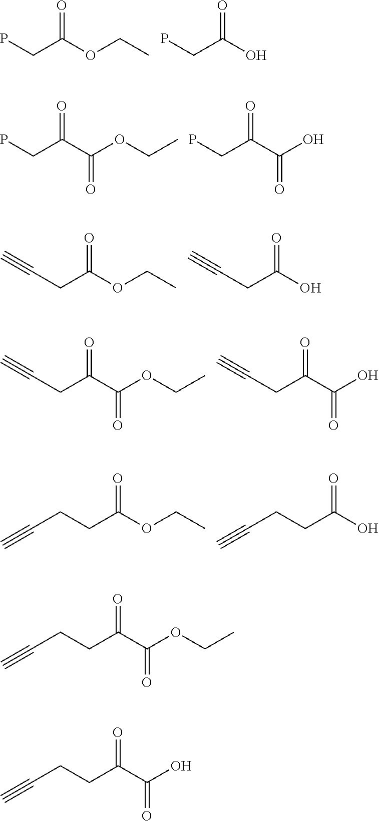

where P is a partner moiety comprising a reactive group that reacts with the binding moiety to covalently bind the bifunctional probe to the target nucleic acid molecule.

C₁₋₆ alkyl butynoate esters, C₁₋₆ alkyl pentynoate esters, pyruvic acid analogues and pyruvate ester analogues are preferred.

C₁₋₆ alkyl butynoate esters, such as ethyl-3-butynoate, and C₁₋₆ alkyl pentynoate esters, such as ethyl-4-pentynoate are particularly preferred.

Accordingly, a method for selectively cleaving a target RNA molecule in a cell may comprise:
introducing an acetate analogue comprising a partner moiety into the cell,
   such that the cell thioesterifies the acetate analogue to generate an acetyl-CoA analogue and;
   the acetyl-CoA analogue forms a cofactor for a RNA acetyltransferase that tags the target RNA molecule with the partner moiety,
introducing into the cell a bifunctional probe having the formula (1):

C-L-B_{C} (1)
where C is a cleavage moiety, L is a linker and B_{C} is a binding moiety comprising a reactive group that reacts with the partner moiety to covalently bind the bifunctional probe to the target RNA molecule; and,
allowing the bifunctional probe to cleave the RNA molecule covalently bound thereto.

For example, a method for selectively cleaving a target RNA molecule in a cell may comprise:
introducing ethyl-3-butynoate into the cell,
   such that the cell thioesterifies the ethyl-3-butynoate to generate 3-butynoyl-CoA and;
   the 3-butynoyl-CoA reacts with RNA acetyltransferase to tag the 4 position of a cytidine in a target RNA molecule with an alkynyl group, such as a 3-butynoyl group,
introducing into the cell a bifunctional probe having the formula (3):

C-L-N₃ (3)
where C is a cleavage moiety and L is a linker; such that the bifunctional probe reacts with the alkynyl tag to covalently bind to the target RNA molecule, and;
   allowing the bifunctional probe to cleave the RNA molecule covalently bound thereto.

In other embodiments, a method for selectively cleaving a target RNA molecule in a cell may comprise:
introducing ethyl-4-pentynoate into the cell,
   such that the cell thioesterifies the ethyl-4-pentynoate to generate 4-pentynoyl-CoA and;
   the 4-pentynoyl-CoA reacts with RNA acetyltransferase to tag the 4 position of a cytidine in a target RNA molecule with an alkynyl group, such as a 4-pentynoyl group,
introducing into the cell a bifunctional probe having the formula (3):

C-L-N₃ (3)
where C is a cleavage moiety and L is a linker; such that the bifunctional probe reacts with the alkynyl tag to covalently bind to the target RNA molecule, and;
allowing the bifunctional probe to cleave the RNA molecule covalently bound thereto.

In some preferred embodiments, the bifunctional probe has the formula (5):

I—[PEG]ₙ—N₃ (5)
where I is a substituted or unsubstituted imidazole group, PEG is polyethylene glycol unit and n is from 2 to 10.

In other aspects of the invention, the target nucleic acid may be a DNA molecule and the nucleic acid modification enzyme may be a DNA acetyltransferase. The DNA acetyltransferase may be an acetyl-CoA dependent DNA acetyltransferase. Suitable cofactor analogues and cofactor analogue precursors for use with DNA acetyltransferases are described above.

In other aspects of the invention, the target nucleic acid may be an RNA molecule and the nucleic acid modification enzyme may be an RNA glucosyltransferase. Suitable cofactor analogues for use with an RNA glycosyltransferase may include monosaccharide analogues, such as an analogue of N-acetylneuraminic acid (sialic acid), mannose, glucose or fucose comprising an azido (N₃) group, and glycans comprising a monomer unit derivable from these monosaccharide analogues. Examples of suitable monosaccharide analogues include N-azidoacetylneuraminic acid (Neu₅Az), N-azidoacetylmannosamine (ManNAz), N-azidoacetylglucosamine (GlcNAz), and 6-azidofucose (FucAz):

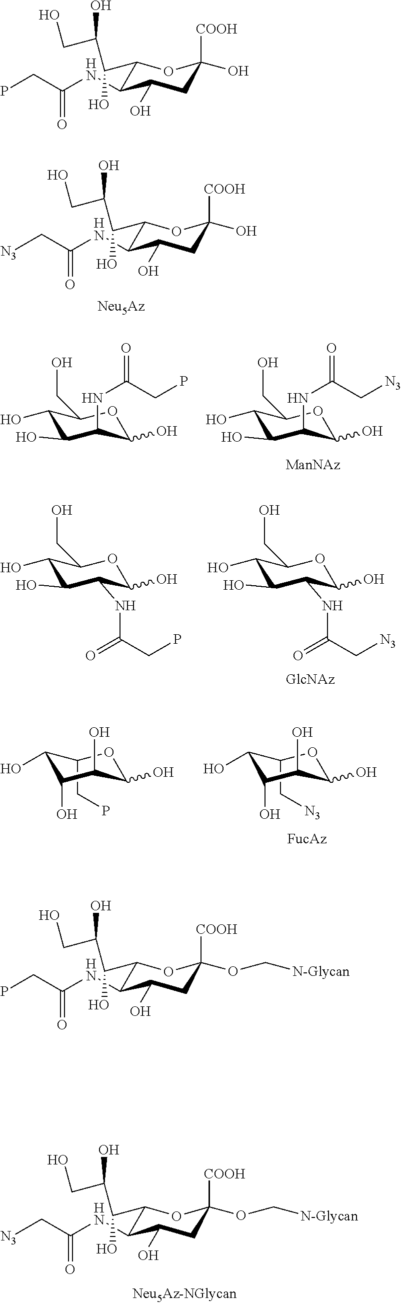

where P is a partner moiety comprising a reactive group that reacts with the binding moiety to covalently bind the bifunctional probe to the target nucleic acid molecule.

N-azidoacetylneuraminic acid (Neu₅Az), and glycans comprising Neu₅Az, are particularly preferred.

Accordingly, a method for selectively cleaving a target RNA molecule in a cell may comprise;
contacting in a cell the target RNA molecule, an RNA glycosyltransferase and an RNA glycosyltransferase co-factor analogue comprising a partner moiety, such that the RNA glycosyltransferase covalently tags the target RNA molecule with the partner moiety;
introducing into the cell a bifunctional probe having the formula (1):

C-L-B_{C} (1)
where C is a cleavage moiety, L is a linker and B_{C} is a binding moiety comprising a reactive group that reacts with the partner moiety to covalently bind the bifunctional probe to the target RNA molecule; and,
allowing the bifunctional probe to cleave the RNA molecule covalently bound thereto.

Suitable RNA glycosyltransferase cofactor analogues may include monosaccharide analogues, such as analogues of N-acetylneuraminic acid (sialic acid), mannose, glucose or fucose comprising an azido (N₃) group, and glycans comprising a monomer unit derivable from these monosaccharide analogues. For example, a suitable method may comprise:
contacting in a cell the target RNA molecule, an RNA glycosyltransferase and a monosaccharide analogue comprising an azido group, for example Neu₅Az, or a glycan comprising the monosaccharide analogue, such that the RNA glycosyltransferase covalently tags the target RNA molecule with the azido group;
introducing into the cell a bifunctional probe having the formula (3A):

C-L- C-L-B_{C≡C} (3A)
where C is a cleavage moiety and L is a linker, and B_{C≡C} is a binding moiety that comprises an alkynyl group, such that the bifunctional probe reacts with the azido group to covalently bind to the target RNA molecule, and;
allowing the bifunctional probe to cleave the RNA molecule.

In a preferred embodiment, the method may comprise:
contacting in a cell the target RNA molecule, an RNA glycosyltransferase and a monosaccharide analogue comprising an azido group, for example Neu₅Az, or a glycan comprising the monosaccharide analogue, such that the RNA glycosyltransferase covalently tags the target RNA molecule with the azido group;
introducing into the cell a bifunctional probe having the formula (3B):

C-L-DBCO (3B)
where C is a cleavage moiety, L is a linker, and DBCO is a dibenzocyclooctyne group or a DBCO derivatives such as DBCO-amine or DBCO-carbamate, such that the bifunctional probe reacts with the azido group to covalently bind to the target RNA molecule, and;
allowing the bifunctional probe to cleave the RNA molecule.

In a preferred embodiment, the method may comprise:
contacting in a cell the target RNA molecule, an RNA glycosyltransferase and a monosaccharide analogue comprising an azido group, for example Neu₅Az, or a glycan comprising the monosaccharide analogue, such that the RNA glycosyltransferase covalently tags the target RNA molecule with the azido group;
introducing into the cell a bifunctional probe having the formula (5A):

I—[PEG]ₙ-DBCO (5A)
where I is a substituted or unsubstituted imidazole group, PEG is polyethylene glycol unit and n is from 2 to 10, and DBCO is a dibenzocyclooctyne group or a DBCO derivatives such as DBCO-amine or DBCO-carbamate, such that the bifunctional probe reacts with the azido group to covalently bind to the target RNA molecule, and;
allowing the bifunctional probe to cleave the RNA molecule.

The cofactor analogue may be exogenous and may be introduced to the cell, or the cofactor analogue may be generated in the cell from an exogenous cofactor analogue precursor. The cofactor analogue precursor may be a monosaccharide analogue, which may be modified in the cell by an endogenous enzyme to generate the cofactor analogue.

Modification of the cofactor analogue precursor in the cell may comprise generating an N-acetylneuraminic acid analogue from the monosaccharide analogue, followed by incorporating the N-acetylneuraminic acid analogue in a glycan. For example, the cofactor precursor may be a mannose analogue, which may be metabolised to generate a N-acetylneuraminic acid analogue, and then incorporated in a glycan.

Modification of the cofactor analogue precursor in the cell may comprise modifying the monosaccharide analogue, followed by incorporating the metabolised monosaccharide analogue in a glycan. For example, the cofactor precursor may be an acetylated fucose analogue or an acetylated glucose analogue, which may be deacetylated in the cell to generate the monosaccharide analogue. The monosaccharide analogue may then be incorporated in a glycan.

In some aspects of the invention, the target nucleic acid may be an RNA molecule and the nucleic acid modification enzyme may be an RNA glycosyltransferase. Suitable cofactor analogues may include monosaccharide analogues, such as analogues of N-acetylneuraminic acid (sialic acid), mannose, glucose or fucose comprising an azido (N₃) group, and glycans comprising a monomer unit derivable from these monosaccharide analogues. Suitable cofactor analogue precursors for generating a monosaccharide analogue, or a glycan comprising the monosaccharide analogue, in a cell may include acetylated monosaccharide analogues, such as N-azidoacetylmannosamine-tetraacetylated (Ac₄ManNAz), N-azidoacetylglucosamine-tetraacetylated (Ac₄GlcNAz), or 6-azidofucose-tetraactylated (Ac₄FucAz):

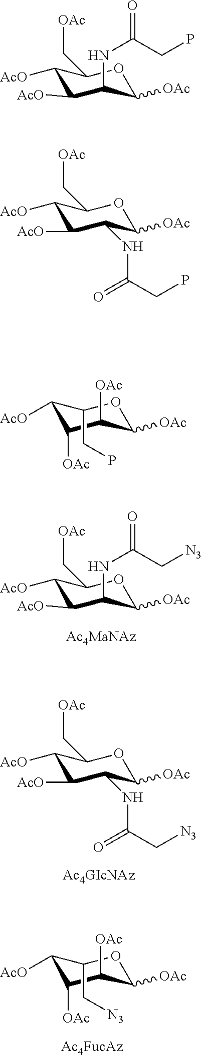

where P is a partner moiety comprising a reactive group that reacts with the binding moiety to covalently bind the bifunctional probe to the target nucleic acid molecule.

Accordingly, a method for selectively cleaving a target RNA molecule in a cell may comprise:
introducing an acetylated monosaccharide analogue comprising a partner moiety into the cell,
   such that the cell deacetylates the acetylated monosaccharide analogue to generate a monosaccharide analogue; and optionally incorporates the monosaccharide analogue in a glycan; and,
   the monosaccharide analogue or the glycan comprising the monosaccharide analogue forms a cofactor for a RNA glycosyltransferase that tags the target RNA molecule with the partner moiety, introducing into the cell a bifunctional probe having the formula (1):

C-L-B_{C} (1)
where C is a cleavage moiety, L is a linker and B_{C} is a binding moiety comprising a reactive group that reacts with the partner moiety to covalently bind the bifunctional probe to the target RNA molecule; and,
allowing the bifunctional probe to cleave the RNA molecule covalently bound thereto.

For example, a method for selectively cleaving a target RNA molecule in a cell may comprise:
introducing Ac₄ManNAz into the cell,
   such that the cell deacetylates the Ac₄ManNAz to form Neu₅Az; and optionally incorporates the Neu₅Az in a glycan; and,
   the Neu₅Az or glycan reacts with RNA glycosyltransferase to tag a nucleobase in a target RNA molecule with an azido group,
introducing into the cell a bifunctional probe having the formula (3A):

C-L-B_{C≡C} (3A)
where C is a cleavage moiety, L is a linker, and B_{C≡C} is a binding moiety comprising an alkynyl group; such that the bifunctional probe reacts with the azido tag to covalently bind to the target RNA molecule, and;
allowing the bifunctional probe to cleave the RNA molecule covalently bound thereto.

In some preferred embodiments, the bifunctional probe has the formula (3B):

C-L-DBCO (3B)
where C is a cleavage moiety, L is a linker, and DBCO is a dibenzocyclooctyne group or a DBCO derivatives such as DBCO-amine or DBCO-carbamate.

In some further preferred embodiments, the bifunctional probe has the formula (5A):

I—[PEG]ₙ-DBCO (5A)
where I is a substituted or unsubstituted imidazole group, PEG is polyethylene glycol unit, n is from 2 to 10 and DBCO is a dibenzocyclooctyne group or a DBCO derivatives such as DBCO-amine or DBCO-carbamate.

In other aspects of the invention, the target nucleic acid may be a DNA molecule and the nucleic acid modification enzyme may be a DNA glycosyltransferase. Suitable cofactor analogues and cofactor analogue precursors for use with DNA glycosyltransferase are described above.

Following selective cleavage of a target nucleic acid molecule by a bifunctional probe as described above, a method may comprise identifying the target nucleic acid molecule. This may be useful for example in the mapping of sites that are modified by a nucleic acid modification enzyme.

For example, a method may comprise determining the abundance or amount of one or more nucleic acid molecules in the cell. A reduction in the abundance or amount of a nucleic acid molecule in the cell relative to control cells may be indicative that the nucleic acid molecule is the target nucleic acid molecule that has been selectively cleaved by the bifunctional probe.

A method may comprise extracting the total nucleic acid, such as total DNA or total RNA, from a cell. The nucleic acid may be further analysed, for example to determine the abundance or amount of one or more nucleic acid molecules. For example, the extracted total nucleic acid may be sequenced and the sequence reads analysed.

Suitable methods of determining the abundance or amount of nucleic acid molecules in a cell are well known in the art and include RT-qPCR, RNA-sequencing (RNA-seq), next generation (NGS) and other sequencing techniques, such as Sanger sequencing, Tracking Indels by Composition (TIDE) (Brinkman et al Nucleic Acids Res. 2014 Dec. 16; 42(22): e168) and PCR analysis, In some embodiments, a method may comprise extracting nucleic acid molecules from the cell, sequencing the extracted nucleic acid molecules and determining the number of sequence reads (i.e. read count) for each extracted nucleic molecule to determine the abundance or amount of each nucleic acid molecule in the cell. In some embodiments, the raw read count may be normalised and expressed in RPKM (reads per kilobase of exon model per million reads) or FPKM (fragments per kilobase of exon model per million reads mapped). Suitable methods of sequencing and sequence analysis are well established in the art.

The abundance or amount of nucleic acid molecules labelled with a partner moiety using a co-factor analogue precursor, as described herein may be determined by any convenient technique, for example nanopore sensing (see for example Shi et al Anal Chem. 2017 Jan. 3; 89(1): 157-188.)

In some embodiments, the abundance or amount of a nucleic acid molecule in the cell may be determined relative to a control cell not subjected to selective cleavage as described above. For example, suitable control cells include (i) a cell that has been treated with a bifunctional probe but not a co-factor analogue or pre-cursor thereof (ii) a cell that has been treated with a co-factor analogue or pre-cursor thereof but not a bifunctional probe, as described above, and/or (iii) a cell that has not been treated with either a co-factor analogue or pre-cursor thereof or a bifunctional probe as described above. A reduction in the abundance or amount of a nucleic acid molecule in the cell relative to the control cells may be indicative that the nucleic acid molecule is modified by a modification enzyme in the cell.

In some embodiments, the abundance or amount of a nucleic acid molecule in the cell may be determined relative to a control cell in which a selected nucleic acid modification enzyme has been inactivated. A reduction in the abundance or amount of a nucleic acid molecule in the cell relative to the control cells may be indicative that the nucleic acid molecule is modified by the selected nucleic acid modification enzyme.

This may be useful for example in mapping the modification of nucleic acid in a cell by a nucleic acid modification enzyme.

A method for determining the modification of nucleic acid molecules by a nucleic acid modification enzyme in a cell may comprise
providing a first cell and a second cell, wherein the second cell has reduced or abolished expression or activity of the nucleic acid modification enzyme relative to the first cell,
introducing in the first and second cells a co-factor analogue precursor comprising a partner moiety,
   such that the co-factor analogue precursor is converted in the first and second cells into a co-factor analogue,
   said co-factor analogue being a co-factor for the nucleic acid modification enzyme, such that nucleic molecules in the cell that contain a site of modification are tagged with the partner moiety in the presence of the nucleic acid modification enzyme,
introducing into the cells a bifunctional probe having the formula (1):

C-L-B_{C} (1)
where C is a cleavage moiety, L is a linker and B_{C} is a binding moiety comprising a reactive group that reacts with the partner moiety to covalently bind the bifunctional probe to nucleic acid molecules tagged with the partner moiety,
allowing the bifunctional probe to cleave nucleic acid molecules in the first and seconds cells covalently bound to the bifunctional probe, and
identifying nucleic acid molecules which are present in a reduced amount in the first cell relative to the second cell,
wherein said identified one or more nucleic acid molecules contain a site of modification by the nucleic acid modification enzyme.

Nucleic acid modification enzymes may include RNA methyltransferases, such as SAM-dependent N6-adenosine methyltransferases, DNA methyltransferases, RNA glucosyltransferases, RNA acetyltransferases, RNA glycosyltransferases, DNA glycosyltransferases and DNA acetyltransferases.

For example, a method for determining the methylation of RNA molecules by an RNA methyltransferase in a cell may comprise;
providing a first cell and a second cell, wherein the second cell has reduced or abolished expression or activity of the RNA methyltransferase relative to the first cell,
introducing in the first and second cells a methionine analogue comprising a partner moiety,
   such that the methionine analogue is adenosylated in the first and second cells into a S-adenosylmethionine analogue,
   said S-adenosylmethionine analogue being a co-factor for the RNA methyltransferase, such that RNA molecules in the cell that contain a methylation site are tagged with the partner moiety in the presence of the RNA methyltransferase in the cell,
introducing into the cells a bifunctional probe having the formula (1):

C-L-B_{C} (1)
where C is a cleavage moiety, L is a linker and B_{C} is a binding moiety comprising a reactive group that reacts with the partner moiety to covalently bind the bifunctional probe to RNA molecules tagged with the partner moiety,
   allowing the bifunctional probe to cleave nucleic acid molecules in the first and second cells covalently bound to the bifunctional probe, and
   identifying RNA molecules which are present in a reduced amount in the first cell relative to the second cell,
   wherein said identified RNA molecules contain a site of methylation by the nucleic acid modification enzyme.

Preferred embodiments of the methionine analogue, S-adenosylmethionine analogue, and the bifunctional probe are set out above.

For example, a method for determining the acetylation of RNA molecules by an RNA acetyltransferase in a cell may comprise;
providing a first cell and a second cell, wherein the second cell has reduced or abolished expression or activity of the RNA acetyltransferase relative to the first cell,
introducing in the first and second cells an acetate analogue comprising a partner moiety,
   such that the acetate analogue is thioesterified in the first and second cells into an acetyl-CoA analogue,
   said acetyl-CoA analogue being a co-factor for the RNA acetyltransferase, such that RNA molecules in the cell that contain a acetylation site are tagged with the partner moiety in the presence of the RNA acetyltransferase in the cell,
introducing into the cells a bifunctional probe having the formula (1):

C-L-B_{C} (1)
where C is a cleavage moiety, L is a linker and B_{C} is a binding moiety comprising a reactive group that reacts with the partner moiety to covalently bind the bifunctional probe to RNA molecules tagged with the partner moiety,
   allowing the bifunctional probe to cleave nucleic acid molecules in the first and second cells covalently bound to the bifunctional probe, and
   identifying RNA molecules which are present in a reduced amount in the first cell relative to the second cell,
   wherein said identified RNA molecules contain a site of acetylation by the nucleic acid modification enzyme.

Preferred embodiments of the acetate analogue, acetyl-CoA analogue, and the bifunctional probe are set out above.

For example, a method for determining the glycosylation of RNA molecules by an RNA glycosyltransferase in a cell may comprise;
providing a first cell and a second cell, wherein the second cell has reduced or abolished expression or activity of the RNA glycosyltransferase relative to the first cell,
introducing in the first and second cells an acetylated monosaccharide analogue comprising a partner moiety,
   such that the acetylated monosaccharide analogue is deacetylated in the first and second cells into a monosaccharide analogue, and optionally the monosaccharide analogue is incorporated into a glycan,
   said monosaccharide analogue, or glycan comprising the monosaccharide analogue, being a co-factor for the RNA glycosyltransferase, such that RNA molecules in the cell that contain a glycosylation site are tagged with the partner moiety in the presence of the RNA glycosyltransferase in the cell,
introducing into the cells a bifunctional probe having the formula (1):

C-L-B_{C} (1)
where C is a cleavage moiety, L is a linker and B_{C} is a binding moiety comprising a reactive group that reacts with the partner moiety to covalently bind the bifunctional probe to RNA molecules tagged with the partner moiety,
   allowing the bifunctional probe to cleave nucleic acid molecules in the first and second cells covalently bound to the bifunctional probe, and
   identifying RNA molecules which are present in a reduced amount in the first cell relative to the second cell,
   wherein said identified RNA molecules contain a site of glycosylation by the nucleic acid modification enzyme.

Preferred embodiments of the monosaccharide analogue, acetylated monosaccharide analogue, and the bifunctional probe are set out above.

A further aspect of the present invention provides a bifunctional probe having the formula:

I-L-B
where I is a substituted or unsubstituted imidazole, L is a linker and B is a binding moiety that covalently binds to a nucleic acid molecule.

In a preferred embodiment, the bifunctional probe has the formula (4):

I-L-N₃ (4)
where I is a substituted or unsubstituted imidazole, L is a linker and N₃ is an azido group.

In some preferred embodiments, the bifunctional probe has the formula (5):

I—[PEG]ₙ—N3 (5)
where I is a substituted or unsubstituted imidazole group, PEG is polyethylene glycol unit and n is from 2 to 10.

In another preferred embodiment, the bifunctional probe has the formula (4A):

I-L-DBCO (4A)
where I is a substituted or unsubstituted imidazole, L is a linker and DBCO is a dibenzocyclooctyne group or a DBCO derivatives such as DBCO-amine or DBCO-carbamate.

In some preferred embodiments, the bifunctional probe has the formula (5A):

I—[PEG]ₙ-DBCO (5A)
where I is a substituted or unsubstituted imidazole group, PEG is polyethylene glycol unit, n is from 2 to 10 and DBCO is a dibenzocyclooctyne group or a DBCO derivatives such as DBCO-amine or DBCO-carbamate.

In another preferred embodiment, the bifunctional probe is selected from compounds Deg-1 to Deg-3 set out below:

| Compound | Structure |
|---|---|
| Deg-1 | 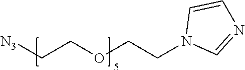 |
| Deg-2 | 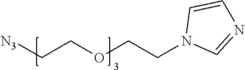 |
| Deg-3 | 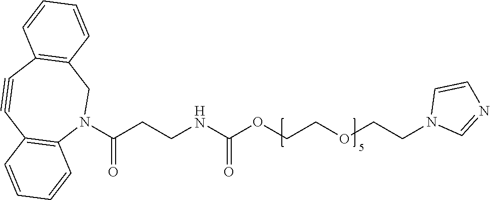 |

RNA molecules may be identified by sequencing RNA molecules in the first and second cells and determining the number of sequences reads of the RNA molecules in the first and second cells. For example, total RNA in the cells may be sequence. Suitable techniques are well established and include nanopore sequencing.

Other aspects of the invention provide a kit for use in a method of cleaving nucleic acid as described herein. The kit may comprise a bifunctional probe having the formula:

C-L-B
where C is a cleavage moiety, L is a linker and B is a binding moiety that covalently binds to a nucleic acid molecule.

Suitable bifunctional probes are described above.

The kit may further comprise zinc or copper, such as a copper (I) salt or a copper (II) salt and optionally a reducing agent. Suitable copper salts and reducing agents are described above.

In some embodiments, a kit may be useful in a method of determining the modification of nucleic acid in a cell. The kit may further comprise a co-factor analogue precursor or a co-factor analogue. Suitable co-factor analogue precursors and co-factor analogues are described above.

The kit may further comprise one or more cells, such as mammalian, preferably human cells, for example for use as controls. Suitable cells may include cells in which a nucleic acid modification enzyme has been inactivated. In some embodiments, a kit may comprise a pair of isogenic cells, a first cell in which a nucleic acid modification enzyme has been inactivated and a second cell in which the nucleic acid modification enzyme has not been inactivated.

The kit may further comprise DNAse and/or RNAse inhibitors. Suitable inhibitors are available from commercial sources.

The kit may further comprise control nucleic acids. For example, the kit may comprise positive controls that are subjected to modification and cleavage as described herein and/or negative controls that are not subjected to modification and cleavage as described herein.

The kit may further comprise nucleic acid primers for the amplification of validated genomic loci to be analysed for modification as described herein. Suitable primers may be generated by standard techniques or obtained from commercial suppliers.

A kit may further comprise components such as apparatus for sample collection, sample tubes, holders, trays, racks, dishes, plates, and other sample handling containers (such components generally being sterile), instructions to the kit user, solutions or other chemical reagents, such as DNA and/or RNA isolation and purification reagents, and other reagents required for the method, such as buffer solutions, sequencing and other reagents, and samples to be used for standardization, normalization, and/or control samples.

Other aspects of the invention provide the use of a bifunctional probe as described herein in a method of cleaving a nucleic acid molecule. Suitable methods of cleaving a nucleic acid molecule are described above.

Other aspects and embodiments of the invention provide the aspects and embodiments described above with the term “comprising” replaced by the term “consisting of” and the aspects and embodiments described above with the term “comprising” replaced by the term “consisting essentially of”.

It is to be understood that the application discloses all combinations of any of the above aspects and embodiments described above with each other, unless the context demands otherwise. Similarly, the application discloses all combinations of the preferred and/or optional features either singly or together with any of the other aspects, unless the context demands otherwise.

Modifications of the above embodiments, further embodiments and modifications thereof will be apparent to the skilled person on reading this disclosure, and as such, these are within the scope of the present invention.

All documents and sequence database entries mentioned in this specification are incorporated herein by reference in their entirety for all purposes.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

General Experimental Protocols

In Vitro Click-Degrader Functionalisation Reactions

In a standard reaction, CuSO₄ (final concentration 1.0 mM), THPTA (3.0 mM), click-degrader (2.0 mM) and the RNA oligo (200 μM) were added to pH 7.5 or pH 3 HEPES (20 mM) buffer supplemented with 10 mM MgCl₂ and 100 mM KCl. CuAAC was initiated by adding NaAsc (50 mM). The reaction mixture was then incubated at 37° C. Reactions to generate calibration curves were quenched with EDTA (12 mM) 20 min after adding NaAsc to allow complete functionalisation of the RNA oligo. Reactions involving pre-quenching were quenched with EDTA (12 mM) or bathocuproinedisulfonic acid (BCS) (3 mM) 20 min after adding NaAsc. After quenching the reaction mixtures were analysed using LCMS. Identities of RNA species present were determined by their mass (Table 1). For non-standard reactions the conditions are specified in the text.

In Vitro Copper-Free Click-Degrader Functionalisation Reactions

In a standard reaction, N-azidoacetylmannosamine (ManNAz, Sigma) (500 μM) or N-acetylmannosamine (ManNAc) was added to DBCO click degrader (500 μM) in DMEM+Glutamax cell media (final 4% DMSO). The reaction mixture was then incubated at 37° C. for 30 min. Reactions were quenched by addition of triphenylphosphine (2 mM) and analysed using HPLC-LCMS to generate chromatogram showing reactions completion. For non-standard reactions the conditions are specified in the text.

LCMS Analysis of Click Reactions

Small molecules were analysed using a Xevo G2-S TOF mass spectrometer coupled to an Acquity UPLC system using an Acquity UPLC BEH C18 1.7 μm column. The system utilises electronspray (ESI) ionisation. Two mobile phases were used—0.1% FA in H₂O and 0.1% FA in MeCN, with a flow rate of 0.200 mL/min. Calibration curves for the small molecules were based either on A₂₆₀ signals. Total mass spectra were reconstructed from the ion series using the MaxEnt algorithm preinstalled on MassLynx software (v. 4.1 from Waters) according to the manufacturer's instructions.

LCMS Analysis of Oligonucleotides

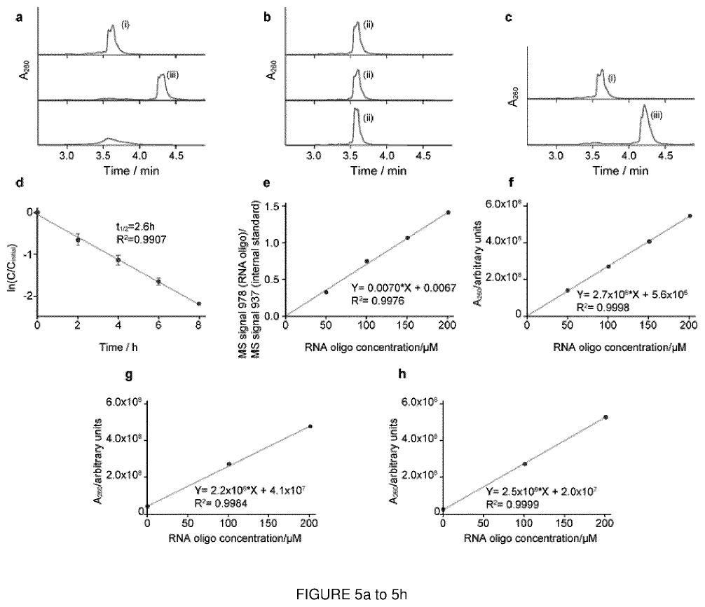

Oligomers were analysed using a Xevo G2-S TOF mass spectrometer coupled to an Acquity UPLC system using an Acquity UPLC BEH C18 1.7 μm column. The system utilises electronspray (ESI) ionisation. Two mobile phases were used—16.3 mM TEA, 400 mM HFIP in H2O and 16.3 mM TEA, 400 mM HFIP in 80:20 v/v MeCN and H₂O, with a flow rate of 0.200 mL/min. Calibration curves for the RNA species were based either on A₂₆₀ or intensities of specified negative m/z signals (FIG. 5e to h). Intensities of integrated peaks were calculated using native modules of KNIME software platform (33). Total mass spectra were reconstructed from the ion series using the MaxEnt algorithm preinstalled on MassLynx software (v. 4.1 from Waters) according to the manufacturer's instructions. To obtain the negative ion series described, the oligomer peak in the chromatogram was selected for integration and further analysis.

Cell Culture

MOLM13 cells were cultured in RPM11640 (Gibco) supplemented with 10% v/v FBS and 1% v/v penicillin/streptomycin/L-glutamine. 293T cells were cultured in DMEM (Gibco) supplemented with 10% v/v FBS and 1% v/v penicillin/streptomycin/L-glutamine.

Cell Culture (for acCLICK-Seq)

MOLM13 cells were cultured in DMEM+GlutaMAX media (Gibco) supplemented with 10% v/v FBS and 1% v/v penicillin/streptomycin/L-glutamine. All cells were grown in T75 or T175 CELLSTARR Standard Culture Flasks with standard screw cap red at 37° C. and 5% CO₂. Cells were maintained at >85% viability and were passaged every three days (or as needed). All cells were authenticated using short tandem repeat (STR) profiling. All cells were tested negative for mycoplasma contamination. Cells were only seeded for experiments at >80% viability.

Cell Culture (for glycoCLICK-Seq)

HeLa cells were cultured in DMEM+GlutaMAX media (Gibco) supplemented with 10% v/v FBS and 1% v/v penicillin/streptomycin/L-glutamine. All cells were grown in T75 or T175 CELLSTARR Standard Culture Flasks with standard screw cap red at 37° C. and 5% CO₂. Cells were maintained at >85% viability and were passaged every three days (or as needed). All cells were authenticated using short tandem repeat (STR) profiling. All cells were tested negative for mycoplasma contamination. Cells were only seeded for experiments at >80% viability.

Lentiviral Vector Production, Infection and Transfection

For virus production, 293T cells were transfected with lentiviral vector pLKO.1 together with the packaging plasmids PAX2 and VSVg at a 1:1.5:0.5 ratio. Supernatant was harvested 48 and 72 h after transfection. 1×10⁶ cells and viral supernatant were mixed in 2 mL culture medium supplemented with 8 μg ml⁻¹ polybrene (Millipore), followed by spinfection (60 min, 900 g, 32° C.) and further incubated overnight at 37° C. The medium was refreshed on the following day and the transduced cells were further cultured.

Generation of Conditional Knock-Down and Intronic Deletion-Containing Cells

MOLM-13 cells were transfected using Lipofectamine 2000 reagent (Invitrogen) according to the manufacturer's instructions using pLKO-TETon-Puro lentiviral vectors expressing shRNAs against the coding sequence of human METTL3, METTL16 or a scrambled control. 24 h after spinfection, the cells were replated in fresh medium containing 1 μg ml⁻¹ of puromycin and kept in selection medium for 7 days. Anti-METTL3 and scrambled shRNAs were induced by treating the cells with 200 ng mL¹ tetracycline for 3 days, anti-METTL16—by identical treatment for 2 days.

gRNA assays were performed using dual gRNA vectors as reported previously (34). Viral supernatants were collected 48 h after transfection. All transfections and viral collections were performed in 15-cm plates was performed as mentioned below. For virus production, 5 μg of the above plasmids and 5 μg psi-Eco packaging vector were transfected dropwise into the 293T cells using 47.5 μL TransIT LT1 (Mirus) and 600 μL Opti-MEM (Invitrogen). The resulting viral supernatant was harvested and transduction of cells was performed in 6-well plates. After transduction, transduced cells were sorted for BFP (for gRNA). The gRNA sequences are listed in Table 3.

Cellular RNA Degradation Reactions (Slick-Seq)

MOLM13 cells were suspended in methionine-free RPMI-1640 media (Gibco) supplemented with 10% v/v FBS and 1% v/v penicillin/streptomycin/L-glutamine at a density of 1,000,000 cells mL⁻¹. The cells were incubated for 30 min at 37° C. followed by addition of PropSeMet at a final concentration of 150 μM. Treated cells were incubated for further 16 h at 37° C. Aqueous solutions of premixed CuSO₄ and THPTA were added at final concentrations of 100 μM and 300 μM, respectively, followed by the click-degrader 1 at 400 μM and NaAsc at 5 mM. Treated cells were incubated for 10 min at 37° C. and resuspended in complete RPMI-1640 medium. Afterwards, the cells were again incubated at 37° C. and harvested after 5 h for RNA extraction.

Cellular RNA Degradation Reactions (acCLICK-Seq)

MOLM13 cells were suspended RPMI-1640 media (Gibco) supplemented with 10% v/v FBS and 1% v/v penicillin/streptomycin/L-glutamine at a density of 1,000,000 cells mL⁻¹. The cells were incubated for 30 min at 37° C. followed by addition of ethyl-3-butynoate at a final concentration of 200 μM. Treated cells were incubated for further 24 h at 37° C. Aqueous solutions of premixed CuSO₄ and THPTA were added at final concentrations of 100 μM and 300 μM, respectively, followed by the click-degrader 1 at 400 μM and NaAsc at 5 mM. Treated cells were incubated for 10 min at 37° C. and resuspended in RPMI-1640 medium. Afterwards, the cells were again incubated at 37° C. and harvested after 30 min for RNA extraction.

Cellular RNA Degradation Reactions (GlycoCLICK-Seq)

HeLa cells were suspended in DMEM+GlutaMAX media (Gibco) supplemented with 10% v/v FBS and 1% v/v penicillin/streptomycin/L-glutamine at a density of 1,000,000 cells mL⁻¹. The cells were incubated for 30 min at 37° C. followed by addition of azide sugars N-azidoacetylmannosamine-tetraacetylated (Ac₄ManNAz, Sigma), or 1,3,4,6-aetra-O-acetyl-N-azidoacetylglucosamine (Ac₄GlcNAz, Carbosynth), or 6-azidofucose-tetraacetylated (Ac₄FucAz, as synthesized in FIG. 13d) at a final concentration of 100 μM. Treated cells were incubated for further 24 h at 37° C. The cells were resuspended in media with DBCO-deg at a final concentration of 250 μM. Treated cells were incubated for 1 h at 37° C. and cleaved by addition of 0.25% Trypsin+0.02% EDTA, resuspended in complete DMEM medium, and harvested for RNA extraction.

m⁶A RNA Immunoprecipitation

Total RNA was isolated from MOLM-13 control, Δintronic, METTL3-KD or METTL16-KD cells (two independent biological replicates for each shRNA) eight days after doxycycline administration using the RNAeasy midi kit (Qiagen). Successively polyA enriched RNA was purified from 300 μg total RNA using the NEBNext Poly(A) mRNA Magnetic Isolation Module (New England Biolabs). 500 ng of polyA+purified RNA were used for each immunoprecipitation reaction. m⁶A RNA immunoprecipitation was performed using the Magna MeRIP m⁶A kit (Millipore) according to the manufacturer's instructions. Immunoprecipitated RNA was analysed via RT-qPCR.

RNA Extraction, Reverse Transcription and RT-qPCR

Total RNA was extracted from pelleted cells using RNAeasy Mini Kit (Qiagen) according to the manufacturer's instructions. 20 μg of total RNA was enriched for polyA-containing sequences using Dynabeads mRNA purification kit (Invitrogen) according to the manufacturer's instructions. 1 μg of total RNA and all of polyA-enriched RNA from the previous step were retrotranscribed using SuperScript Vilo Master Mix (Invitrogen) according the to manufacturer's instructions. Levels of specific RNAs were measured using fast mode of StepOnePlus Real-Time PCR System (Applied Biosystems) and Fast SYBR Green Master Mix (Applied Biosystems) according to the manufacturer's instructions. For reactions where total RNA was used, RNA levels were normalised to 18S subunit of the ribosome. For reactions where polyA-enriched RNA was used, RNA levels were normalised to RPL32 mRNA. These housekeeping genes were chosen as their levels fluctuated the least under various conditions tested. Primer sequences are listed in Table 2.

RNA extraction, reverse transcription and RT-qPCR (for acCLICK-seq and glycoCLICK-seq) Total RNA was extracted from pelleted cells using Qiazol (Qiagen) according to the manufacturer's instructions. 1 μg of total RNA from the Qiazol extraction step were retrotranscribed using SuperScript Vilo Master Mix (Invitrogen) according the to manufacturer's instructions. Levels of specific RNAs were measured using fast mode of QuantStudio 3 Real-Time PCR System (Applied Biosystems) and PowerUp™ SYBR Green Master Mix (Applied Biosystems) according to the manufacturer's instructions. In the qPCR analysis RNA levels were normalised to the 5S gene (acCLICK-seq) or the GAPDH gene (glycoCLICK-seq). These housekeeping gene was chosen as their levels fluctuated the least under various conditions tested. Primer sequences are listed in Table 2.

Statistical Analysis

General statistical analyses were carried out using a one-sided (in cases where we were interested only in a decrease of signal in the treated group) or two-sided Student's t-test at a confidence interval of 95%.

Protein Extraction and Immunoblotting

The cells were lysed in whole-cell lysis buffer (50 mM Tris-HCl, pH 8, 150 mM NaCl, 0.1% NP-40 and 1 mM EDTA) supplemented with 1 mM DTT, protease inhibitors (Sigma) and phosphatase inhibitors (Sigma). Protein quantities were estimated with Bradford assays (Bio-Rad). The protein samples were supplemented with SDS-PAGE sample buffer and DTT was added to each sample. 10-40 μg of protein were separated on a 4-12% Bis-Tris SDS-PAGE gel (Invitrogen) with a same amount of protein added to each track of a gel, and blotted onto polyvinylidene difluoride membranes (Millipore). Visualisation was performed using LumiGLO Chemiluminescent Substrate (KPL, 54-61-00) and X-ray film (GE Healthcare). The following antibodies were used: anti-METTL3 from Bethyl Laboratories (A301-568A), anti-METTL16 from Abcam (ab185990), anti-beta Actin from Abcam (ab8227), goat anti-rabbit IgG H&L (HRP) from Abcam (ab205718).

Bioinformatic Analysis

Total RNA was extracted from pelleted cells using RNAeasy Mini Kit (Qiagen) according to the manufacturer's instructions. RNAseq data from all experiments consisting of 75 bp paired-end Illumina reads were mapped to the human genome assembly GRCh38 by STAR 2.7.1b, using arguments “—outSAMunmapped Within KeepPairs—outFilterntronMotifs RemoveNoncanonicalUnannotated—chimSegmentMin 0—chimJunctionOverhangMin 20”. BigWig files were produced by deepTools 3.3.2 bamCoverage using RPKM normalization. Exonic reads were removed using the intersect function in Bedtools v2.29.0 and exon regions derived from the Ensembl GRCh38 Version 93. Reads on the forward strand were extracted using Samtools 1.9,⁴⁶ with “view-f 128—F 16” and “-f 80” and merged into one file. Reads on the reverse strand were extracted using “view-f 144” and “-f 64—F 16” and merged. Broad peaks were called by MACS2 2.1.4, using WT as treatment and METTL16/METTL3 as control, with arguments “callpeak—broad—extsize=300—keep-dup 20—nomodel-g hs—broad-cutoff 0.9—max-gap 500”. Intersection of peaks between the two replicates was taken. Peaks that show reduced signals in Slick-Seq compared to WT were selected as the final set.

To search for motifs, DNA sequences of peaks were extracted from the GRCh38 genomic FASTA file. Motifs were discovered by MEME-chip 5.0.5 using parameters “-meme-nmotifs 30”. For gene quantification, exonic regions were obtained from Ensembl GRCh38 version 93. Number of reads in each exon was calculated by customised code and normalised by RPKM. To counter the 3′ bias, the level of the most 3′ exon was used as expression of the gene.

m⁶A sites were analysed by first mapping Reads from the miCLIP experiments to the GRCh38 assembly using STAR 2.7.1 a with arguments “—outFilterMismatchNoverReadLmax 0.05—alignintronMax 0”. Forward and reverse genomic-coverage tracks were subsequently produced by the bamCoverage function in deepTools 3.3.2 with arguments “—normalizeUsing None-filterRNAstrand forward” and “—normalizeUsing None-filterRNAstrand reverse” respectively. Regions with non-zero coverage were extracted by customized codes. Sites called from 4 miCLIP runs were merged into a union set and the ones also present in the input data were removed.

One Pot Mixed RNA Experiment

Propargylated and non-propargylated RNA (100 μM each) in HEPES pH 7.4 buffer supplemented with KCl (100 mM) and MgCl₂ (10 mM) was treated with click-degrader 1 (5 mM), CuSO₄ complexed with THPTA (1 mM and 3.6 μM), the click reaction was triggered by adding NaAsc (50 mM). After 14 hours the reaction was quenched with EDTA (12 mM). The effect on RNA was analysed via LCMS at t=0 and 14 h.

Evaluation of Other Divalent Metal Ions Experiment

RNA oligomer (200 μM) in HEPES pH 7.4 buffer supplemented with KCl (100 mM) and MgCl₂ (10 mM) was treated with click-degrader 1 (400 μM), CuSO₄ complexed with THPTA (200 μM and 720 μM), the click reaction was triggered by adding NaAsc (5 mM). After 20 minutes, copper was quenched with bathocuproinedisulfonic acid (300 μM) and CuSO₄, FeSO₄ or ZnCl₂ (1 mM) were added. RNA was incubated for 14 hours and the reaction was quenched with EDTA (12 mM). The effect on RNA was analysed via LCMS at t=0 and 14 h.

### Chemical Synthesis

General Considerations

Chemicals were purchased from Fisher Chemicals, Sigma-Aldrich, Alfa-Aesar, Fluorochem or BioSynth and used without further purification. All solvents were commercially available grade. All non-aqueous reactions were performed in oven-dried glassware under a nitrogen atmosphere unless otherwise stated. Nitrogen gas was pre-dried via passage through calcium chloride. Reaction vessels were heated using thermostatically controlled DrySyn blocks filled with sand with the liquid level of the flask below that of the heating block. Reaction temperatures refer to the thermostat set point. A reaction temperature of 0° C. refers to an external ice/water slurry cooling bath. All reagents were purchased from commercial sources and used without further purification unless otherwise stated. DCM, MeOH and MeCN were distilled from calcium hydride. THE and Et₂O were pre-dried over sodium wire then distilled from calcium hydride and lithium aluminium hydride. Petroleum ether, n-hexane and EtOAc were distilled on site. ‘Petroleum ether’ refers to the distillate of petroleum ether collected between 40-60° C. Water used experimentally was deionised and prepared on site.

Flash column chromatography was performed using Merck silica gel 60. Analytical thin layer chromatography was performed using Merck Silica gel 60 F254 and visualised by UV (254 nm), by staining with a KMnO₄ or (NH₄)₄Ce(SO₄)₄ solution. NMR spectra were recorded on a 400 MHz AVIII HD Smart Probe Spectrometer or a 600 MHz Avance 600 BBI Spectrometer. HPLC analysis and purification were carried out on ThermoFisher Scientific Ultimate 3000 HPLC system, using NUCLEOSIL 100-5 C18 semi-preparative column. mqH₂O with 0.1% formic acid (A) and HPLC grade MeCN (B) were used as the mobile phases, with flow rate of 3 mL/min.

For compounds 17 to 21, all non-aqueous reactions were performed in oven-dried glassware under an argon atmosphere unless otherwise stated. Argon gas was pre-dried via passage through calcium chloride. Reaction vessels were heated using thermostatically controlled DrySyn blocks filled with sand with the liquid level of the ask below that of the heating block.

The general chemicals were purchased from commercial sources and used without further purification process unless stated otherwise. DCM, THF and Et2O were purified either according to the method of Grubbs and Pangborn or by distillation under an inert atmosphere (DCM, MeOH and MeCN were distilled from calcium hydride. THF and Et2O were pre-dried over sodium wire then distilled from calcium hydride and lithium aluminium hydride). EtOAc was distilled on site. Water used experimentally was deionised and prepared on site.

Flash column chromatography was performed using silica gel 60 Å (40-63 μm) from Material Harvest. Analytical thin layer chromatography was performed using Merck Silica gel 60 F254 1 mm glass plates and visualised by UV (254 nm) or by staining with an indicated solution prepared by known procedures.

NMR spectra were recorded on Bruker 400-Avance III HD, 600 MHz Avance 600 BBI spectrometers, 400 MHz Neo Prodigy, 400-QNP Cryoprobe or 500-DCH Cryoprobe spectrometers. Chemical shifts are reported in parts per million (ppm) and the spectra are calibrated to the residual solvent peak (¹H NMR: CDCl3 δ 7.26 ppm, 44 DMSO δ 2.50 ppm; ¹³C NMR: CDCl₃ δ 77.16 ppm, DMSO δ 39.52 ppm). Multiplicities are described as s (singlet), d (doublet), t (triplet), q (quartet), m (multiplet), dd (double doublet) etc. Coupling constants (J) are reported in hertz (Hz). TopSpin software version 3.5 was used for signal processing. The center of each peak is reported except for multiplet signals where a range of ppm values are given. Structural assignments are made with the aid of COSY, HSQC and HMBC experiments, performed by the NMR Spectrometry Service, University of Cambridge.

High resolution mass spectra were performed by the Mass Spectrometry Service, Department of Chemistry, University of Cambridge using a Waters LCT Premier or a Waters Xevo G2-Spectrometer using electrospray (ESI) method.

Overview of Synthesis

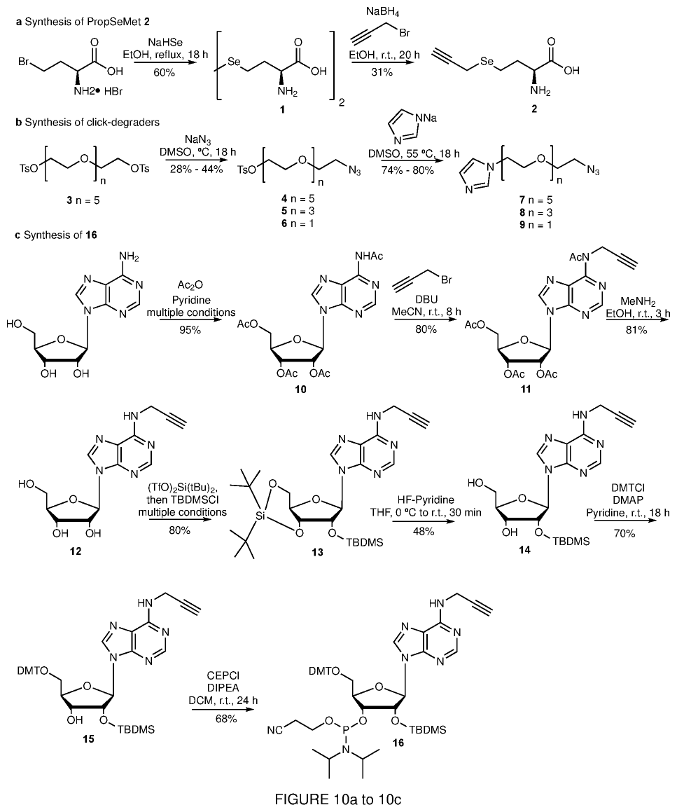

A schematic overview of the chemical synthesis of PropSeMet (2), click-degradors (7, 8, and 9) and orthogonally-protected N⁶-propargyladenosine (16) is shown in FIG. 10.

Selenohomocystine (1)

100-Mesh selenium powder (1.8 g, 23 mmol) was suspended in 35 mL absolute ethanol and cooled to 0° C. Sodium borohydride (0.87 g, 23 mmol) was added and the mixture was refluxed for 2 h (the solution became deep maroon in colour). Into the reaction vessel was added α-amino-4-bromobutanoic acid hydrobromide (3.00 g, 12 mmol), resulting in a rapid formation of an opaque yellow mixture. The reaction was stirred under reflux for 18 h and then quenched with 5 mL 2 M HCl. After removing bulk solvent by rotary evaporation, the resulting residue was mixed with 15 mL 5% HCl. This aqueous solution was then washed three times with 40 mL Et₂O and subjected to vacuum filtration to remove insoluble materials. A yellow solid was obtained after removing aqueous solvent in vacuo. This semi-crude product was then dissolved in 8 mL 1 M HCl and purified over Dowex® 50WX8 ion exchange resin. Activated resin (15 ml) was washed with 100 mL H₂O, followed by the loading of semi-crude product. The resin was then washed with an additional 100 mL H₂O to remove unbound impurities. Product was eluted from the resin using a 5% ammonium hydroxide solution. Fractions containing the desired product (bright yellow solution) were combined. After removing the bulk solvent, the resulting yellow solid was dried in vacuo to afford a yellow powder (1.3 g, 3.5 mmol, 60%). ¹H NMR (400 MHz, D20 with 0.1% TFA) 5 4.01 (t, 2H), 2.95 (td, 4H), 2.21-2.43 (m, 4H). HRMS [+scan]: calculated m/z C₈H₁₇N₂O₄Se₂ 364.9513; observed 364.9514.

PropSeMet (Propargylic-L-Selenomethionine) (2)

1 (119 mg, 0.33 mmol) was dissolved in EtOH (20 mL) under a N₂ atmosphere, NaBH₄ (124 mg, 3.3 mmol) was added and the solution was stirred for 15 min at room temperature. NaHCO₃(150 mg, 1.8 mmol) and 80% propargyl bromide in toluene (245 mg, 2.1 mmol) were added and the mixture was stirred for further 20 h at room temperature. The solvent was removed in vacuo and the crude product was dissolved in 5 mL mqH₂O supplemented with 1% TFA. The solution was adjusted to pH=3 using HCl, the product was purified via HPLC, fractions containing the product were lyophilised resulting in a white powder (45 mg, 0.21 mmol, 31%). ¹H NMR (400 MHz, D₂O): δ 3.78 (t, 1H), 3.25 (d, 2H), 2.79 (t, 2H), 2.60 (t, 1H), 2.17-2.37 (m, 2H). ¹³C NMR (100 MHz, D20): δ_{C} 173.1, 81.5, 72.4, 54.2, 31.3, 19.1, 6.4. HRMS [+scan]: calculated m/z C₇H₁₂NO₂Se 222.0024; observed 222.0028.

### Hexaethylene glycol di(p-toluenesulfonate) (3)

Hexaethylene glycol (2.0 g, 7.1 mmol) was dissolved in DCM and cooled to 0° C., followed by addition of p-toluenesulfonyl chloride (3.0 g, 14 mmol) and KOH (3.2 g, 57 mmol). The solution was stirred for 3 h at 0° C. and 30 min at room temperature and quenched with H₂O (20 ml). The product was extracted with DCM (3×20 ml), combined organic phases were washed with brine (20 ml) and dried (MgSO₄). Organic solvents were removed in vacuo, the product was obtained as a colourless oil (3.9 g, 6.5 mmol, 92%). ¹H NMR (400 MHz, CDCl₃): δ 7.78 (d, 4H), 7.33 (d, 4H), 4.14 (t, 4H), 3.67 (br tr, 4H, 3.60 (br s, 8H), 3.57 (br s, 8H), 2.43 (br s, 6H). ¹³C NMR (100 MHz, CDCl₃): δ_{C} 144.8, 133.0, 129.8, 128.0, 70.5-70.7 (Multiple PEG peaks), 69.3, 68.7, 21.7. HRMS [+scan]: calculated m/z for C₂₆H₃₉O₁₁S₂ 591.1934; observed 591.1931.

### Hexaethylene glycol p-toluenesulfonate azide (4)

3 (2.0 g, 3.4 mmol) was dissolved in anhydrous DMF (10 ml). Sodium azide (242 mg, 3.7 mmol) was added, the mixture was placed under N₂ and stirred for 18 h at 55° C. Solvent was removed in vacuo, products were purified via flash column chromatography (gradient 1:1 Pet. Ether:AcOEt to AcOEt). The product was obtained as a pale-yellow oil (440 mg, 0.95 mmol, 28%). ¹H NMR (400 MHz, CDCl₃) δ 7.82 (d, 2H), 7.36 (d, 2H), 4.18 (t, 2H), 3.59-3.73 (20H, PEG), 3.41 (t, 2H), 2.47 (s, 3H). ¹³C NMR (100 MHz, CDCl₃) δ_{C} 144.8, 133.0, 129.3, 128.0, 70.5-70.8 (Multiple PEG peaks), 70.0, 69.3, 68.7, 50.7, 21.7. HRMS [+scan]: calculated m/z for C₁₉H₃₁N₃NaO₈S 484.1730; observed 484.1723.

Tetraethylene glycol p-toluenesulfonate azide (5)

Tetraethylene glycol di(p-toluenesulfonate) (2.7 g, 5.4 mmol) was dissolved in anhydrous DMF (10 ml). Sodium azide (355 mg, 5.4 mmol) was added and the mixture was placed under N₂ and stirred for 18 h at 55° C. Solvent was removed in vacuo, products were purified via flash column chromatography (3:1 Pet. Ether:AcOEt to 1:1 Pet. Ether:AcOEt). The product was obtained as a colourless oil (798 mg, 2.1 mmol, 39%). ¹H NMR (400 MHz, CDCl₃) δ 7.82 (d, 2H), 7.37 (d, 2H), 4.19 (t, 1H), 3.60-3.73 (12H, PEG), 3.40 (t, 2H), 2.47 (s, 3H). HRMS [+scan]: calculated m/z for C₁₅H₂₃N₃NaO₆S 396.1205; observed 396.1207.

### Diethylene glycol p-toluenesulfonate azide (6)

Diethylene glycol di(p-toluenesulfonate) (2.3 g, 5.4 mmol) was dissolved in anhydrous DMF (10 ml). Sodium azide (353 mg, 5.4 mmol) was added and the mixture was placed under N₂ and stirred for 18 h at 55° C. Solvent was removed in vacuo, products were purified via flash column chromatography (3:1 Pet. Ether:AcOEt to 1:1 Pet. Ether:AcOEt). The product was obtained as a colourless oil (681 mg, 2.4 mmol, 44%). ¹H NMR (400 MHz, CDCl₃) δ 7.80 (d, 2H), 7.35 (d, 2H), 4.17 (t, 2), 3.70 (t, 2H), 3.61 (t, 2H), 3.32 (t, 2H), 2.45 (s, 3H). HRMS [+scan]: calculated m/z for C₁₁H₁₅N₃NaO₄S 308.0681; observed 308.0670.

### Click-degrader 1 (hexaethylene glycol imidazolate azide) (7)

Imidazole (44 mg, 0.65 mmol) and NaH (60% dispersion in mineral oil, 26 mg, 0.65 mmol) were suspended in anhydrous DMF (2 ml) at 0° C. The mixture was placed under N₂ atmosphere, allowed to warm to room temperature and stirred for 30 min. 4 (250 mg, 0.54 mmol) was dissolved in anhydrous DMF (3 ml) and the resulting solution was added to the first mixture. It was then stirred for 20 h at 55° C. Solvent was then removed in vacuo and the resulting residue was purified via flash chromatography (dry loading, gradient EtOAC to 9:1 EtOAc: MeOH). The product was obtained as a colourless oil (154 mg, 0.43 mmol, 80%). ¹H NMR (400 MHz, CDCl₃) 5H 7.52 (s, 1H), 7.02 (s, 1H), 6.98 (s, 1H), 4.09 (t, 2H), 3.72 (t, 2H), 3.55-3.78 (18H, PEG), 3.36 (t, 2H). ¹³C NMR (100 MHz, CDCl₃) δ_{C} 137.6, 129.3, 119.4, 70.6-70.7 (Multiple PEG peaks), 70.0, 50.7, 47.0. HRMS [+scan]: calculated m/z for C₁₅H₂₈N₅O₅ 358.2090; observed 358.2084.

### Click-degrader 2 (Tetraethylene glycol imidazolate azide) (8)

Imidazole (18 mg, 0.27 mmol) and NaH (60% dispersion in mineral oil, 12 mg, 0.27 mmol) were suspended in anhydrous DMF (1 ml) at 0° C. The mixture was placed under N₂ atmosphere, allowed to warm to room temperature and stirred for 30 min. 5 (100 mg, 0.27 mmol) was dissolved in anhydrous DMF (1 ml) and the resulting solution was added to the first mixture. It was then stirred for 20 h at 55° C. Solvent was then removed in vacuo and the resulting residue was purified via flash chromatography (dry loading, gradient EtOAC to 9:1 EtOAc: MeOH). The product was obtained as a colourless oil (54 mg, 0.20 mmol, 74%). ¹H NMR (400 MHz, CDCl₃) δ 7.55 (s, 1H), 7.05 (s, 1H), 7.05 (s, 1H), 4.12 (t, 2H), 3.75 (t, 2H), 3.60-3.71 (1OH, PEG), 3.39 (t, 2H). ¹³C NMR (100 MHz, CDCl₃) δ_{C} 137.6, 129.2, 119.4, 70.5-70.7 (multiple PEG peaks), 70.0, 50.7, 47.1. HRMS [+scan]: calculated m/z for C₁₁H₁₉N₅O₃ 270.1566; observed 270.1582.

### Click-degrader 3 (Diethylene glycol imidazolate azide) (9)

Imidazole (93 mg, 1.4 mmol) and NaH (60% dispersion in mineral oil, 55.0 mg, 1.4 mmol) were suspended in anhydrous DMF (2 ml) at 0° C. The mixture was placed under N₂ atmosphere, allowed to warm to room temperature and stirred for 30 min. 6 (300 mg, 1.1 mmol) was dissolved in anhydrous DMF (3 mL) and the resulting solution was added to the first mixture. It was then stirred for 20 h at 55° C. Solvent was then removed in vacuo and the resulting residue was purified via flash chromatography (dry loading, gradient EtOAC to 9:1 EtOAc: MeOH). The product was obtained as a colourless oil (139 mg, 0.77 mmol, 77%). ¹H NMR (400 MHz, CDCl₃) δ 7.53 (s, 1H), 7.06 (s, 1H), 6.99 (s, 1H), 4.14 (t, 2H), 3.75 (t, 2H), 3.60 (t, 2H), 3.36 (t, 2H). ¹³C NMR (100 MHz, CDCl₃) δ_{C} 137.5, 129.4, 119.4, 70.6, 70.1, 50.7, 47.1. HRMS [+scan]: calculated m/z for C₁₁H₁₉N₅O₃ 182.1042; observed 182.1041.

### N⁶-Acetyl-2;3′,5′-tri-O-acetyladenosine (10)

To adenosine (2.0 g, 7.5 mmol) were added pyridine (15 mL), and Ac₂O (7.0 mL, 74 mmol), the resulting white cloudy mixture was stirred at room temperature overnight. The resulting solution was heated at 55° C. overnight. The reaction was cooled down and quenched by addition of excess of EtOH. The solvent was evaporated in vacuo. To remove traces of pyridine the residue was co-evaporated successively with portions of EtOH. The resultant foam was dissolved in MeOH (20 mL); imidazole (0.3 g, 4.4 mmol) was added and the solution was stirred at room temperature overnight. The solution was diluted with DCM (150 mL) and washed with brine (4×40 mL). The organic layer was dried (MgSO₄) and the solvent was removed in vacuo to yield the product as a white foam (3.1 g, 7.1 mmol, 95%). ¹H NMR (CDCl₃, 400 MHz): δ 9.79 (br s, 1H), 8.66 (s, 1H,), 8.33 (s, 1H), 6.22 (d, 1H), 5.95 (dd, 1H), 5.65 (dd, 1H), 4.45-4.52 (m, 2H), 4.33 (dd, 1H), 2.59 (s, 3H), 2.12 (s, 3H), 2.05 (s, 3H), 2.04 (s, 3H).¹³C NMR (100 MHz, CDCl₃) δ_{C} 171.3, 170.4, 169.6, 169.4, 152.5, 151.2, 149.6, 141.9, 122.3, 86.3 80.3, 73.1, 70.6, 63.1, 25.7, 20.7, 20.5, 20.4. HRMS [+scan]: calculated m/z for C₁₈H₂₂N₅O₈ 436.1468; observed 436.1459.

### N⁶—Acetyl-2;3′,5′-tri-O-acetyl-N⁶-propargyladenosine (11)

To a stirred solution of 10 (3.1 g, 7.1 mmol) in anhydrous MeCN under Ar-atmosphere, DBU (3.2 mL, 21.4 mmol) and 80% propargyl bromide in toluene (2.0 mL, 18 mmol) were added at r.t. The resulting brown mixture was stirred at room temperature for 8 h. The mixture was diluted with 150 mL DCM and extracted with 50 mL 0.5 M HCl and three times with 100 mL brine. The organic layer was dried (MgSO₄) and the solvents were removed to yield the product as a brown foam (2.7 g, 5.7 mmol, 80.4%). ¹H NMR (CDCl₃, 400 MHz): δ 8.84 (s, 1H), 8.24 (s, 1H), 6.27 (d, J=5.1 Hz), 5.99 (dd, 1H), 5.70 (dd, 1H), 5,11 (dd, 2H), 4.45-4.54 (m, 2H), 4.37-4.45 (m, 1H) 2.40 (s, 3H), 2.18 (s, 3H), 2.15 (s, 3H), 2.12 (s, 3H), 2.02 (s, H). ¹³C NMR (100 MHz, CDCl₃) δ_{C} 170.9, 170.3, 169.6, 169.4, 152.6, 152.5, 152.2, 142.3, 126.9, 86.8, 80.4, 79.2, 73.1, 71.6, 70.5, 63.1, 36.4, 24.4, 20.8, 20.5, 20.4. HRMS [+scan]: calculated m/z for C₁₉H24N₅O₈ 450.1619; observed 450.1626.

### N⁶-Propargyladenosine (12)

11 (2.7 g, 5.7 mmol) was dissolved in 25 mL 8M MeNH₂ in EtOH under N₂ atmosphere. The solution was stirred for 3 h at room temperature. The solvents were removed, and the residue was dissolved in 180 mL EtOAc and 20 ml EtOH. The organic layer was extracted with 180 mL brine and the aqueous layer was washed with 10×100 mL of EtOAc. The organic layers were dried (MgSO₄) and the solvents were removed to yield a yellow solid. 100 mL EtOAc and 100 mL Et20 were added to the residue and the mixture was kept at 4° C. for 16 h. The white precipitate was collected by filtration and washed with Et20 to yield the product as a white powder (1.4 g, 4.6 mmol, 81%). ¹H NMR (400 MHz, DMSO-d₆) δ 8.40 (s, 1H), 8.29 (s, 1H), 8.23 (br s, 1H), 5.91 (d, 1H), 5.49 (d, 1H), 5.40 (dd, 1H), 5.23 (d, 1H), 4.61 (dd, 1H), 4.28 (dd, 1H), 3.98 (dd, 1H), 3.69 (m, 1H), 3.56 (m, 1H), 3.03 (br s, 1H). ¹³C NMR (100 MHz, DMSO-d₆) δ_{C} 154.4, 152.7, 149.3, 140.7, 120.4, 88.4, 86.3, 82.3, 74.0, 72.9, 71.0, 62.1, 29.6. HRMS [+scan]: calculated m/z for C₁₃H₁₅N₅O₄ 306.1202; observed 306.1246.

### 2′-O-(tert-butyldimethylsilyl)-3′,5′-O—(di-tert-butylsilylene)-N⁶-propargyladenosine (13)

12 (1.1 g, 3.7 mmol) was dissolved in anhydrous DMF (20 ml) and cooled to 0° C., followed by dropwise addition of di-tert-butylsilyl bis(trifluoromethane sulfonate) (1.4 ml, 4.1 mmol). Mixture was stirred for 30 min at 0° C., then 15 min at room temperature. Imidazole (1.0 g, 15 mmol) was then added, followed by TBDMSCI (1.1 g, 7.4 mmol). Mixture was stirred for 1 h at room temperature, then for 3 h at 60° C. Then most of the solvent was removed in vacuo and dissolved in 200 mL Et20 and washed three times with 40 mL H₂O. Organic layer was dried over MgSO₄ and filtered; solvent was removed in vacuo. Crude was purified via flash chromatography (3:1 petroleum ether: AcOEt). The product 1.7 g (3.0 mmol) of white powder were obtained, corresponding to yield of 80%. ¹H NMR (400 MHz, CDCl₃) δ 8.40 (s, 1H), 7.86 (s, 1H), 6.79 (t, 1H), 5.92 (s, 1H), 4.60 (s, 1H), 4.40-4.55 (m, 4H), 4.21 (dt, 1H), 4.03 (m, 1H), 2.24 (t, 1H), 1.65 (s, 3H), 1.07 (s, 9H), 1.03 (s, 9H), 0.92 (s, 9H), 0.16 (s, 3H), 0.14 (s, 3H). ¹³C NMR (100 MHz, CDCl₃) δ_{C} 154.1, 153.2, 148.7, 138.5, 120.6, 92.4, 80.3, 75.8, 75.5, 74.7, 71.3, 67.8, 27.5, 27.0, 25.9, 22.7, 20.3, 18.3 , −4.3, -5.0. HRMS [+scan]: calculated m/z for C₂₇H₄₆N₅O⁴Si₂ 560.3088; observed 560.3099.

### 2′-O-(tert-butyldimethylsilyl)-N⁶-propargyladenosine (14)

13 (1.23 g, 2.2 mmol) was dissolved in anhydrous THE (30 mL) and cooled to 0° C. HF-pyridine (1.2 mL) was diluted with pyridine (1.2 mL), added to the reaction mixture and allowed to war to room temperature, followed by stirring for 30 min. Reaction was then quenched with pyridine (2 mL) and DCM (60 mL). It was then washed with saturated NaHCO₃ solution (40 mL) and brine (2×40 mL). Organic layers were dried with MgSO₄ and solvents were removed in vacuo. Crude product was purified via column chromatography (dry loading, eluent gradient 5:1 petroleum ether: AcOEt to AcOEt). The product was obtained as a white powder (440 mg, 1.1 mmol, 48%). ¹H NMR (400 MHz, CDCl₃) δ 8.41 (s, 1H), 7.77 (s, 1H), 6.62 (d, 1H), 6.11 (br s, 1H), 5,75 (d, 1H), 5.14 (dd, 1H), 4.49 (s, 1H), 4.34-4.37 (m, 2H), 3.95 (d, 1H), 3.74 (t, 1H), 2.82 (s, 1H), 2.30 (t, 1H), 0.80 (s, 9H),−0.15, (s, 3H),−0.36 (s, 3H). HRMS [+scan]: calculated m/z for C₁₉H₃₀N₅O₄Si 420.2067; observed 420.2082.

### 2′-O-(tert-butyldimethylsilyl)-5′-O-(4,4′-dimethoxytrityl)-N⁶-proaprgyladenosine (15)

14 (410 mg, 0.98 mmol) was dissolved in anhydrous pyridine (10 mL), DMTCI (390 mg, 1.1 mmol) and DMAP (23 mg, 0.20 mmol) were added, mixture was placed under N₂ atmosphere and stirred overnight at room temperature for 18 h. The solvent was removed in vacuo and the residue was purified on a column (gradient, DCM+1% NEt₃ to 4:1 DCM:AcOEt+1% NEt₃). The product was obtained as a white powder (497 mg, 0.69 mmol, 70%). ¹H NMR (400 MHz, CDCl₃) δ 8.35 (s, 1H), 7.99 (s, 1H), 7.45 (d, 2H), 7.34 (d, 4H), 7.18-7.31 (m, 3H), 6.81 (d, 4H) 6.02 (d, 1H), 5.86 (br s, 1H), 4.99 (t, 1H), 4.48 (br s, 2H), 4.34 (m, 1H), 4.25 (m, 1H), 3.79 (s, 6H), 3.52 (dd, 1H), 3.38 (dd, 1H), 2.70 (d, 1H), 2.28 (t, 1H), 0.84 (s, 9H),−0.01, (s, 3H),−0.13 (s, 3H). HRMS [+scan]: calculated m/z for C₄₀H₄₇N₅NaO₆Si 744.3193; observed 744.3191.

### 2′-O-(tert-butyldimethylsilyl)-3′-O-(2-cyanoethyl-N,N-diisopropylphosphino)-5′-O-(4,4′-dimethoxytrityl)-N⁶-propargyladenosine (16)

15 (325 mg, 0.44 mmol) was dissolved in DCM (7 mL) and degassed, DIPEA (140 mL, 1.8 mmol) and CEPCI (140 mg, 1.3 mmol) were added to the mixture and stirred for 24 h at room temperature. The crude mixture was directly purified via a column (8:1 DCM:Acetone+1% NEt₃). The product was obtained as a white product (284 mg, 0.30 mmol, 68%). ¹H NMR (500 MHz, CDCl₃, reported for the major of the two diastereomers) 5 8.31 (s, 1H), 7.98 (s, 1H), 7.46 (d, 2H), 7.35 (d, 4H), 7.19-7.29 (m, 3H), 6.81 (d, 4H) 6.01 (d, 1H), 5.84 (br s, 1H), 5.07 (m, 1H), 4.47 (br s, 2H), 4.24-4.32 (m, 3H), 3.78 (s, 6H), 3.52-3.68 (dd, 5H), 3.31 (m, 1H), 2.65 (m, 2H), 2.29 (m, 2H), 1.15-1.21 (m, 12H), 0.76 (s, 9H),−0.05, (s, 3H),−0.21 (s, 3H). ³¹P NMR (162 MHz, CDCl₃) δ 149.0, 150.9. HRMS [+scan]: calculated m/z for C₄₉H₆₄N₇NaO₇PSi 944.4271; observed 944.4258.

### Ethyl-3-butynoate (17)

To a solution of but-3-ynoic acid (182 mg, 2.16 mmol) in ethanol (1.5 mL) was added sulfuric acid (57 μL) and the reaction mixture was set aside at room temperature for 10 days. The crude was dissolved in EtOAc (10 mL), washed with NaHCO₃(sat. soln., 10 mL) and dried over anhydrous MgSO₄ and concentrated in vacuo to yield 17 as a yellow oil (56 mg, 0.50 mmol, 23%). ¹H NMR (600 MHz, CDCl₃): δ (ppm)=4.24 (2H, q, J=8.0 Hz), 3.31 (2H, t, J=4.0 Hz), 2.22 (1H, t, J=4.0 Hz), 1.32 (3H, t, J=8.0 Hz). ¹³C NMR (150 MHz, CDCl₃): δ (ppm)=167.8, 75.8, 71.7, 61.7, 25.8, 14.1. HRMS-ESI (m/z): [M+H]⁺ calcd. for C₆H₉O₂, 113.0597; found 113.0610.

### Hexaethylene glycol mono(p-toluenesulfonate) (18)

To a solution of hexaethylene glycol (1.25 g, 4.43 mmol) in DCM (8.9 mL) was added TsCI (0.85 g, 4.43 mmol) and the reaction mixture was cooled down to 0° C. Anhydrous pyridine (0.70 mL, 8.86 mmol) was added and the mixture was stirred at r.t. for 2 h. The solvent was removed in vacuo and the crude was purified by column chromatography (EtOAc with a trace of DCM for charging then EtOAc: MeOH=20:1→15:1→10:1→6:1) to afford 2 as a clear oil (676 mg, 1.55 mmol, 35%). ¹H NMR (400 MHz, CDCl₃): δ (ppm)=7.80 (2H, dt, J=8.0 Hz), 7.35 (2H, d, J=8.0 Hz), 4.15 (2H, t, J=4.0 Hz), 3.69-3.58 (22H, m), 2.46 (3H, s). ¹³C NMR (100 MHz, CDCl₃): δ (ppm)=144.77, 133.00, 129.81, 127.97, 72.52, 70.71, 70.59, 70.55, 70.53, 70.51, 70.50, 70.29, 69.25, 68.67, 61.72, 21.63. HRMS-ESI (m/z): [M+H]⁺calcd. for C₁₉H₃₃O₉S, 437.1840; found 437.1843.

### Hexaethylene glycol monoimidazole (19)

To hexaethylne glycol mono(p-toluenesulfonate) 18 (153 mg, 0.35 mmol) was added imidazole (239 mg, 3.51 mmol) and the mixture was stirred at 110° C. for 20 h. The reaction mixture was dissolved in MeOH, the solvent was removed in vacuo and remaining imidazole was sublimed by using a Kugelrohr apparatus. Purification of the residue via column chromatography (DCM only→DCM:MeOH=15:1→10:1→5:1) yielded 19 as colorless oil (109 mg, 0.33 mmol, 93%). ¹H NMR (400 MHz, CDCl₃): δ (ppm)=7.54 (1 H, s), 7.02 (2H, d), 4.11 (2H, t), 3.75-3.71 (4H, m), 3.66-3.59 (18H, m). ¹³C NMR (100 MHz, CDCl₃): δ (ppm)=137.56, 129.18, 119.42, 72.69, 70.66, 70.62, 70.61, 70.57, 70.55, 70.53, 70.51, 70.31, 61.63, 47.03. HRMS-ESI (m/z): [M+H]⁺ calcd. for C₂₉H₄₀N₉OS, 333.2020; found 333.2021.

### Click degrader 4 (hexaethylene glycol imidazolate dibenzocyclooctyne) (20)

To a solution of 1,1′-carbonyldiimidazole (CDI) (49 mg, 0.30 mmol) dissolved in anhydrous THE (1.1 mL) was added 19 (50 mg, 0.15 mmol) and the reaction mixture was stirred at room temperature for 21 h. 3-(5H,6H-11,12-didehydrodibenzo[b,f]azocin-5-yl)-3-oxopropyl)amine (46 mg, 0.16 mmol) was then added and the reaction mixture was stirred at room temperature for 24 h. The solvent was removed in vacuo and the residue was purified by preparative TLC (DCM:MeOH=5:1) to give 20 (80 mg, 0.14 mmol, 90%) as a yellow oil. ¹H NMR (600 MHz, CDCl₃): δ (ppm)=7.68-7.67 (1H, m), 7.56 (1 H, s), 7.43-7.30 (7H, m), 7.06-7.05 (1H, m), 7.02-7.00 (1H, m), 5.36 (1H, br), 5.14 (1H, d, J=14.4 Hz), 4.16-4.14 (2Hm), 4.12 (2H, t, J=504 Hz), 3.75 (2H, quint, J=5.4 Hz), 3.70 (1H, d, J=14.4 Hz), 3.67-3.58 (1H, m), 3.30-3.17 (2H, m), 2.53 (1 H, ddd, J=16.8 Hz, 6.6 Hz, 3.2 Hz), 1.95 (1 H, ddd, J=16.8 Hz, 6.6 Hz, 3.2 Hz). ¹³C NMR (150 MHz, CDCl₃): δ (ppm)=171.9, 156.3, 151.1, 148.0, 137.5 132.0, 129.1, 129.0, 128.5, 128.3, 127.8, 127.2, 125.6, 22.9, 122.7, 119.5, 107.5, 70.7, 70.6, 70.6, 70.5, 70.5,70.5, 69.6, 63.8, 55.5, 53.4, 50.6, 47.1, 36.9, 34.9. HRMS-ESI (m/z): [M+H]⁺ calcd. for C₃₄H₄₃N₄O₈, 635.3075; found 635.3077.

### 1,2,3,4-tetra-O-acetyl-6-azido-6-deoxy-α-L-galactopyranoside (21) (35)

To a stirring solution of 6-azido-1,2,3,4-di-O-isopropylidene-6-deoxy-α-Lgalactopyranoside (250 mg, 0.88 mmol) in anhydrous DCM (5 mL) under an N₂ atmosphere was added trifluoroacetic acid:H2O (3:1, 4 mL). After stirring for 3 h at room temperature, the mixture was concentrated in vacuo. The resulting residue was dissolved in pyridine (3 mL) and acetic anhydride (1 mL) was added. The solution was stirred for 24 h, quenched with methanol (4 mL) and concentrated in vacuo. Purification by column chromatography (Petrol: Ethyl acetate=4:1→3:1→2:1) to give a colorless oil (112 mg, 34%). ¹H NMR (400 MHz, CDCl₃): δ (ppm)=6.39 (1 H, br s), 5.48 (1 H, br s), 5.33 (2H, br s), 4.24-4.23 (1 H, ddd), 3.47-3.42 (1H, dd), 3.24-3.19 (1H, dd), 2.18 (6H, d), 2.01 (6H, d). ¹³C NMR (150 MHz, CDCl₃): δ (ppm)=170.07, 170.05, 169.87, 168.86, 89.60, 70.11, 68.10, 67.34, 66.32, 50.29, 20.87, 20.61, 20.60, 20.52. HRMS-ESI (m/z): [M+H]⁺ calcd. for C₁₄H₁₉N₃O₉, 373.1121; found 373.1120.

Investigation of Slick-Seq RNA Degradation

To overcome numerous limitations pertinent to antibody-based RNA methylation methods, the present inventors have developed Slick-Seq, a small molecule-based transcriptome editing platform which hijacks endogenous RNA methylation pathways(9), leading to specific degradation of methylated RNAs (FIG. 1a). These effects are achieved exclusively through direct treatment of cultured cells with small molecules avoiding the complexed processing of RNA. In the first step of Slick-Seq, methionine surrogate PropSeMet is introduced to the media. Cells uptake the methionine surrogate and enzymatically transform it to SeAdoYn, a surrogate of the cofactor S-Adenosyl methionine (SAM). RNA methyltransferases (MTases) including METTL3 and METTL16 employ SeAdoYn as a cofactor instead of SAM, which leads to the introduction of propargyl groups onto RNA instead of native methyl (FIG. 1b)(10). Cu(I)-catalyzed azide-alkyne cycloaddition (CuAAC) reaction is then carried out directly on cultured cells to functionalise RNA with a click-degrader, which catalyses the cleavage of RNA, leading to its degradation (FIG. 1c). Without wishing to be bound by theory, the inventors believe that the imidazole-based click-degrader works through a dual mechanism. First, it acts as a general base, abstracting a proton from the 2′O position on RNA leading to cleavage, similarly to ribonucleases (FIG. 1d)(11). Secondly, it cleaves RNA in a copper-dependent manner, mimicking several nucleic acid-cleaving natural products (FIG. 1e)(12). As a result, this cleavage leads to a decrease of the methylated substrates which can be directly quantified, thus providing information about the methylation status of any RNA transcript in real-time.

Slick-Seq Degrades RNA Through a Dual Chemical Mechanism

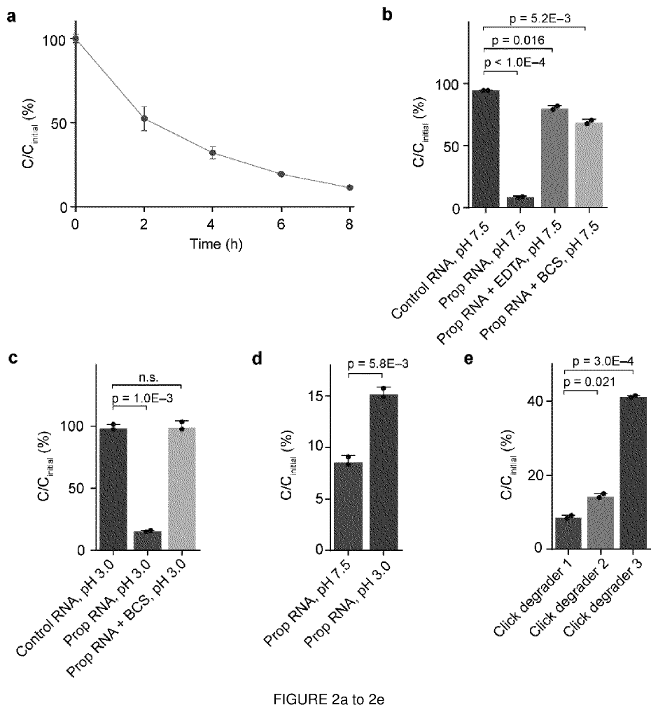

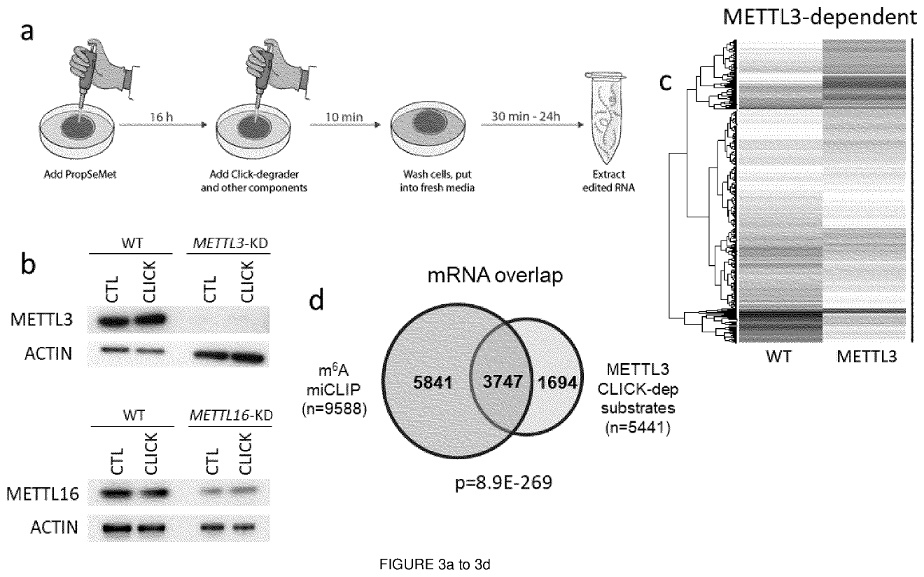

To demonstrate the validity of the RNA degradation strategy and to probe its mechanism, the inventors carried out in vitro experiments on RNA 11-er functionalized with a propargyl moiety on one of its ⁶A positions, while using a non-modified 11-mer as a control. The inventors went on to install the click-degrader on alkynylated RNA in order to observe what effect it has on RNA stability. Under optimal conditions, CuAAC RNA functionalization was complete in approximately 10 min (FIG. 5a-c). Furthermore, the inventors found that upon incubation at 37° C. the functionalized RNA gets gradually degraded (FIG. 2a). Although the kinetic profile of RNA degradation may be complex, first-order half-lives are often used as an estimate of RNA stability(13). Using a first-order approximation the inventors calculated the functionalized RNA 11-er to have a half-life of 2.6 hours (FIG. 5d). While this result was observed using a short RNA oligomer, the inventors anticipate longer RNAs to have more sites susceptible to degradation and should be degraded faster. Only 9% of the click-degrader functionalized RNA remained after 14 hours of incubation, whereas almost no degradation was observed when using a control RNA oligomer lacking the propargyl handle and the ability to be click-functionalized (FIG. 2b). This demonstrates that only the covalently imidazole-functionalized RNA is degraded thus the observed effect is specific to modified RNA and not a result of the individual components in a reaction.

To gain insight into the mechanism of click-degraders, the inventors carried out in vitro degradation reactions under conditions which suppress certain mechanisms. To investigate whether copper plays a role, the inventors ran reactions quenched either by a copper chelator bathocuproinedisulfonic acid (BCS) or a general metal chelator ethylenediaminetetraacetic acid (EDTA). With these reaction additives, 68% and 79% of RNA remained intact, respectively (FIG. 2b), suggesting that copper indeed plays a key role in the degradation mechanism. To investigate the role of click-degrader as a general base, the inventors carried out the reaction under acidic conditions (pH 3), in which imidazole of the click-degrader is protonated and non-basic while intrinsic RNA stability is comparable to neutral conditions(14). In this case, 15% of functionalized RNA were retained (almost twice as much as under pH 7.5), whereas the non-propargylated oligomer was not significantly affected (FIG. 2c,d). Importantly, no significant RNA degradation was observed when the reaction was carried out with a copper chelator bathocuproinedisulfonic acid (BCS) at pH 3, with both copper and general base mechanisms being blocked (FIG. 2c). These findings show that both copper-dependent and general base mechanisms are relevant and sufficient to explain the chemical mechanism of click-degraders. In addition, the inventors also investigated how the length of the click-degrader linker affects the efficiency of degradation. The inventors tested three different linker lengths (2, 4 and 6 PEG subunits) and found that the longest linker illustrated the most competent degradation, possibly due to a better reach (FIG. 2e). However, isolating click-degraders with longer linkers was challenging, thus the inventors used click-degrader 1 (6 PEG subunits) for downstream experiments.

Elucidation of mRNA Substrates of m⁶A Writers

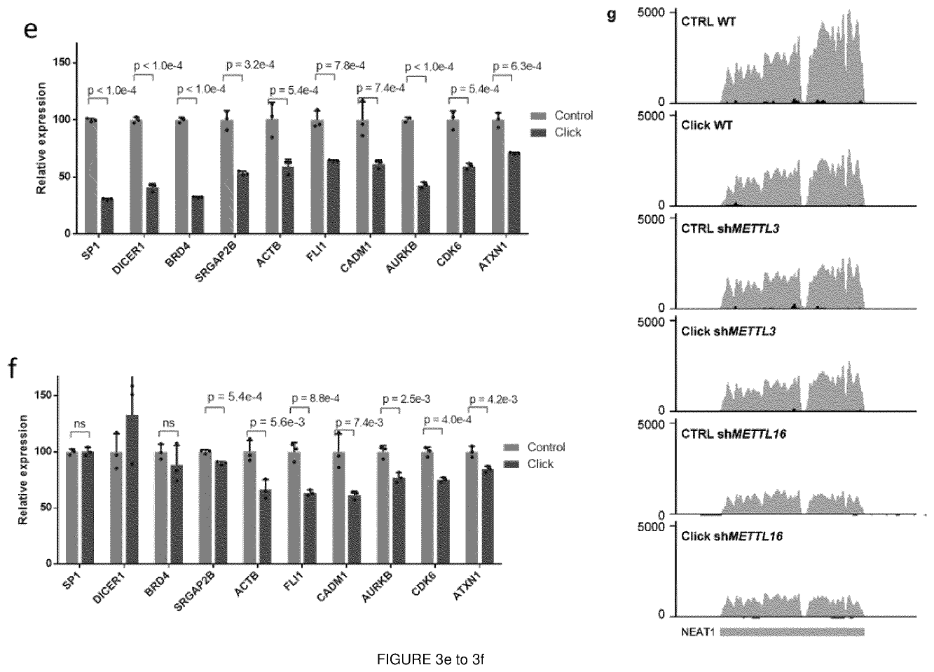

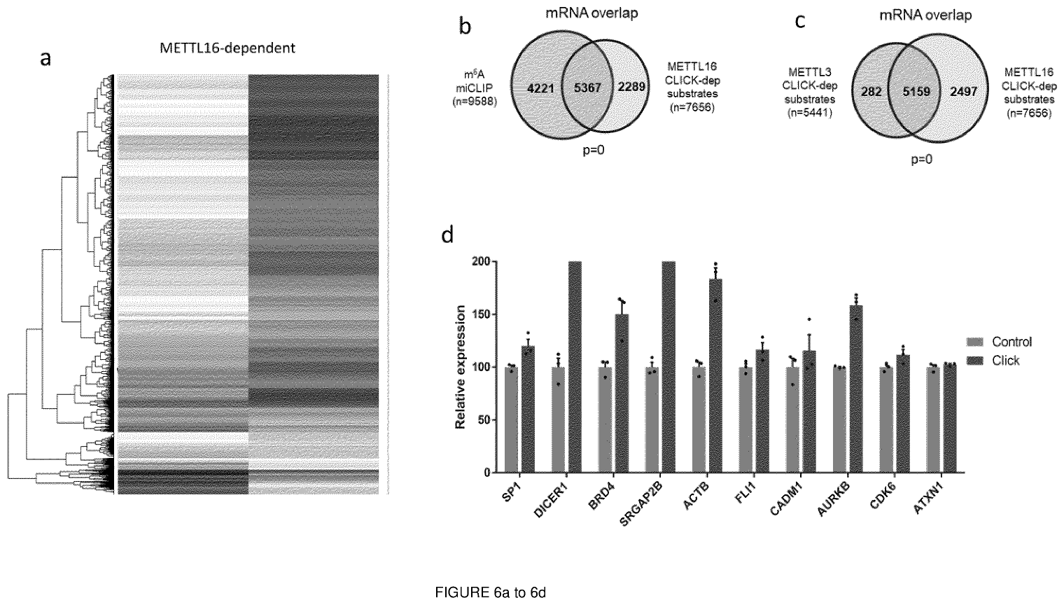

Upregulation of RNA methylation was implicated to play a pivotal role in the maintenance of cancer including acute myeloid leukaemia cells (AML)(15, 16). Therefore, to demonstrate that this platform can be used to elucidate the RNA methylation landscape in a cancer model, the inventors went on to apply Slick-Seq using MOLM-13, a human AML cell line (FIG. 3a). For this purpose the inventors used isogenic MOLM-13 cells stably transduced with conditional shRNAs against METTL3 and METTL16, both known to be m⁶A writers(17, 18), or scrambled shRNA as control (FIG. 3b)(19). As the platform is purely catalytic-dependent, the loss or down-regulation of an RNA methyltransferase will lead to the absence of the click-based RNA degradation of its RNA substrates. Initially, the inventors observed that the abundance of many mRNAs was reduced in clicked but not control cells, which suggests successful intracellular degradation of methylated transcripts (FIG. 3c, 6a). Strikingly, many of these transcripts remained unaffected in cells with either METTL3 or METT16 downregulation, suggesting that the modification on those substrates is a result of catalytic activity of the corresponding MTase. Using the present platform, the inventors identified 5411 METTL3- and 7656 METTL16-dependent mRNA substrates. The inventors cross-compared these findings to results of a reported m⁶A miCLIP sequencing experiment carried out on MOLM-13 cells (20). The inventors observed a consistent overlap for both enzymes—69% of METTL3 and 67% of METTL16 mRNA substrates identified through Slick-Seq were found to contain m⁶A sites via miCLIP (FIG. 3d, 6b). Furthermore, the inventors found that the m⁶A methylation of 5159 mRNAs depends on both MTases and the great majority of the identified methylated substrates are dependent on METTL16, which goes in line with the fact that METTL16 is the modulator of cellular cofactor SAM and its levels (FIG. 6c)(17, 21). To see whether these results can be observed via independent methods, the inventors used RT-qPCR to validate a panel of m⁶A-containing transcripts (FIGS. 3e, f, 6d). Notably, the validation mirrored the results of the RNA-seq highlighting the specificity of our platform. Thus, the inventors have demonstrated that Slick-Seq can specifically identify RNA methylase mRNA substrates and recapitulate the results of antibody-based approaches.

Many IncRNAs are METTL3 and/or METTL16 Substrates

Another group of RNAs reported to be heavily methylated is the long non-coding RNAs (IncRNAs). The inventors therefore investigated whether our Slick-Seq platform could efficiently identify methylation changes on that RNA subgroup focusing again on METTL3- and/or METTL16-dependent m⁶A(22). For example, NEAT1 was shown to be a target of m⁶A demethylase ALKBH5, although the MTases which deposit the modifications were not identified (23). Through Slick-Seq the inventors have demonstrated that the methylation of NEAT1 depends on both METTL3 and METTL16 (FIG. 3g). In total, the inventors identified 689 METTL3-dependent and 889 METTL16-dependent IncRNAs, out of which 77 and 104 were significantly overlapping with IncRNA substrates containing m6A sites via miCLIP sequencing (FIG. 6e, f). Interestingly, the inventors observed that the overlap between Slick-Seq and m⁶A miCLIP is much smaller for IncRNAs than for mRNAs, albeit significant. Furthermore, Slick-Seq was able to identify a greater number of IncRNA substrates than m⁶A miCLIP, the opposite to what was observed for mRNAs, suggesting that the present platform efficiently probes IncRNA methylation without antibody biases. Similar to mRNAs, majority of IncRNAs are promiscuous m⁶A substrates as methylation of 562 IncRNAs (56% of total identified) appeared to be dependent on both METTL3 and METTL16 (FIG. 6g). The inventors were also able to recapitulate a number of these findings via RT-qPCR (FIG. 6h). With Slick-Seq the inventors have thus revealed a previously unrecognized m⁶A methylation on IncRNAs in an MTase-dependent fashion.

Slick-Seq Reveals m⁶A being Widespread in Intronic and Intergenic Regions.

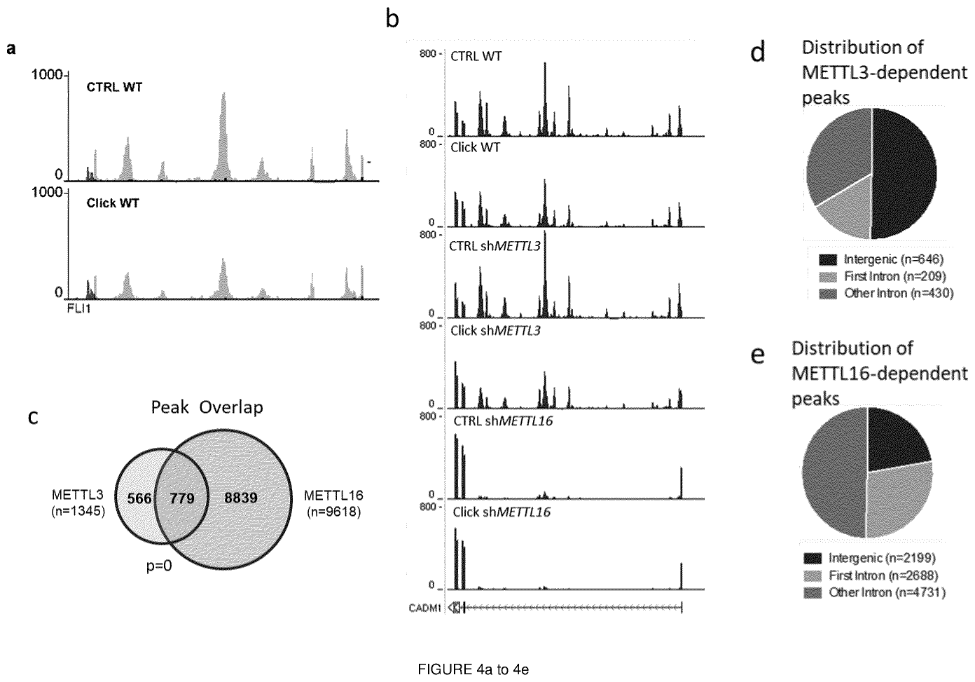

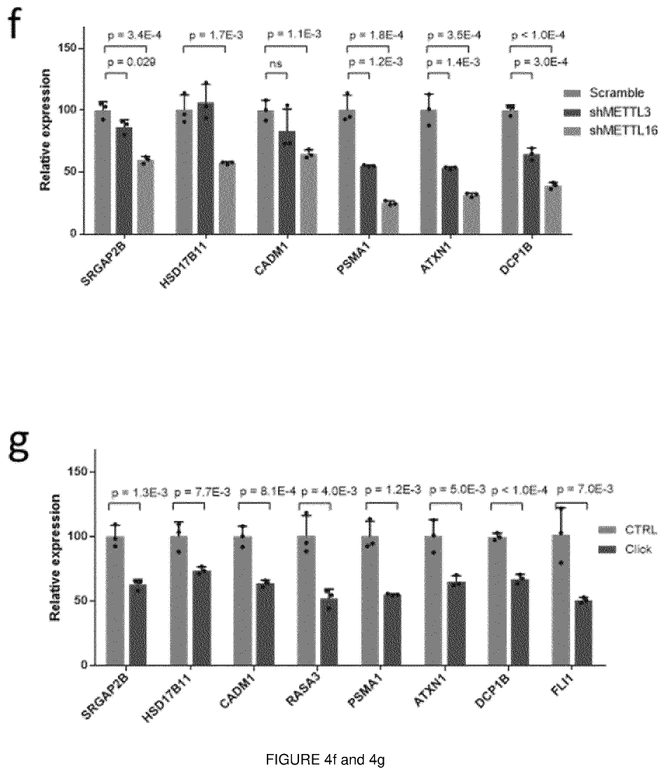

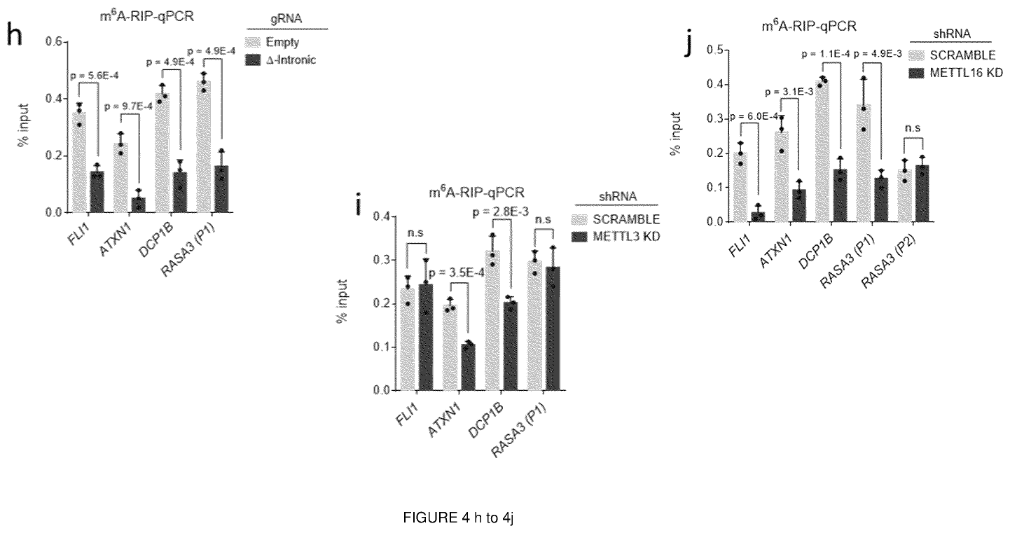

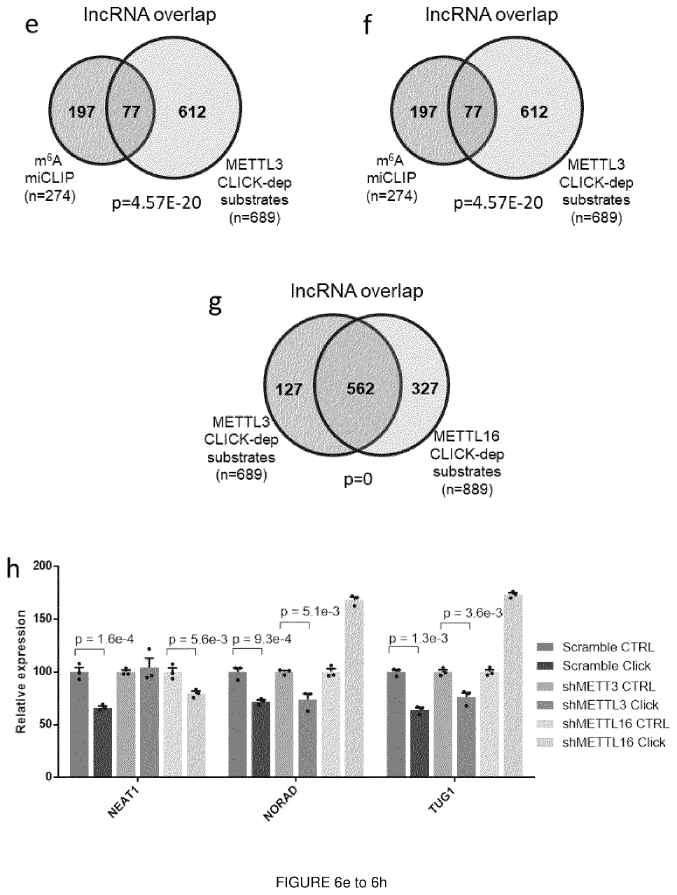

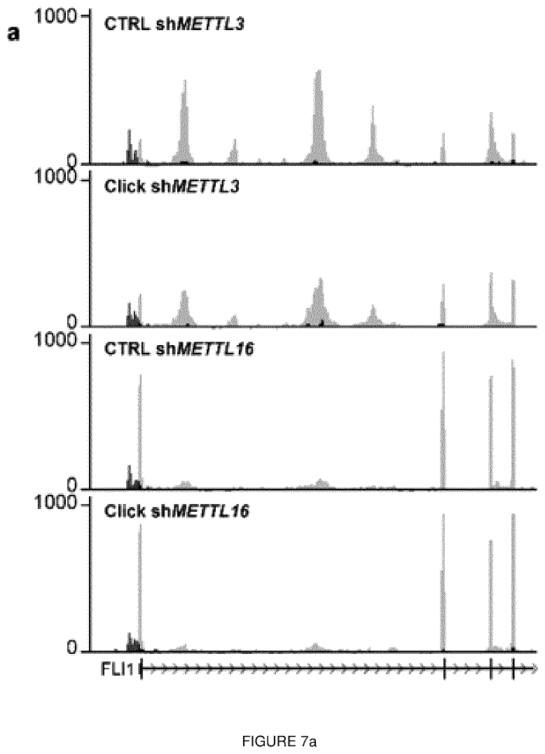

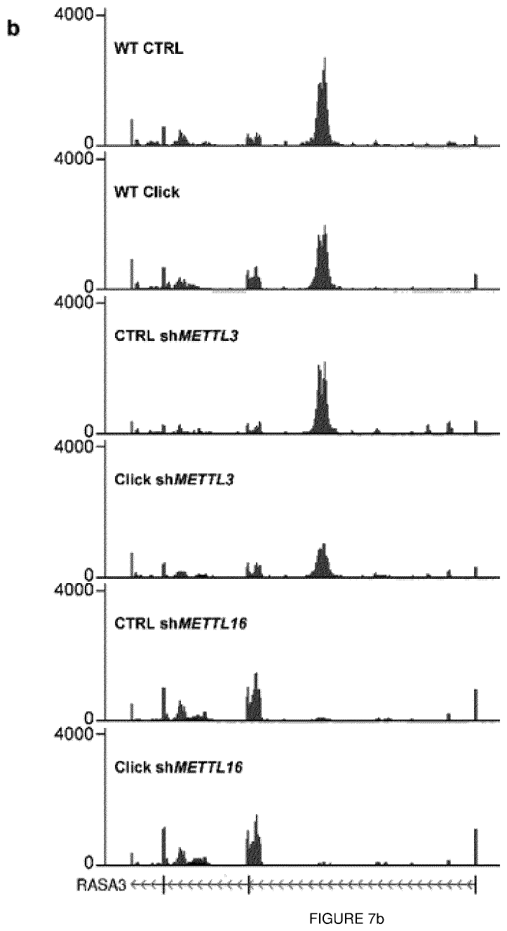

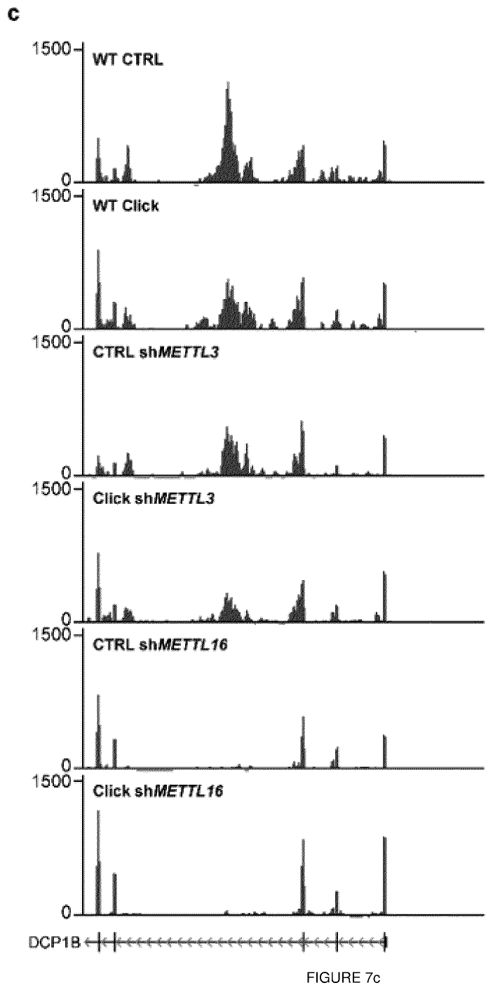

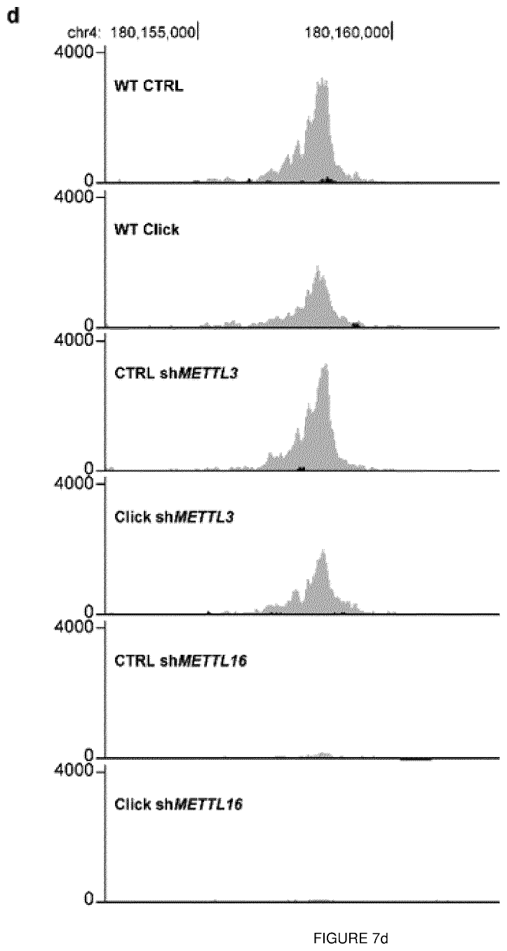

Rather unexpectedly, further interrogation of our RNAseq data using the Slick-Seq platform revealed peaks in intronic and intergenic regions. The majority of these peaks were highly sensitive to functionalization with the click-degrader, indicating that these regions were indeed modified by MTases (FIG. 4a). Another interesting observation was the pronounced loss of abundance of many identified peaks in cells depleted for either METTL3 or METTL16, implying that these MTases regulate the methylation in the corresponding peaks (FIG. 4b, FIG. 7a-d). In total the inventors have found 1345 METTL3- and 9618 METTL16-dependent intronic and intergenic peaks (FIG. 4c) with the distribution of these peaks appearing different for the two MTases. In line with the recent literature (15, 24, 25), 50% of METTL3-dependent peaks were found in intergenic regions (compared to 23% for METTL16) further highlighting the involvement of METTL3 in chromatin associated or co-transcriptional pathways (FIG. 4d, e). Moreover, more than 77% of METTL16-dependent peaks were belonging to intronic regions with 36% of those found in the first intron—unexpected for a 3′-biased method. This highlights the different roles these MTases play in mRNA modification, with METTL16 regulating the modification in many more intronic regions than METTL3. This goes in line with previous reports about the co-transcriptional role of METTL3 and METTL16 depositing m⁶A into introns (17, 26). Slick-Seq is thus the first method to discover a widespread methylation in low-abundance transcriptomic regions. Unlike antibody-based intronic analysis, Slick-Seq does not require enrichment of nascent RNA to observe intronic methylation.

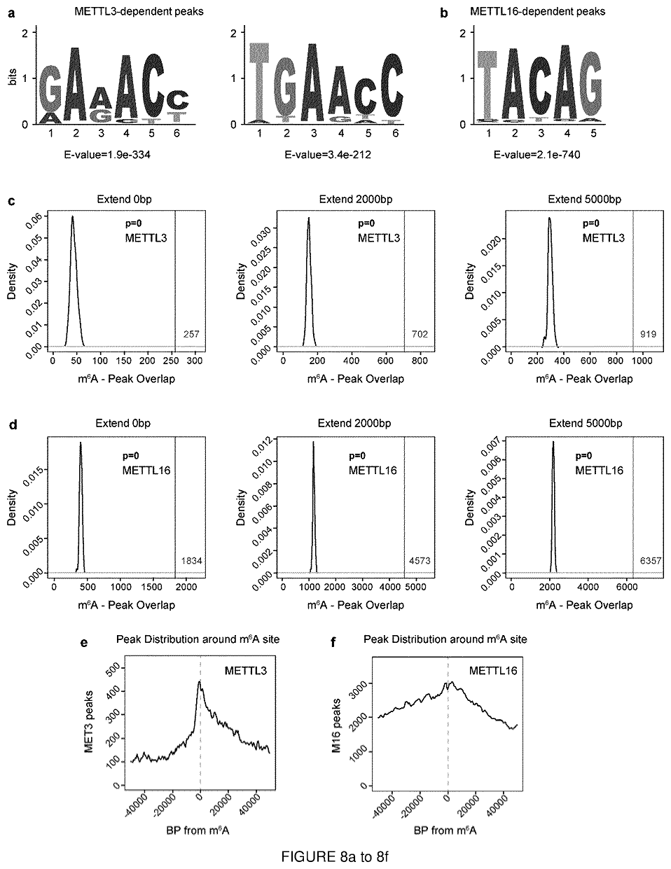

To further demonstrate that the peaks the inventors found contain bona fide m⁶A sites, the inventors performed motif analysis. The inventors have found variations of DRACH motif and TACAG, the reported consensus sequences for METTL3 and METTL1 6, dramatically overrepresented in METTL3- and METTL16-dependent peaks, respectively (FIG. 8a,b)(21, 27, 28). The inventors also compared the overlap between these peaks and m⁶A sites found in introns and exons through m⁶A miCLIP. A significant overlap was observed for both METTL3- and METTL16-dependent peaks, providing further evidence that the observed peaks indeed have m⁶A sites (FIG. 8c,d). Furthermore, a centred distribution of distances between miCLIP sites and Slick-Seq peaks was observed (FIG. 8e,f). The inventors have validated some of these peaks via RT-qPCR and found that their enzyme dependency mirrors the ones observed in the RNA-seq results (FIG. 4f). Furthermore, the inventors have validated a number of peaks being sensitive to the click reaction (FIG. 4g). To validate the intronic results with additional, independent methods, the inventors derived cell lines with deletions in intronic regions corresponding to METTL3- or METTL16-dependent Slick-Seq peaks. This was done using CRISPR-Cas9, with dual gRNAs targeting the flanks of selected Slick-Seq peaks. Having analysed the RNA contents via m⁶A-RIP, the inventors found that the cells with removed introns via CRISPR had significantly less m⁶A compared to cells containing empty CRISPR-Cas9 vectors (FIG. 4h). Additionally, in MOLM-13 cells with METTL3 or METTL16 knock-down, the m⁶A-RIP signal was selectively decreased for peaks dependent on each RNA-modifying enzyme further validating the results of Slick-Seq (FIG. 4i,j). Combined, this shows that the Slick-Seq peaks do indeed contain m⁶A sites and hold information about methylase specificity.

METTL16 is Linked to Intronic Polyadenylation Sites.

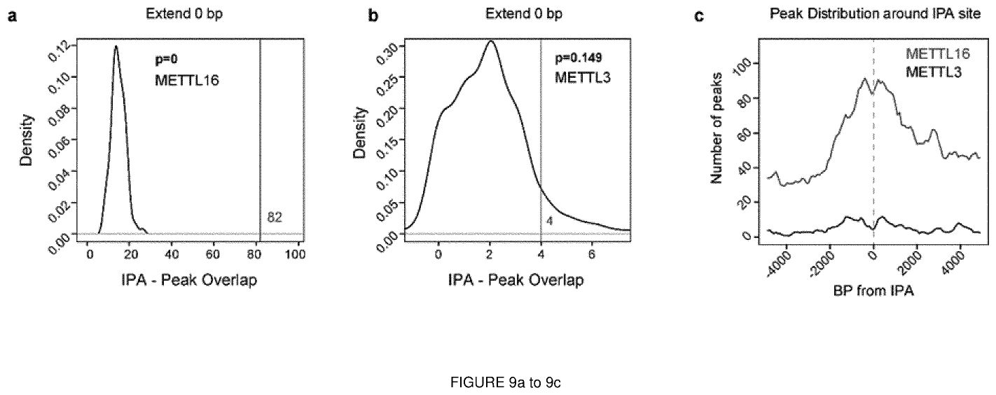

To better understand the presence of intronic peaks in our analysis, the inventors decided to investigate the connection to intronic polyadenylation (IPA) given the fact that polyA-enriched RNA was used for RNA-seq analysis. IPA has been recently reported to be widespread in cancers and inactivate tumour suppressors in leukaemia (29, 30). To determine whether IPA is associated with the presence of m⁶A, the inventors compared the positions of Slick-Seq peaks to IPA sites of leukemia cells identified in a published study using 3′-Seq(29). For additional confidence, the inventors applied very strict parameters and only considered Slick-Seq peaks positioned on the exact IPA site. Strikingly, a significant overlap was found between METTL16-dependent peaks and the IPA sites but not for METTL3 (FIG. 9a,b). Moreover, the distribution of METTL16 peaks around the IPA sites suggest a link between the two, with a large number of the peaks overlapping or being close to the IPA sites (FIG. 9c). These data not only suggest that the MTase METTL16 has a possible role in IPA and splicing but also demonstrate how Slick-Seq can be utilized to interrogate the function of methylation in low-abundance transcriptomic regions.

One Pot Mixed RNA

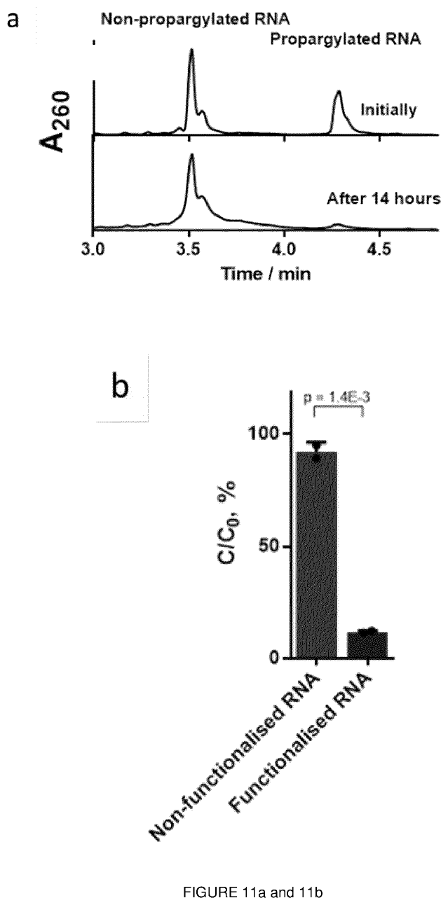

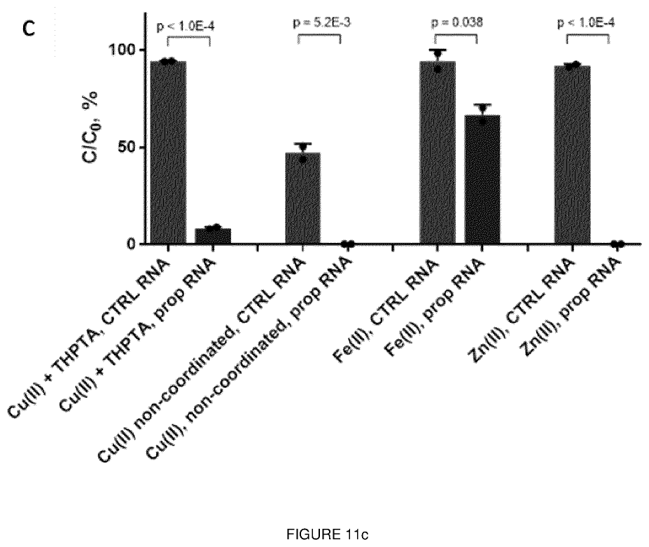

When degradation is carried out on a mixture of propargylated and non-propargylated RNA oligonucleotide, specific RNA degradation of the propargylated RNA was observed (FIGS. 11A and 11B).

Evaluation of Other Divalent Metal Ions

Free copper and zinc were found both result in a more complete degradation than copper-THPTA complex used for click chemistry (FIG. 11C). However, free Cu (II) also results in non-specific degradation of non-propargylated RNA. Iron (II) has minimal influence on the extent of RNA degradation—almost the same results is observed without any metal (67% vs 68% of RNA intact, respectively). n=2, error bars correspond to SD.

The inventors have developed Slick-Seq, a powerful small molecule-based method to elucidate multiple aspects of the RNA methylation landscape within cells, including high-confidence mapping of methylation in introns and intergenic regions. Unlike many RNA methylation sequencing methods, Slick-Seq is non-biased, it strictly depends on the catalytic activity of RNA methylases and can determine their substrate specificity. It offers the reproducibility characteristic to small molecule-based methods, is very easy to carry out and has low RNA input requirement, making it ideal for parallel characterization of different cell types, rare purified populations and tissues. Whereas antibody-based methods provide a biased picture of methylation at any given time, Slick-Seq reports a dynamic state of methylation in a cell.

The inventors have utilized Slick-Seq to determine the substrates of m⁶A writers METTL3 and METTL16 as well as their overlap. The inventors have also demonstrated that m⁶A is widespread in IncRNAs as well as intronic and intergenic regions, deposited mostly by METTL16. Furthermore, the inventors demonstrate for the first time the prevalence of m⁶A modification in polyadenylated introns, which have oncogenic contributions in cancer (29, 30).

Slick-Seq is highly modular and with suitable isogenic models it can be adapted for the study of other RNA methylases and demethylases, as well as other RNA modifications. The majority of studies on RNA methylation focus on high-abundance RNA species whereas the role of methylation in low abundance species is largely uncharacterized due to lack of molecular tools. As such, the inventors foresee Slick-Seq enabling the study of RNA modifications in previously inaccessible regions of the transcriptome. Defects in intron-related mechanisms are implicated in a wide range of pathologies(31) and much is yet to be learned about the role of non-coding RNAs in disease(32). The inventors thus expect Slick-Seq to facilitate both fundamental and translational discoveries about RNA modifications throughout the transcriptome.

Investigation of acCLICK-Seq RNA Degradation

The inventors have developed acCLICK-Seq, a small molecule-based transcriptome editing platform which exploit endogenous RNA acetylation pathways, such as the NAT10 enzymatic pathway in order to deposit an alkyne group, instead of the natural acetyl moiety, to the RNA at the N₄ position of cytidine. This alkyne group serves as the scaffold for the copper(I)-catalysed azide-alkyne cycloaddition reaction with a bifunctional probe (FIG. 13a). Enzymatic labelling is performed by culturing the cells in a medium containing ethyl-3-butynoate resulting in incorporation of the alkyne moiety into cellular RNA (FIG. 13b). The resulting cells are subsequently treated with a click degrader which comprises an azide group as a bioorthogonal handle for covalent linkage to the alkyne-functionalized RNA and an imidazole group, such as that described for Slick-Seq (FIG. 13c) (36).

Total click-degrader-treated RNA is then sequenced; observation of decreased signal due to the incorporation of the bifunctional probe relative to the ‘unclicked’ RNA provides a method for identifying N₄-acetylcytidine (ac4C) modified transcripts. Since acCLICK-Seq requires no non-standard RNA and lengthy in vitro processing procedures usually found in typical antibody- and small molecule-based methods, it is advantageous in preserving the authenticity of modification sites and minimizing detection bias. By avoiding a pulling-down enrichment method, this platform also potentially provides higher sensitivity to study the prevalence (or otherwise) of ac4C modifications in low-abundance RNA species such as in poly(A) transcripts across cell lines of relevance to blood cancer.

Elucidation of mRNA Substrates of Acetyltransferases

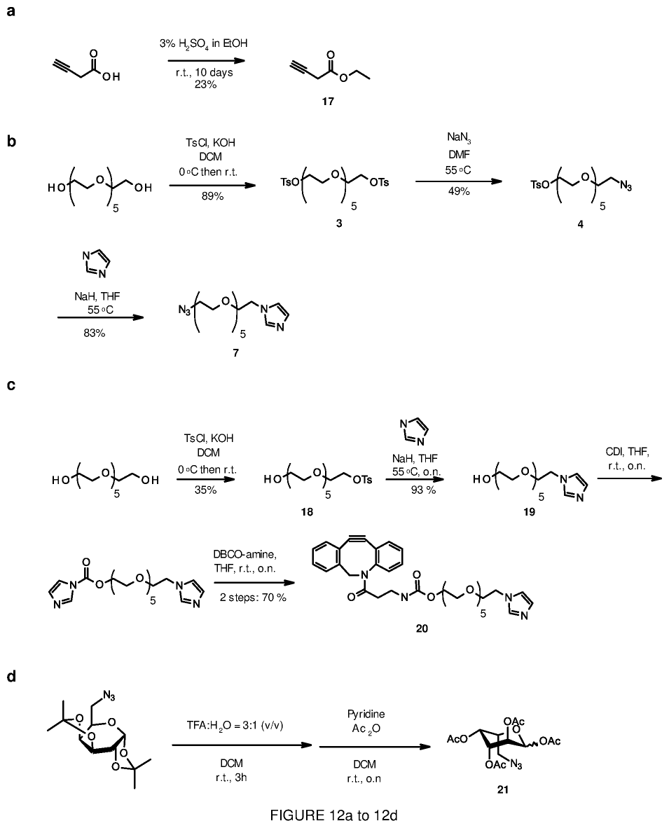

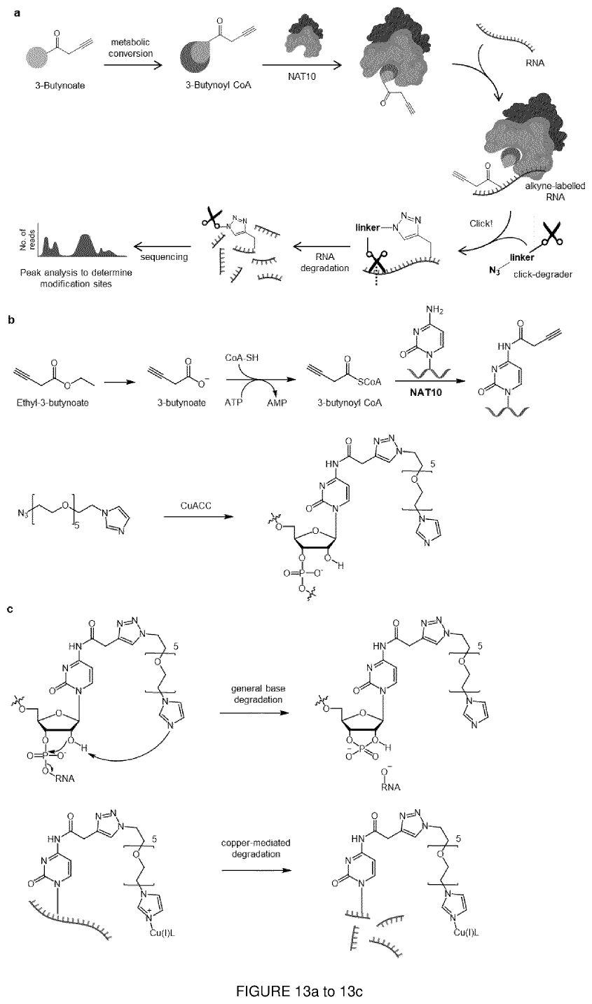

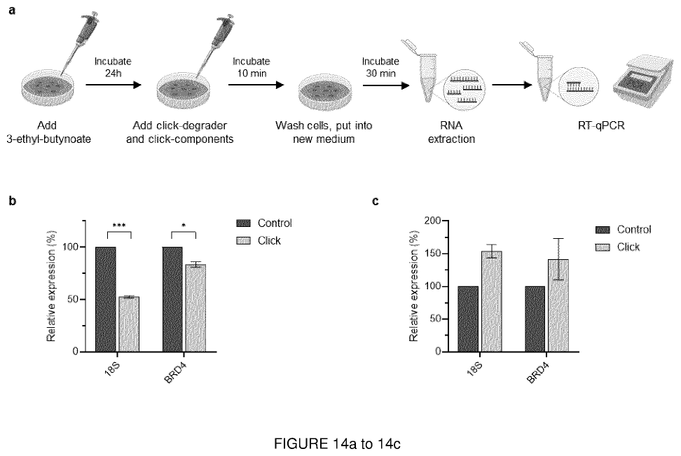

The cellular labelling probe, ethyl-3-buytnoate (FIG. 12a) and click-degrader, bearing an azide conjugated to an imidazole via PEG linker (FIG. 12b), were synthesized and then acCLICK-Seq was directly performed in cells. The first step of acCLICK-Seq involves culturing the cells (MOLM13) in a medium containing ethyl-3-butynoate for 24 hours, to allow enzymatic labelling of cellular RNA with the alkyne tags. The resulting cells were then treated with the azide bearing the click-degrader and other click components that promoted copper-catalysed click reaction followed by immediate RNA degradation (FIG. 14a). In this way, ac4C sites are marked and the degradation signals can be observed via qPCR measurement (FIG. 14b). To demonstrate the adeptness of the method the inventors initially looked at 18S and BRD4 gene as the qPCR targets. The 18S transcript was specifically chosen as it is the most abundant and well-reported ac4C sites whereas BRD4 gene is one of the reported low-abundance mRNA transcripts (37, 38).

A reduction was observed at the relative expression levels of the two target transcripts in the clicked but not control (ethyl-3-butynoate-fed, but not clicked) cells, which indicates successful intracellular degradation of ac4C modified transcripts. Furthermore, the expression levels of both 18S and BRD4 transcripts were rescued in cells with NAT10 knocked-out (FIG. 14c), implying that the acetylation of these RNAs are mediated by the enzymatic activity of NAT10 and that the acCLICK-Seq platform could site specifically tag and degrade the relevant NAT10-dependent ac4C modifications. These preliminary results recognize of the competency of the acCLICK-Seq for a comprehensive identification and study of ac4C modification.

Investigation of glycoCLICK-Seq RNA Degradation

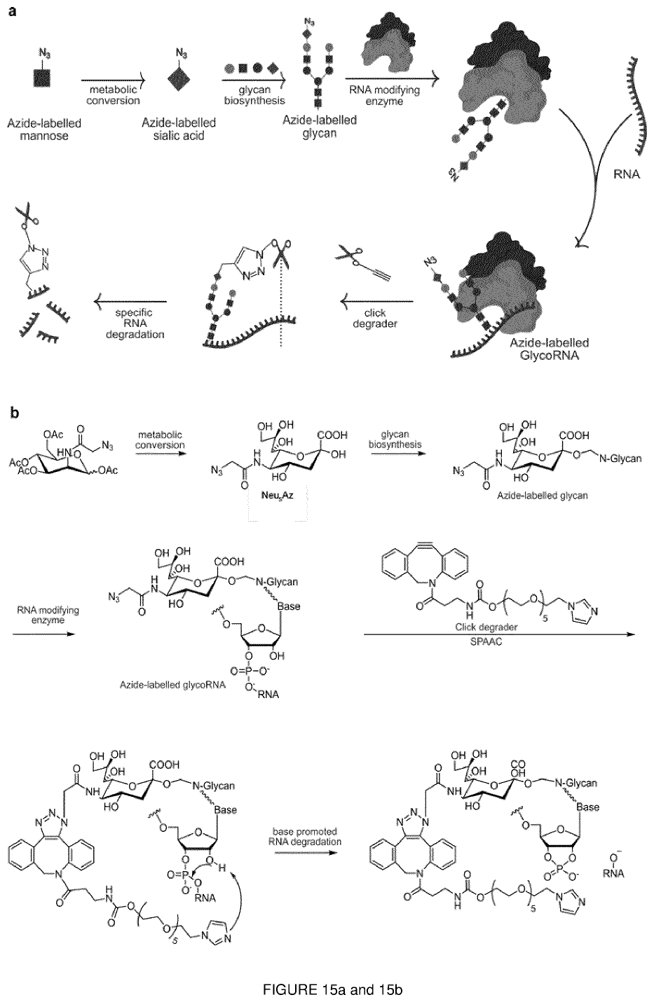

The inventors have developed glycoCLICK-Seq, a small molecule-based transcriptome editing platform which can be used to map RNA glycosylation sites. In glycoCLICK-Seq, click-degraders are used in tandem with a glycosylation probe, which may be an unnatural azide-containing analoge of mannose, Ac₄ManNAz (N-azidoacetylmannosamine-tetraacylated) (39) (FIG. 15). In a typical glycoCLICK-Seq procedure, cells are cultured in a medium containing a monosaccharide analogue, such as Ac₄ManNAz, to incorporate an azide label into cellular RNA. The resulting cells are then treated with a ‘click degrader’ which comprises a bioorthogonal handle for covalent linkage to the azide-functionalized RNA and an imidazole group that is capable of degrading RNA in a similar mechanism to that of ribonucleases. Total click-degrader-treated RNA is then sequenced; observation of decreased signal due to the incorporation of the ‘click degrader’ relative to the ‘unclicked’ RNA provides a method for identifying glycosylated RNA. The method can be used to study the prevalence (or otherwise) of glycoRNA across cell lines of relevance to blood cancer. Unlike antibody- and small molecule-based methods, glycoCLICK-Seq requires no non-standard RNA processing procedures hence preserves the nativity of glycoRNA species and has minimum detection bias (36). By avoiding the pulling-down enrichment method, this platform also provide potentially higher sensitivity to map glycosylation in low abundance RNA species.

glycoCLICK-Seq Degrades RNA

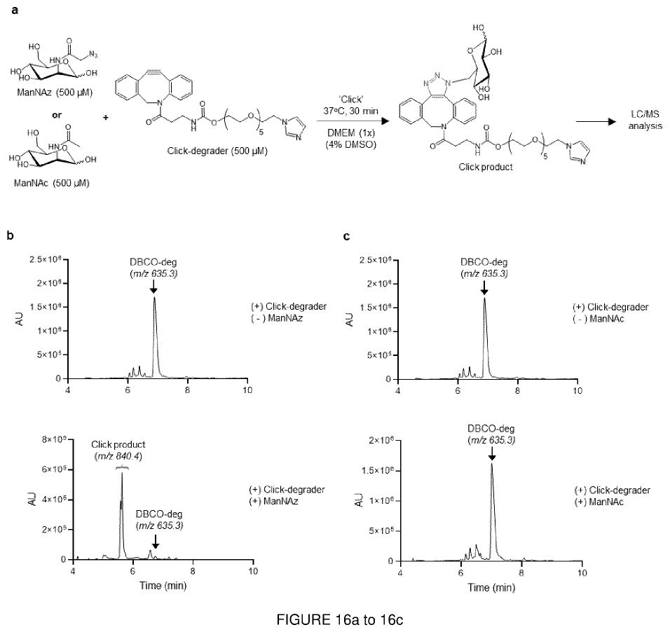

The inventors synthesized a click-degrader bearing dibenzyl-cyclooctyne (DBCO) conjugated to an imidazole via a PEG linker (FIG. 12c). An in-vitro click reaction was then performed with the glycosylation probe, ManNAz. The reaction was performed under cell-culture media at 37° C. to mimic the cell physiological conditions and analysed by HPLC-LCMS measurement. Complete depletion of the click-degrader was observed, along with formation of the new click-product (Ac₄ManNAz-degrader) after 30 min (FIGS. 16a and b). There was no formation of the click-product observed when non-azide sugar probe ManNAc was used as a control (FIG. 16c).

Elucidation of mRNA Substrates of Glycosylation Enzyme

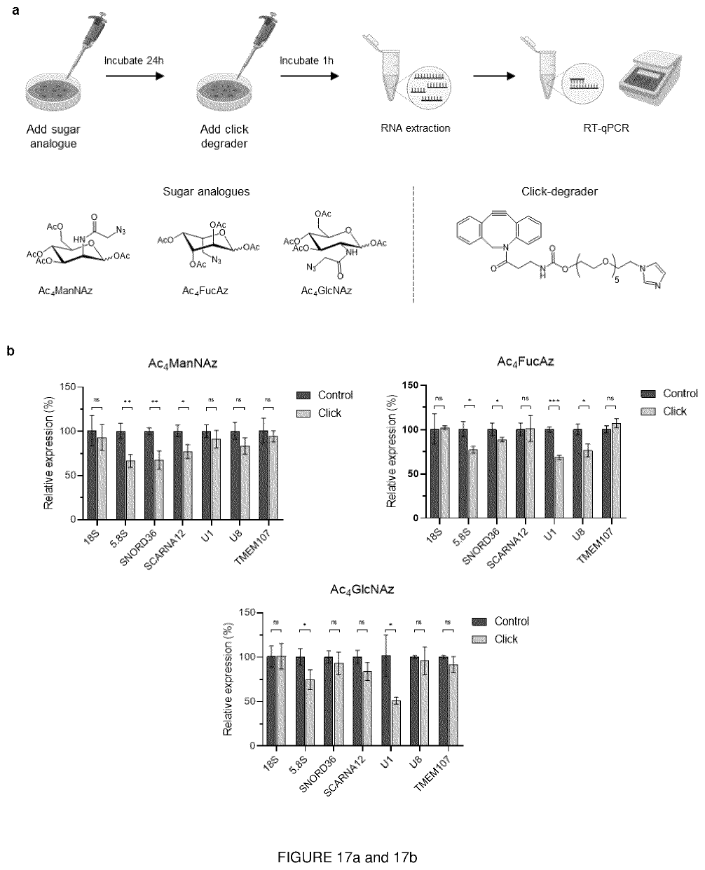

The workflow of gycoCLICK-Seq requires culturing the cells (HeLa) in a medium containing Ac₄ManNAz for 24 hours, for enzymatically incorporating the azide label into cellular RNA. The resulting cells are treated with the click-degrader that promotes immediate RNA degradation (FIG. 17a). In this way, glycoRNA sites are marked and the degradation signals can be observed via qPCR and high-throughput sequencing. In addition to Ac₄ManNAz, two other azide sugar probes, Ac₄FucAz and Ac₄GlcNAz, were used to demonstrate the range of functional sugar analogues and effectivity of glycoRNA probing by different sugar analogues (FIGS. 12d and 17a).

Analysis from the qPCR results shows a decrease in RNA expression levels across the panel of reported glycoRNA transcripts (40) (5.8S, SNORD36, SCARNA12, U1, and U8) for all three of the sugar probes but not on the control RNA (18S and TMEM107) (FIG. 17b). All the non-control transcripts were chosen for the reasonable abundant detection of glycoRNA, and the feasibility to design qPCR primers. Among all the transcripts observed, the decrement of 5.8S expression relative to the control is prominent and agreeable across all of the three sugar probes used, indicating the possibility of 5.8S as an eminent glycoRNA transcript. These results established an insight of the potential and specificity of glycoCLICK-Seq to unfold the existence of glycoRNA independently to the first reported finding, in a straightforward and reliable manner.

Tables

TABLE 1
List of detected RNA species.

| | | Calcd. | Observed |
|---|---|---|---|
| Label | Species | mass | mass |
| i | Propargylated RNA 11-mer | 3559 | 3560 |
| ii | Non-propargylated RNA 11-mer | 3521 | 3522 |
| iii | Click-degrader 1 functionalised RNA 11-mer | 3916 | 3917 |
| iv | Click-degrader 2 functionalised RNA 11-mer | 3828 | 3830 |
| v | Click-degrader 3 functionalised RNA 11-mer | 3740 | 3742 |

TABLE 2
List of primers.

| | | |
|---|---|---|
| Name | Forward primer sequence (5′-3′) | Reverse primer sequence (5′-3′) |

Primers for mRNA exon analysis (FIG. 3e, 3f, 6d)

| | | |
|---|---|---|
| SP1 | CAGTGGGCTACAGGGGTCT | CTTGCAATGAGCCTCCAGAT |
| DICER1 | CTTAAAGTTGTTAGTGAGTGGAATGAA | CTGTTATCTATCCTGTTATCAACCAAA |
| BRD4 | GACATGAGCACAATCAAGTC | GAACACATCCTGGAGCTTGC |
| SRGAP2B | CCCTCGAGAGAAGCGGTCTT | GGTCCAGGCATTTCATCTGC |
| ACTB | CCAACCGCGAGAAGATGA | CCAGAGGCGTACAGGGATAG |
| FLI1 | AACGTGCACAGGGGAGTGAGGG | GTTACAGCCTGACCTCGCAGCC |
| CADM1 | CTGCTGTTGCTCTTCTCCGCCG | TGGTCGCAACCTCTCCCTCGAT |
| AURKB | GGGAACCCACCCTTTGAGAG | GGGGTTATGCCTGAGCAGTT |
| CDK6 | CACACCGAGTAGTGCATCGCGA | GCAAGGCCGAAGTCAGCGAGTT |
| ATXN1 | AGAAAGAACACGCCTGAGCC | GTACAATCCGCCAACAGCAG |

Primers for IncRNA analysis (FIG. 6h)

| | | |
|---|---|---|
| NEAT1 | GCCTTCTTGTGCGTTTCTCG | TCCCAGCGTTTAGCACAACA |
| NORAD | GTGACCACTCTGTCGCCATT | AGAATGAAGACCAACCGCCC |
| TUG1 | ACGACTGAGCAAGCACTACC | CTCAGCAATCAGGAGGCACA |

Primers for intron analysis (FIG. 4f and 4g)

| | | |
|---|---|---|
| SRGAP2B | CCACACCCAGCCAAGCCAACTC | AGGAGGGAAGGTACAGGATGGAGT |
| HSD17B11 | TGCGCTGCACCCACTAATGTGT | ACACTCTGGGGACTGTGGTGGG |
| CADM1 | CCGGAGACAGTGGCATGGAGGA | AGGAGCTCGGCTTGGGAAACCT |
| RASA3 | GCCTGGATGACCTTCCCGTTGC | CCCAGTACCAGAGCACCCCGAT |
| PSMA1 | AGTCAGGCAAGCCTTCTGGAGA | ATGGGGCCTTTGTTCCAGCTGC |
| ATXN1 | GGCAACCCTCACCTCAACCTGT | TGATGCACGTGACCGGGAAGGA |
| DCP1B | TCAGTCTGGCCAGGTGTTTACCA | TCCAAAGCAAACAGAAGGAAAGAA |
| FLI1 | CCATGCCCTCTGCTTCCTGTGC | ATCTGCAGCCAGGCAACCTGTG |

Housekeepers for normalisation

| | | |
|---|---|---|
| 18S | GTAACCCGTTGAACCCCATT | CCATCCAATCGGTAGTAGCG |
| RPL32 | GTTCCTGGTCCACAACGTCA | CATTGTGAGCGATCTCGGCA |

Primers for rRNA and mRNA analysis (FIG. 12)

| | | |
|---|---|---|
| 18S | GTAACCCGTTGAACCCCATT | CCATCCAATCGGTAGTAGCG |
| BRD4 | CCTGGTGAAGAATGTGATGG | GTTGGTGCTGGCTGCGTTGG |

Housekeepers for normalization

| | | |
|---|---|---|
| 5S | AACGCGCCCGATCTCGTCTGAT | ATTCCCAGGCGGTCTCCCATCC |

Primers for rRNA and mRNA analysis (FIG. 14)

| | | |
|---|---|---|
| 18S | GTAACCCGTTGAACCCCATT | CCATCCAATCGGTAGTAGCG |
| 5.8S | GTGCGTCGATGAAGAACGC | AGTGCGTTCGAAGTGTCGAT |
| SNORD36 | GCAATGATGTGAATCTCTCACTGAA | TTGCTCAGGGTTAAAAAGCTCA |
| SCARNA12 | CATTTCTGGTGCTGCCCCTA | AGATCCAAGGTTGCGCTCAG |
| U1 | CTTACCTGGCAGGGGAGATAC | ACGCAGTCCCCCACTACCACAA |
| U8 | GTGGGATAATCCTTACCTGT | ACAGGAGCAATCAGGGTGTT |
| TMEM107 | GCATCTGGTGGTCGTCATCA | CCTGCTTGTCATACTCCTCGG |

Houskeepers for normalization

| | | |
|---|---|---|
| GAPDH | TCCACTGGCGTCTTCACC | GGCAGAGATGATGACCCTTTT |

TABLE 3
List of gRNAs.

| | | |
|---|---|---|
| Name | Forward primer sequence (5′-3′) | Reverse primer sequence (5′-3′) |

gRNAs used for generation of Δintronic cells (FIG. 4h-j)

| | | |
|---|---|---|
| DCP1B | GACAACTCACAATCACCTC | AGAGGTGATTGTGAGTTGTC |
| ATXN1 | GCTTTATCTGGTACTCATAG | CTATGAGTACCAGATAAAGC |
| RASA3(1) | GAAGCTGTCCGTGTTCACAT | ATGTGAACACGGACAGCTTC |
| RASA3(2) | GTTGCACAACTCGGTGAACG | CGTTCACCGAGTTGTGCAAC |
| FLI1 | GAAAGGTGAGCGTAAACGTG | CACGTTTACGCTCACCTTTC |

### REFERENCES

1. S. Nachtergaele, C. He, The emerging biology of RNA post-transcriptional modifications. RNA Biol. 14, 156-163 (2017).
2. M. Frye, S. R. Jaffrey, T. Pan, G. Rechavi, T. Suzuki, RNA modifications: what have we learned and where are we headed? Nat. Rev. Genet. 17, 365-372 (2016).
3. I. A. Roundtree, M. E. Evans, T. Pan, C. He, Dynamic RNA Modifications in Gene Expression Regulation. Cell 169, 1187-1200 (2017).
4. H. Shi, J. Wei, C. He, Where, When, and How: Context-Dependent Functions of RNA Methylation Writers, Readers, and Erasers. Mol. Cell 74, 640-650 (2019).
5. W. A. Cantara et al., The RNA modification database, RNAMDB: 2011 update. Nucleic Acids Res. 39, D195-D201 (2010).
6. K. D. Meyer et al., Comprehensive analysis of mRNA methylation reveals enrichment in 3′ UTRs and near stop codons. Cell 149, 1635-1646 (2012).
7. D. Dominissini et al., Topology of the human and mouse m6A RNA methylomes revealed by m6A-seq. Nature 485, 201-206 (2012).
8. B. Linder et al., Single-nucleotide-resolution mapping of m6A and m6Am throughout the transcriptome. Nat. Methods 12, 767-772 (2015).
9. M. Tomkuviene, B. Clouet-d'Orval, I. Cerniauskas, E. Weinhold, S. Klimasauskas, Programmable sequence-specific click-labeling of RNA using archaeal box C/D RNP methyltransferases. Nucleic Acids Res. 40, 6765-6773 (2012).
10. K. Hartstock et al., Enzymatic or In Vivo Installation of Propargyl Groups in Combination with Click Chemistry for the Enrichment and Detection of Methyltransferase Target Sites in RNA. Angew. Chem. Int. Ed. 57, 6342-6346 (2018).
11. R. T. Raines, Ribonuclease A. Chem. Rev. 98, 1045-1066 (1998).
12. Z.-R. Li et al., Macrocyclic colibactin induces DNA double-strand breaks via copper-mediated oxidative cleavage. Nat. Chem. 11, 880-889 (2019).
13. J. R. Peterson et al., Genome-wide gene expression and RNA half-life measurements allow predictions of regulation and metabolic behavior in Methanosarcina acetivorans. BMC Genomics 17, 924-924 (2016).
14. M. Oivanen, S. Kuusela, H. Lönnberg, Kinetics and Mechanisms for the Cleavage and Isomerization of the Phosphodiester Bonds of RNA by Brønsted Acids and Bases. Chem. Rev. 98, 961-990 (1998).
15. I. Barbieri et al., Promoter-bound METTL3 maintains myeloid leukaemia by m(6)A-dependent translation control. Nature 552, 126-131 (2017).
16. L. P. Vu et al., The N(6)-methyladenosine (m(6)A)-forming enzyme METTL3 controls myeloid differentiation of normal hematopoietic and leukemia cells. Nat. Med. 23, 1369-1376 (2017).
17. K. E. Pendleton et al., The U6 snRNA m(6)A Methyltransferase METTL16 Regulates SAM Synthetase Intron Retention. Cell 169, 824-835.e814 (2017).
18. J. A. Bokar, M. E. Shambaugh, D. Polayes, A. G. Matera, F. M. Rottman, Purification and cDNA cloning of the AdoMet-binding subunit of the human mRNA (N₆-adenosine)-methyltransferase. RNA 3, 1233-1247 (1997).
19. D. Wiederschain et al., Single-vector inducible lentiviral RNAi system for oncology target validation. Cell Cycle 8, 498-504 (2009).
20. A. Leger et al., RNA modifications detection by comparative Nanopore direct RNA sequencing. bioRxiv, 843136 (2019).
21. K. A. Doxtader et al., Structural Basis for Regulation of METTL16, an S-Adenosylmethionine Homeostasis Factor. Mol. Cell 71, 1001-1011.e1004 (2018).
22. D. Yang et al., N₆-Methyladenosine modification of lincRNA 1281 is critically required for mESC differentiation potential. Nucleic Acids Res. 46, 3906-3920 (2018).
23. J. Zhang et al., ALKBH5 promotes invasion and metastasis of gastric cancer by decreasing methylation of the IncRNA NEAT1. J. Physiol. Biochem. 75, 379-389 (2019).
24. H. Huang et al., Histone H3 trimethylation at lysine 36 guides m(6)A RNA modification co-transcriptionally. Nature 567, 414-419 (2019).
25. J. Liu et al., N (6)-methyladenosine of chromosome-associated regulatory RNA regulates chromatin state and transcription. Science 367, 580-586 (2020).
26. A. Louloupi, E. Ntini, T. Conrad, U. A. V. Ørom, Transient N-6-Methyladenosine Transcriptome Sequencing Reveals a Regulatory Role of m6A in Splicing Efficiency. Cell Rep. 23, 3429-3437 (2018).
27. M. Mendel et al., Methylation of Structured RNA by the m(6)A Writer METTL16 Is Essential for Mouse Embryonic Development. Mol. Cell 71, 986-1000.e1011 (2018).
28. H. Shima et al., S-Adenosylmethionine Synthesis Is Regulated by Selective N(6)-Adenosine Methylation and mRNA Degradation Involving METTL16 and YTHDC1. Cell Rep. 21, 3354-3363 (2017).
29. S.-H. Lee et al., Widespread intronic polyadenylation inactivates tumour suppressor genes in leukaemia. Nature 561, 127-131 (2018).
30. I. Singh et al., Widespread intronic polyadenylation diversifies immune cell transcriptomes. Nat. Commun. 9,1716 (2018).
31. H. Jung et al., Intron retention is a widespread mechanism of tumor-suppressor inactivation. Nat. Genet. 47, 1242-1248 (2015).
32. F. Kopp, J. T. Mendell, Functional Classification and Experimental Dissection of Long Noncoding RNAs. Cell 172, 393-407 (2018).
33. Freitas, A. A., Comprehensible classification models: a position paper. SIGKDD Explor. Newsl. 2014, 15(1), 1-10.
34. Tzelepis, K., CRISPR Dropout Screen Identifies Genetic Vulnerabilities and Therapeutic Targets in Acute Myeloid Leukemia. Cell Rep. 2016, 17 (4), 1193-1205.
35. Srivastava, G., Kaur, K. J., Hindsgaul, O. & Palcic, M. M. Enzymatic transfer of a preassembled trisaccharide antigen to cell surfaces using a fucosyltransferase. J. Biol. Chem. 267, 22356-22361 (1992).
36. Mikutis, S. et al. meCLICK-Seq, a Substrate-Hijacking and RNA Degradation Strategy for the Study of RNA Methylation. ACS Cent. Sci. 6, 2196-2208 (2020).
37. Sharma, S. et al. Yeast Kre33 and human NAT10 are conserved 18S rRNA cytosine acetyltransferases that modify tRNAs assisted by the adaptor Tan1/THUMPD1. Nucleic Acids Res. 43, 2242-2258 (2015).
38. Arango, D. et al. Acetylation of Cytidine in mRNA Promotes Translation Efficiency. Cell 175, 1872-1886.e24 (2018).
39. Saxon, E. Cell Surface Engineering by a Modified Staudinger Reaction. Science 287, 2007-2010 (2000).
40. Flynn, R. A. et al. Small RNAs are modified with N-glycans and displayed on the surface of living cells. Cell 184, 3109-3124.e22 (2021).

1. A method for cleaving a target nucleic acid molecule, the method comprising:
contacting the target nucleic acid with a bifunctional probe having the formula:
C-L-B
where C is a cleavage moiety, L is a linker and B is a binding moiety; such that the bifunctional probe covalently binds to the target nucleic acid molecule; and,
allowing the bifunctional probe to cleave the target nucleic acid molecule bound thereto.

2. The method of claim 1, wherein:
(i) the cleavage moiety is selected from substituted or unsubstituted imidazole (1,3-diazole), triazole, benzimidazole and azaindole; or
(ii) the cleavage moiety is selected from the groups represented by formula (I) to (III): 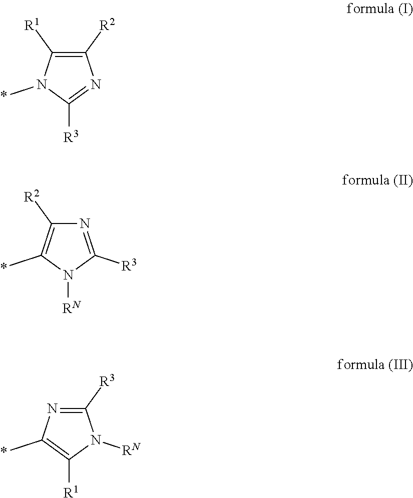
where: R¹, R² and R³ each independently represent a hydrogen atom or a C₁₋₆ alkyl group,
R^{N} represents a hydrogen atom or a C₁₋₆ alkyl group, and
represents the attachment position with the remainder of the probe (typically the linker unit L).

3.-4. (canceled)

5. The method of claim 1, wherein the linker comprises a polyalkylene glycol group.

6. The method of claim 1, wherein the target nucleic acid molecule comprises a partner moiety (P) to facilitate covalent binding of the bifunctional probe, preferably wherein the partner moiety is or comprises a group selected from alkynyl, alkenyl, isocyanaide (—N⁺≡C⁻).

7. The method of claim 6, wherein the binding moiety (B) is a covalent binding moiety (B_{C}) comprising a reactive group that is capable of forming a covalent bond with a partner moiety (P),
preferably wherein the covalent binding moiety is or comprises a group selected from azido (—N₃), nitrone (R′C═N⁺R″O⁻, where R″ is not H), nitrile oxide (—C≡N⁺—O⁻) or tetrazine.

8.-9. (canceled)

10. The method of claim 7, wherein the covalent binding moiety is or comprises an alkynyl (—C≡C—) group, and optionally wherein the partner moiety comprises an azido group.

11.-14. (canceled)

15. The method of claim 1, wherein the target nucleic acid molecule is an RNA molecule, and/or
wherein the target nucleic acid molecule is contacted with the bifunctional probe within a cell.

16. (canceled)

17. The method of claim 1, wherein the method is for selectively cleaving a target nucleic acid in a cell, wherein the method comprises:
contacting in the cell the target nucleic acid molecule, a nucleic acid modification enzyme and a cofactor analogue comprising a partner moiety, such that the nucleic acid modification enzyme tags the target nucleic acid with the partner moiety;
introducing into the cell the bifunctional probe, wherein the bifunctional probe has the formula (1):
C-L-B_{C} (1)
where C is a cleavage moiety, L is a linker and B_{C} is a binding moiety comprising a reactive group that reacts with the partner moiety to covalently bind the bifunctional probe to the target nucleic acid molecule.

18.-24. (canceled)

25. The method of claim 17, wherein:
(the nucleic acid modification enzyme is selected from a nucleic acid methyltransferase, a nucleic acid acetyltransferase, and a nucleic acid glycosyltransferase; and/or
(ii) the cofactor analogue is selected from an S-adenosylmethionine analogue, an acetyl-coenzyme A analogue, and a monosaccharide analogue or a glycan comprising the monosaccharide analogue.

26.-29. (canceled)

30. The method of claim 17, wherein the cofactor analogue is generated in the cell from an exogenous cofactor analogue precursor.

31.-34. (canceled)

35. A method for determining the modification of nucleic acid molecules by a nucleic acid modification enzyme in a cell comprising:
providing a first cell and a second cell, wherein the second cell has reduced or abolished expression or activity of the nucleic acid modification enzyme relative to the first cell, introducing in the first and second cells a co-factor analogue precursor comprising a partner moiety,
such that the co-factor analogue precursor is converted in the first and second cells into a co-factor analogue,
said co-factor analogue being a co-factor for the nucleic acid modification enzyme, such that nucleic molecules in the cell that contain a site of modification are tagged with the partner moiety in the presence of the nucleic acid modification enzyme,
introducing into the cells a bifunctional probe having the formula:
C-L-B_{C}
where C is a cleavage moiety, L is a linker and B_{C} is a binding moiety comprising a reactive group that reacts with the partner moiety to covalently bind the bifunctional probe to nucleic acid molecules tagged with the partner moiety,
allowing the bifunctional probe to cleave nucleic acid molecules in the first and seconds cells covalently bound to the bifunctional probe, and
identifying nucleic acid molecules which are present in a reduced amount in the first cell relative to the second cell,
wherein said identified one or more nucleic acid molecules contain a site of modification by the nucleic acid modification enzyme.

36. The method of claim 35, wherein the cleavage moiety is selected from substituted or unsubstituted imidazole (1,3-diazole), triazole, benzimidazole and azaindole; or
wherein the cleavage moiety is selected from the groups represented by formula (I) to (III): 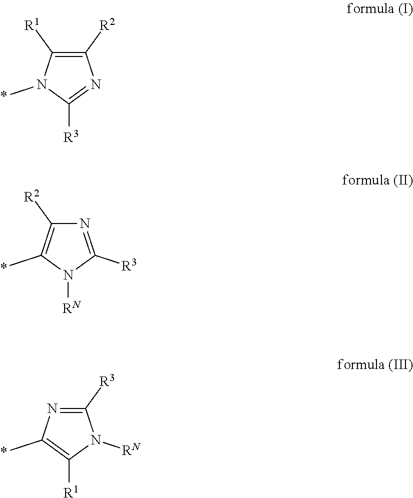
where: R¹, R² and R³ each independently represent a hydrogen atom or a C₁₋₆ alkyl group,
R^{N} represents a hydrogen atom or a C₁₋₆ alkyl group, and
* represents the attachment position with the remainder of the probe (typically the linker unit L),
preferably wherein the cleavage moiety is a group represented by formula (I).

37.-38. (canceled)

39. The method of claim 35, wherein the linker comprises a polyalkylene glycol group.

40. The method of claim 35, wherein the covalent binding moiety is or comprises a group selected from azido (—N₃), nitrone (—R′C═N⁺R″O⁻, where R″ is not H), nitrile oxide (—C≡N⁺—O⁻) or tetrazine.

41. (canceled)

42. The method of claim 35, wherein the partner moiety is or comprises an alkynyl (—C≡C—) group.

43. The method of claim 35, wherein:
(i) the nucleic acid modification enzyme is selected from a nucleic acid methyltransferase, a nucleic acid acetyltransferase, and a nucleic acid glycosyltransferase; and/or
(ii) the cofactor analogue is selected from an S-adenosylmethionine analogue, an acetyl-coenzyme A analogue, and a monosaccharide analogue or a glycan comprising the monosaccharide analogue.

44.-47. (canceled)

48. The method of claim 35, wherein the cofactor analogue is generated in the cell from an exogenous cofactor analogue precursor, preferably wherein the cofactor analogue precursor is selected from a methionine analogue, an acetate analogue and an acetylated monosaccharide analogue.

49.-52. (canceled)

53. A bifunctional probe having the formula:
I-L-B
where I is a substituted or unsubstituted imidazole, L is a linker and B is a binding moiety that covalently binds to a nucleic acid molecule.

54. The bifunctional probe of claim 53 having the formula (4) or formula 5:
I-L-N₃ (4)
where I is a substituted or unsubstituted imidazole, L is a linker and N₃ is an azido group;
I—[PEG]ₙ—N₃ (5)
where I is a substituted or unsubstituted imidazole, PEG is a polyethylene glycol unit, n is 2 to 10 and N₃ is an azido group, preferably wherein n is 4 to 8.

55.-59. (canceled)

60. The bifunctional probe of claim 53 selected from compounds of formula Deg-1 to Deg-3:

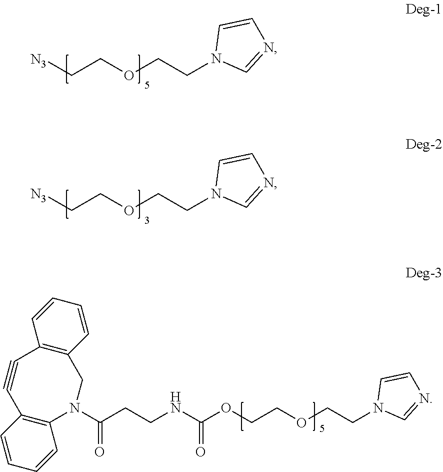

61. (canceled)
